(19) United States
(12) Patent Application Publication
(10) Pub. No.: US 2004/0002818 A1
(43) Pub. Date: Jan. 1, 2004

(54) Method, system and computer software for providing microarray probe data

(75) Inventors: David Kulp, Albany, CA (US); Alan J. Williams, Albany, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, Inc.

(21) Appl. No.: 10/328,818

(22) Filed: Dec. 23, 2002

Related U.S. Application Data
(63) Continuation-in-part of application No. 10/063,559, filed on May 2, 2002.
(63) Continuation-in-part of application No. PCT/US02/13902, filed on May 2, 2002.
(60) Provisional application No. 60/343,511, filed on Dec. 21, 2001.
(60) Provisional application No. 60/349,546, filed on Jan. 18, 2002.
(60) Provisional application No. 60/376,003, filed on Apr. 26, 2002.
(60) Provisional application No. 60/394,574, filed on Jul. 9, 2002.
(60) Provisional application No. 60/403,381, filed on Aug. 14, 2002.

(51) Int. Cl. G06F 19/00; G01N 33/48; G01N 33/50

(52) U.S. Cl. 702/20

(57) ABSTRACT

A genomic web portal system provides correlations between probes of probe arrays, such as probes of microarrays, and biological sequences. The system receives a user selection of probe identifiers, associates the probe identifiers with probe sequences, correlates the probe sequences with the biological sequences; and displays a graphical user interface including graphical elements representing probe sequences associated with the probe identifiers and graphical elements representing the biological sequences. The biological sequences may include sequences of EST, DNA, cDNA, RNA, cRNA, mRNA, gene region, gene, gene transcripts, gene transcription products, chromosome, peptide, protein fragment, protein, and/or residue-counting reference.

### RELATED APPLICATIONS

The present application claims priority from U.S. Provisional Patent Applications Serial Nos. 60/306,033, entitled “PROBESET ANNOTATIONS,” filed Jul. 16, 2001; No. 60/343,511, entitled “METHOD, SYSTEM, AND COMPUTER SOFTWARE FOR PROVIDING A GENOMIC WEB PORTAL,” filed Dec. 21, 2001; No. 60/349,546, entitled “METHOD, SYSTEM, AND COMPUTER SOFTWARE FOR PROVIDING A GENOMIC WEB PORTAL”, filed Jan. 18, 2002; No. 60/376,003, entitled “METHOD, SYSTEM, AND COMPUTER SOFTWARE FOR PROVIDING A GENOMIC WEB PORTAL”, filed Apr. 26, 2002; No. 60/394,574, entitled “METHOD, SYSTEM, AND COMPUTER SOFTWARE FOR PROVIDING A GENOMIC WEB PORTAL”, filed Jul. 9, 2002; No. 60/403,381, entitled “METHOD, SYSTEM, AND COMPUTER SOFTWARE FOR PROVIDING A GENOMIC WEB PORTAL”, filed Aug. 14, 2002, all of which are hereby incorporated herein by reference in their entireties for all purposes. The present application is also a continuation in part of, and claims priority from, U.S. patent application Ser. Nos. 10/063,559, titled “METHOD, SYSTEM, AND COMPUTER SOFTWARE FOR PROVIDING A GENOMIC WEB PORTAL”, filed May 2, 2002; and Patent Cooperation Treaty Patent Application Serial No. PCT/US 02/13902, titled “METHOD, SYSTEM, AND COMPUTER SOFTWARE FOR PROVIDING A GENOMIC WEB PORTAL”, filed May 2, 2002, both of which are hereby incorporated herein by reference in their entireties for all purposes. The present application also is related to U.S. Patent Application, Attorney Docket No. 3291.13, titled “METHOD, SYSTEM AND COMPUTER SOFTWARE FOR PROVIDING GENOMIC ONTOLOGICAL DATA,” filed concurrently herewith and also incorporated by reference herein in its entirety for all purposes.

### BACKGROUND

1. Field of the Invention

The present invention relates to the field of bioinformatics. In particular, the present invention relates to computer systems, methods, and products for providing genomic information over networks such as the Internet.

2. Related Art

Research in molecular biology, biochemistry, and many related health fields increasingly requires organization and analysis of complex data generated by new experimental techniques. These tasks are addressed by the rapidly evolving field of bioinformatics. See, e.g., H. Rashidi and K. Buehler, Bioinformatics Basics: Applications in Biological Science and Medicine (CRC Press, London, 2000); Bioinformatics: A Practical Guide to the Analysis of Gene and Proteins (B. F. Ouelette and A. D. Baxevanis, eds., Wiley & Sons, Inc.; 2d ed., 2001), both of which are hereby incorporated herein by reference in their entireties. Broadly, one area of bioinformatics applies computational techniques to large genomic databases, often distributed over and accessed through networks such as the Internet, for the purpose of illuminating relationships among gene structure and/or location, protein function, and metabolic processes.

### SUMMARY OF THE INVENTION

The expanding use of microarray technology is one of the forces driving the development of bioinformatics. In particular, microarrays and associated instrumentation and computer systems have been developed for rapid and large-scale collection of data about the expression of genes or expressed sequence tags (ESTs) in tissue samples. Data from experiments with genotyping microarrays may be used, among other things, to study genetic characteristics and to detect mutations relevant to genetic and other diseases or conditions. More specifically, the data gained through microarray experiments is valuable to researchers because, among other reasons, many disease states can potentially be characterized by differences in the expression levels of various genes, either through changes in the copy number of the genetic DNA or through changes in levels of transcription (e.g., through control of initiation, provision of RNA precursors, or RNA processing) of particular genes. Thus, for example, researchers use microarrays to answer questions such as: Which genes are expressed in cells of a malignant tumor but not expressed in either healthy tissue or tissue treated according to a particular regime? Which genes or ESTs are expressed in particular organs but not in others? Which genes or ESTs are expressed in particular species but not in others? How does the environment, drugs, or other factors influence gene expression? Data collection is only an initial step, however, in answering these and other questions. Researchers are increasingly challenged to extract biologically meaningful information from the vast amounts of data generated by microarray technologies, and to design follow-on experiments. A need exists to provide researchers with improved tools and information to perform these tasks.

Systems, methods, and computer program products are described herein to address these and other needs. In some embodiments, a web portal processes inquiries regarding biological information, biological devices or substances, reagents, and other information or products related to results of microarray experiments. In some implementations, the user selects “probe-set identifiers” (a broad term that is described below) that may be associated with probe sets of one or more probes. These probe sets are capable of enabling detection of biological molecules. These biological molecules include, but are not limited to, nucleic acids including DNA representations or mRNA transcripts and/or representations of corresponding genes (such nucleic acids may hereafter, for convenience, be referred to simply as “mRNA transcripts”). The corresponding genes or ESTs typically are identified and correlated with related data and/or products, which are provided to the user.

In accordance with a particular embodiment, a system is described for providing correlations between probe-set identifiers and biological sequences. The system includes an input manager that receives a user selection of the probe-set identifiers and a correlator that associates the probe-set identifiers with probe sequences and correlates the probe sequences with the biological sequences. Also included is an output manager that enables for display a graphical user interface including graphical elements representing one or more of the probe sequences associated with the probe-set identifiers and one or more graphical elements representing the biological sequences. The biological sequences may include any one or more of sequences of EST, DNA, cDNA, RNA, cRNA, mRNA, gene region, gene, gene transcripts, gene transcription products, chromosome, peptide, protein fragment, protein, or residue counting reference. In some implementations, the probe-set identifiers or probe sequences are associated with probes of a probe array that detect or measure any one or any combination of biological, biochemical or genetic metrics including gene expression, genotype, SNP, haplotype, or targets including antibodies, cell membrane receptors, monoclonal antibodies and antisera reactive with specific antigenic determinants, drugs, oligonucleotides, nucleic acids, peptides, proteins, cofactors, lectins, sugars, polysaccharides, cells, cellular membranes, or organelles.

In accordance with another embodiment, a method is described for providing correlations between probe-set identifiers and biological sequences. The method includes the acts of associating the probe-set identifiers with probe sequences, and correlating the probe sequences with the biological sequences. Each probe-set identifier may, in some implementations, identify only one probe set comprised of a unique set of one or more probe sequences. Also, in some implementations, the method also includes the act of receiving a user selection of the probe-set identifiers that may be provided, at least in part, using the Internet and/or may be provided, at least in part, using a graphical user interface. In implementations using a graphical user interface, it may include elements for receiving a user selection including a list of probe-set identifiers in a computer-readable file. In these or other implementations it may also include one or more user input elements for receiving a user selection of one or more query biological sequences, or identifiers of one or more query biological sequences. In those implementations, the method further includes the act of associating the one or more query biological sequences, or their identifiers, with one or more probe-set identifiers. The query biological sequences may include, as non-limiting examples, any one or more of sequences of EST, DNA, cDNA, RNA, cRNA, mRNA, gene region, gene, gene transcripts, gene transcription products, chromosome, peptide, protein fragment, or protein. The elements for receiving a user selection further may include a probe-array type interface that accepts a user selection of one or more probe-array types to indicate that the user selection of the probe-set identifiers is to be limited to probe-set identifiers associated with the selected probe-array types.

In these various implementations, the act of associating the query biological sequences, or their identifiers, with the probe-set identifiers may include using a predetermined or a user-selected margin of allowable misalignment between the biological sequence and the probe sequences associated with the probe-set identifiers. Also, the method may further include receiving a selection of the probe-set identifiers that is based, at least in part, on results of at least one experiment with one or more probe arrays having probe sets identified by the probe-set identifiers. In various implementations, the act of correlating may include aligning the probe sequences with the one or more biological sequences, which may be done using a predetermined or a user-selected margin of allowable misalignment. Also in various implementations, the method also includes the act of enabling the correlation for display, which may include providing a graphical user interface having graphical elements representing one or more of the probe sequences associated with the probe-set identifiers, and also having graphical elements representing the biological sequences. The biological sequences may include, as non-limiting examples, any one or more of sequences of EST, DNA, cDNA, RNA, cRNA, mRNA, gene region, gene, gene transcripts, gene transcription products, chromosome, peptide, protein fragment, protein, or residue counting reference. In some implementations, the graphical elements representing the probe sequences are graphically aligned with the graphical elements representing the biological sequences. Optionally, the act of enabling the correlation for display may include providing a graphical user interface having tabular representations of the correlation, which may be in the form of one or more “gene tables,” defined below. The gene tables may include one or more biological sequences, which may have sequences correlated with one or more query sequences. The tabular representations may include one or more probe-set information tables that have, for example, a description of one or more probe sets.

In accordance with another embodiment, an apparatus is described for providing correlations between probe-set identifiers and biological sequences. The apparatus includes an associator that associates the probe-set identifiers with probe sequences, and a correlator that correlates the probe sequences with the biological sequences. In a further embodiment, a system is described for providing correlations between probe-set identifiers and biological sequences. The system includes an input manager that receives a user selection of the probe-set identifiers, and a correlator that associates the probe-set identifiers with probe sequences and correlates the probe sequences with the biological sequences. Yet a further embodiment is described with respect to a system for providing correlations between probe-set identifiers and biological sequences. In this embodiment, the system includes means for receiving a user selection of the probe-set identifiers using a first graphical user interface; means for associating the probe-set identifiers with probe sequences; means for correlating the probe sequences with the biological sequences. The system of this embodiment also includes means for enabling the correlation for display including providing a second graphical user interface including one or more graphical elements representing one or more of the probe sequences associated with the probe-set identifiers and one or more graphical elements representing the one or more biological sequences.

Also described in relation to another embodiment is a computer-implemented graphical user interface for providing correlations between one or more probe-set identifiers and one or more biological sequences. The graphical user interface includes graphical elements representing one or more probe sequences associated with the probe-set identifiers, and graphical elements representing the one or more biological sequences. These graphical elements are graphically aligned with each other. In accordance with yet another embodiment, a graphical user interface is described that is generated by a method for providing correlations between one or more probe-set identifiers and one or more biological sequences. The method includes the acts of associating the probe-set identifiers with probe sequences and correlating the probe sequences with the one or more biological sequences. The graphical user interface includes one or more graphical elements representing the probe sequences graphically aligned with graphical elements representing the biological sequences.

The above implementations are not necessarily inclusive or exclusive of each other and may be combined in any manner that is non-conflicting and otherwise possible, whether they be presented in association with a same, or a different, aspect or implementation. The description of one implementation is not intended to be limiting with respect to other implementations. Also, any one or more function, step, operation, or technique described elsewhere in this specification may, in alternative implementations, be combined with any one or more function, step, operation, or technique described in the summary. Thus, the above implementations are illustrative rather than limiting.

### BRIEF DESCRIPTION OF THE DRAWINGS

The above and further advantages will be more clearly appreciated from the following detailed description when taken in conjunction with the accompanying drawings. In the drawings, like reference numerals indicate like structures or method steps and the leftmost one or two digits of a reference numeral indicate the number of the figure in which the referenced element first appears (for example, the element 180 appears first in FIG. 1; element 1110 appears first in FIG. 11). In functional block diagrams, rectangles generally indicate functional elements, parallelograms generally indicate data, rectangles with curved sides generally indicate stored data, rectangles with a pair of double borders generally indicate predefined functional elements, and keystone shapes generally indicate manual operations. In method flow charts, rectangles generally indicate method steps and diamond shapes generally indicate decision elements. All of these conventions, however, are intended to be typical or illustrative, rather than limiting.

FIG. 1 is a functional block diagram of one embodiment of a probe-array analysis system including an illustrative scanner and an illustrative computer system.

FIG. 2 is a functional block diagram of one embodiment of probe-array analysis applications as illustratively stored for execution in system memory of the computer system of FIG. 1.

FIG. 3 is a functional block diagram of a conventional system for obtaining genomic information over the Internet.

FIG. 4 is a functional block diagram of one embodiment of a genomic portal coupled over the Internet to remote databases and web pages and to clients including networks having user computer systems including that of FIG. 1.

FIG. 5 is a functional block diagram of one embodiment of the genomic portal of FIG. 4 including illustrative embodiments of a database server, portal application computer system, and portal-side Internet server.

FIG. 6 is a simplified graphical representation of one embodiment of computer application platforms for implementing the genomic portal of FIGS. 4 and 5 in communication with clients such as those shown in FIG. 4.

FIG. 7A is a flow chart of one embodiment of a method for providing a user with web pages displaying genomic data and/or genomic product information related, for example, to gene expression, alternative splice variants, differential expression, experimental results, and/or custom probe arrays.

FIG. 7B is a flow chart of one embodiment of a method for receiving and processing a user selection of probe-set identifiers to generate custom design probe arrays and/or custom design probe sets.

FIG. 8 is a functional block diagram of one embodiment of a user-service manager application as may be executed on the portal application computer system of FIG. 5.

FIG. 9 is a simplified graphical representation of one embodiment of a gene or probe-set identifier to products and/or genomics database such as may be by the user-service manager of FIG. 8.

FIG. 10 is a simplified graphical representation of one embodiment of a local genomic and/or product database such as may be accessed by the database server of FIG. 5.

FIG. 11 is a functional block diagram of one embodiment of a gene or EST determiner such as may be included in the user-service manager application of FIG. 8.

FIG. 12 is a functional block diagram of one embodiment of a correlator such as may be included in the user-service manager application of FIG. 8.

FIG. 13A is a graphical representation of one embodiment of a graphical user interface suitable for providing alternative splice variant data to a user based on data correlated by the correlator of FIG. 12.

FIG. 13B is a graphical representation of one embodiment of a graphical user interface suitable for providing alternative splice variant data to a user based on data correlated by the correlator of FIG. 12.

FIG. 14 is a graphical representation of one embodiment of a graphical user interface suitable for providing options and receiving one or more user custom array design and/or custom probe set design selections processed by the gene or EST correlator of FIG. 11.

FIG. 15 is a graphical representation of one embodiment of a graphical user interface suitable for providing one or more custom probe array designs and/or custom probe set designs.

FIG. 16A is a graphical representation of one embodiment of a graphical user interface suitable for providing ontological and related genomic data to a user.

FIG. 16B is a graphical representation of another embodiment of a graphical user interface suitable for providing ontological and related genomic data to a user.

FIG. 17 is a functional block diagram of one embodiment of a genomic data processor as may be included in the user-service manager application of FIG. 8.

FIG. 18A is a graphical representation of one embodiment of a graphical user interface suitable for providing probe-set identifier association data included in local genomic and/or product database of FIG. 10.

FIG. 18B is a graphical representation of one embodiment of database schema suitable for storing graph traversal and connectivity data in the local genomic and/or product database of FIG. 10.

FIG. 19 is a graphical representation of one embodiment of a graphical user interface suitable for providing information pertaining to one or more probe set sequences related to probe-set identifiers of FIG. 2.

FIG. 20 is a graphical representation of one embodiment of a graphical user interface suitable for providing information in a tabular format pertaining to one or more probe sequences of probe sets related to probe-set identifiers of FIG. 2.

### DETAILED DESCRIPTION

The present invention has many preferred embodiments that, in some instances, may include material incorporated from patents, applications and other references for details known to those of the art. When a patent or patent application is referred to below, it should be understood that it is incorporated by reference in its entirety for all purposes. As used in this application, the singular form “a,” “an,” and “the” include plural references unless the context clearly dictates otherwise. For example, the term “an agent” includes a plurality of agents, including mixtures thereof. An individual is not limited to a human being but may also be other organisms including but not limited to mammals, plants, bacteria, or cells derived from any of the above.

Throughout this disclosure, various aspects of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This principle applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques may be had by reference to the examples herein. However, other equivalent conventional procedures may, of course, also be used. Such conventional techniques and descriptions may be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The practice of the present invention may also employ conventional biology methods, software, and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes, and other known devices or media and those that may be developed in the future. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Baxevanis Bioinformatics: A Practical Guide for Analysis of Gene and Proteins (Wiley & Sons, Inc., 2^{nd} ed., 2001).

As will be appreciated by one of skill in the art, the present invention may be embodied as a method, data processing system or program products. Accordingly, the present invention may take the form of data analysis systems, methods, analysis software, and so on. Software written according to the present invention typically is to be stored in some form of computer readable medium, such as memory, or CD-ROM, or transmitted over a network, and executed by a processor. For a description of basic computer systems and computer networks, see, e.g., Introduction to Computing Systems: From Bits and Gates to C and Beyond by Yale N. Patt, Sanjay J. Patel, 1st edition (Jan. 15, 2000) McGraw Hill Text; ISBN: 0072376902; and Introduction to Client/Server Systems: A Practical Guide for Systems Professionals by Paul E. Renaud, 2nd edition (June 1996), John Wiley & Sons; ISBN: 0471133337, both of which are hereby incorporated by reference for all purposes.

Computer software products may be written in any of various suitable programming languages, such as C, C++, Fortran and Java (Sun Microsystems). The computer software product may be an independent application with data input and data display modules. Alternatively, the computer software products may be classes that may be instantiated as distributed objects. The computer software products may also be component software such as Java Beans (Sun Microsystems), Enterprise Java Beans (EJB), Microsoft® COM/DCOM, etc.

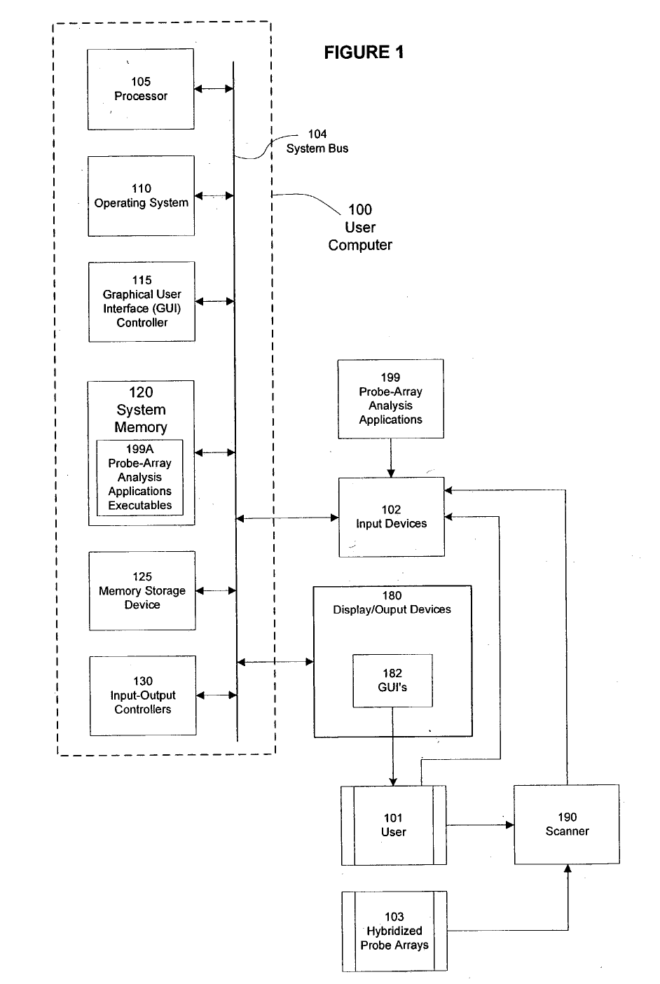

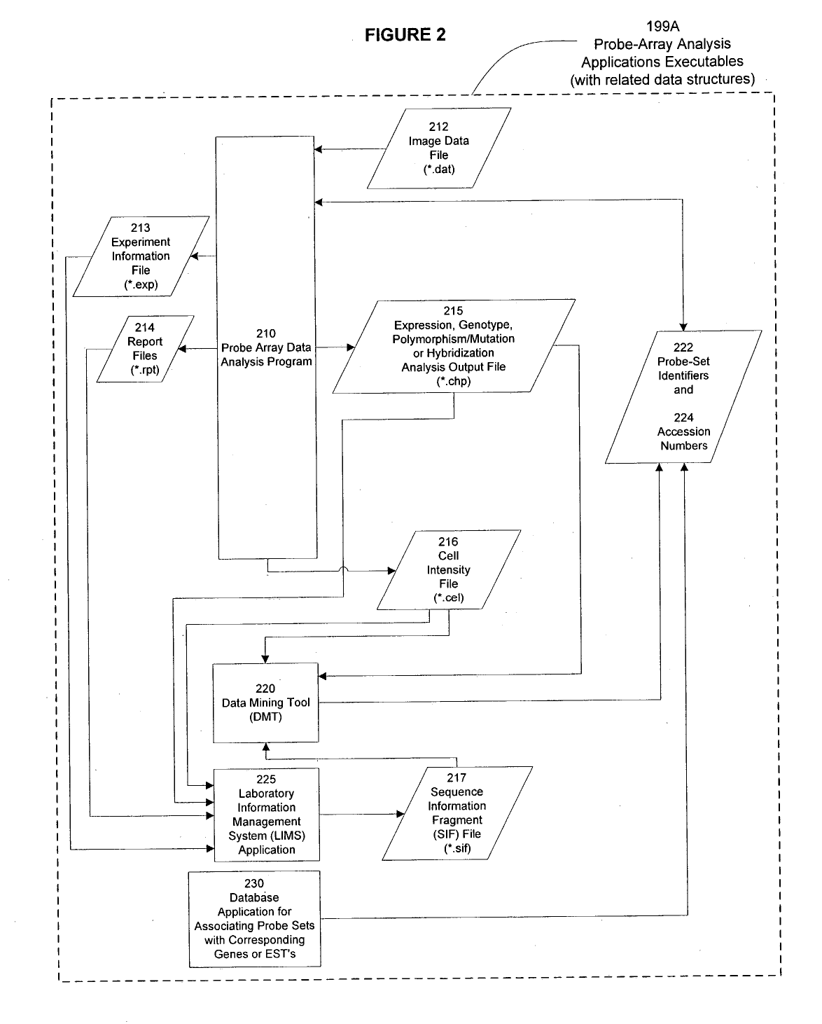

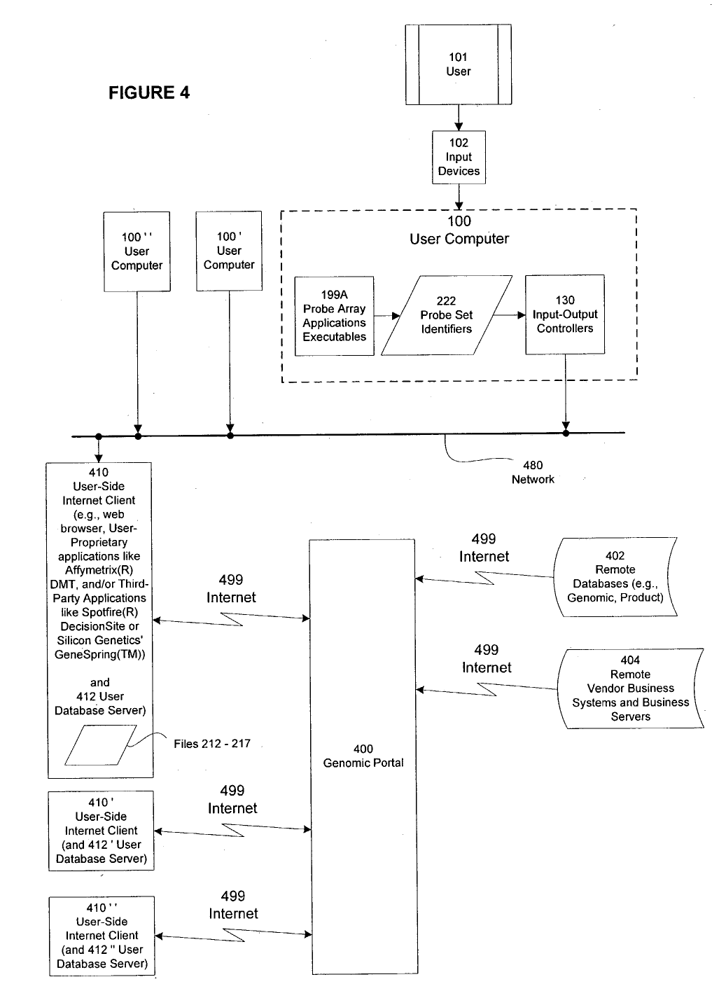

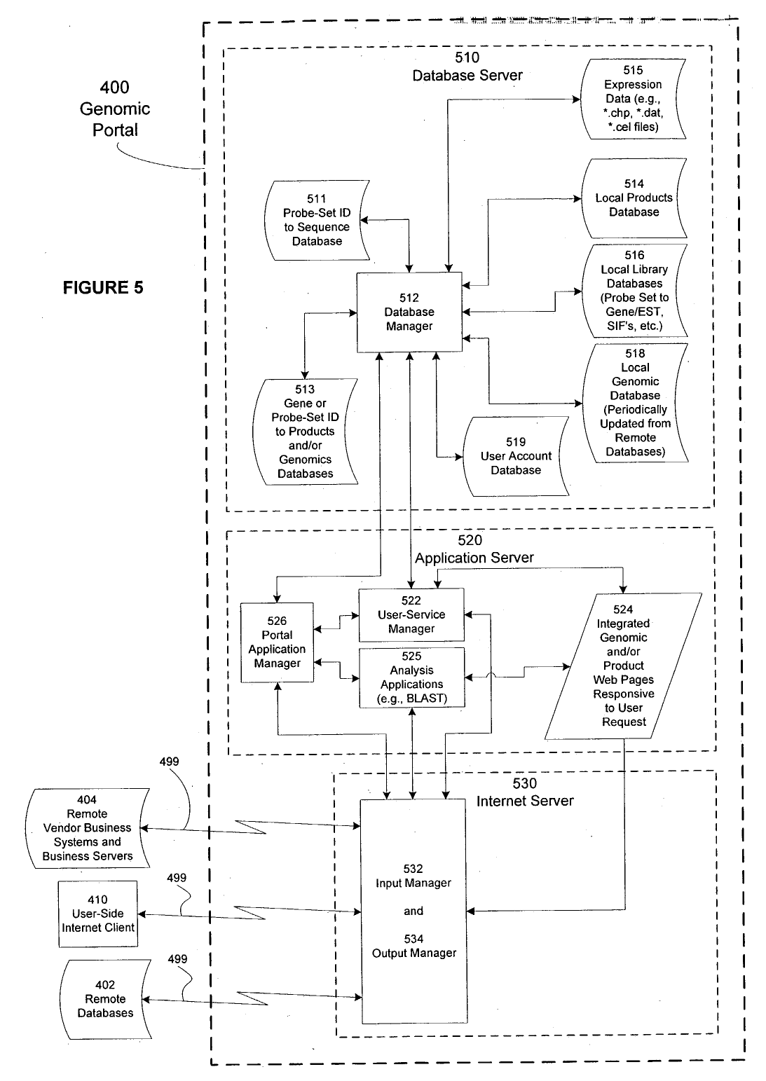

Systems, methods, and computer products are now described with reference to an illustrative embodiment referred to as genomic portal 400. Portal 400 is shown in an Internet environment in FIG. 4, and is illustrated in greater detail in FIGS. 5 through 19. In a typical implementation, portal 400 may be used to provide a user with information related to results from experiments with probe arrays. The experiments often involve the use of scanning equipment to detect hybridization of probe-target pairs, and the analysis of detected hybridization by various software applications, as now described in relation to FIGS. 1 and 2.

Probe Arrays 103: Various techniques and technologies may be used for synthesizing dense arrays of biological materials on or in a substrate or support. For example, Affymetrix GeneChip® arrays are synthesized in accordance with techniques sometimes referred to as VLSIPS™ (Very Large Scale Immobilized Polymer Synthesis) technologies. Some aspects of VLSIPS™ and other microarray and polymer (including protein) array manufacturing methods and techniques have been described in U.S. Pat. No. 09/536,841, International Publication No. WO 00/58516; U.S. Pat. Nos. 5,143,854, 5,242,974, 5,252,743, 5,324,633, 5,445,934, 5,744,305, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846, 6,022,963, 6,083,697, 6,291,183, 6,309,831 and 6,428,752; and in PCT Applications Nos. PCT/US99/00730 (International Publication No. WO 99/36760) and PCT/US01/04285, which are all incorporated herein by reference in their entireties for all purposes.

Patents that describe synthesis techniques in specific embodiments include U.S. Pat. Nos. 6,486,287, 6,147,205, 6,262,216, 6,310,189, 5,889,165, 5,959,098, and 5,412,087, all hereby incorporated by reference in their entireties for all purposes. Nucleic acid arrays are described in many of the above patents, but the same techniques generally may be applied to polypeptide arrays.

Generally speaking, an “array” typically includes a collection of molecules that can be prepared either synthetically or biosynthetically. The molecules in the array may be identical, they may be duplicative, and/or they may be different from each other. The array may assume a variety of formats, e.g., libraries of soluble molecules; libraries of compounds tethered to resin beads, silica chips, or other solid supports; and other formats.

The terms “solid support,” “support,” and “substrate” may in some contexts be used interchangeably and may refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches or wells, or other separation members or elements. In some embodiments, the solid support(s) may take the form of beads, resins, gels, microspheres, or other materials and/or geometric configurations.

Generally speaking, a “probe” typically is a molecule that can be recognized by a particular target. To ensure proper interpretation of the term “probe” as used herein, it is noted that contradictory conventions exist in the relevant literature. The word “probe” is used in some contexts to refer not to the biological material that is synthesized on a substrate or deposited on a slide, as described above, but to what is referred to herein as the “target.”

A target is a molecule that has an affinity for a given probe. Targets may be naturally-occurring or man-made molecules. Also, they can be employed in their unaltered state or as aggregates with other species. The samples or targets are processed so that, typically, they are spatially associated with certain probes in the probe array. For example, one or more tagged targets may be distributed over the probe array.

Targets may be attached, covalently or noncovalently, to a binding member, either directly or via a specific binding substance. Examples of targets that can be employed in accordance with this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term target is used herein, no difference in meaning is intended. Typically, a “probe-target pair” is formed when two macromolecules have combined through molecular recognition to form a complex.

The probes of the arrays in some implementations comprise nucleic acids that are synthesized by methods including the steps of activating regions of a substrate and then contacting the substrate with a selected monomer solution. The term “monomer” generally refers to any member of a set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone. In addition, the terms “biopolymer” and “biological polymer” generally refer to repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer. Related to the term “biopolymer” is the term “biomonomer” that generally refers to a single unit of biopolymer, or a single unit that is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

As used herein, nucleic acids may include any polymer or oligomer of nucleosides or nucleotides (polynucleotides or oligonucleotides) that include pyrimidine and/or purine bases, preferably cytosine, thymine, and uracil, and adenine and guanine, respectively. An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA), which may be isolated from natural sources, recombinantly produced or artificially synthesized and mimetics thereof. A further example of a polynucleotide in accordance with the present invention may be peptide nucleic acid (PNA) in which the constituent bases are joined by peptides bonds rather than phosphodiester linkage, as described in Nielsen et al., Science 254:1497-1500 (1991); Nielsen, Curr. Opin. Biotechnol., 10:71-75 (1999), both of which are hereby incorporated by reference herein. The invention also encompasses situations in which there is a nontraditional base pairing such as Hoogsteen base pairing that has been identified in certain tRNA molecules and postulated to exist in a triple helix. “Polynucleotide” and “oligonucleotide” may be used interchangeably in this application.

Additionally, nucleic acids according to the present invention may include any polymer or oligomer of pyrimidine and purine bases, preferably cytosine (C), thymine (T), and uracil (U), and adenine (A) and guanine (G), respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be deoxyribonucleic acid (DNA) or ribonucleic acid (RNA), or a mixture thereof, and may exist permanently or transitionally in single-stranded or double-stranded form, including homoduplex, heteroduplex, and hybrid states.

As noted, a nucleic acid library or array typically is an intentionally created collection of nucleic acids that can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids that can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired. Nucleic acid arrays that are useful in the present invention include those that are commercially available from Affymetrix, Inc. of Santa Clara, Calif., under the registered trademark “GeneChip®.” Example arrays are shown on the website at affymetrix.com.

In some embodiments, a probe may be surface immobilized. Examples of probes that can be investigated in accordance with this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies. As non-limiting examples, a probe may refer to a nucleic acid, such as an oligonucleotide, capable of binding to a target nucleic acid of complementary sequence through one or more types of chemical bonds, usually through complementary base pairing, usually through hydrogen bond formation. A probe may include natural (i.e. A, G, U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages. Other examples of probes include antibodies used to detect peptides or other molecules, or any ligands for detecting its binding partners. Probes of other biological materials, such as peptides or polysaccharides as non-limiting examples, may also be formed. For more details regarding possible implementations, see U.S. Pat. No. 6,156,501, hereby incorporated by reference herein in its entirety for all purposes. When referring to targets or probes as nucleic acids, it should be understood that these are illustrative embodiments that are not to limit the invention in any way.

Furthermore, to avoid confusion, the term “probe” is used herein to refer to probes such as those synthesized according to the VLSIPS™ technology; the biological materials deposited so as to create spotted arrays; and materials synthesized, deposited, or positioned to form arrays according to other current or future technologies. Thus, microarrays formed in accordance with any of these technologies may be referred to generally and collectively hereafter for convenience as “probe arrays.” Moreover, the term “probe” is not limited to probes immobilized in array format. Rather, the functions and methods described herein may also be employed with respect to other parallel assay devices. For example, these functions and methods may be applied with respect to probe-set identifiers that identify probes immobilized on or in beads, optical fibers, or other substrates or media.

In accordance with some implementations, some targets hybridize with probes and remain at the probe locations, while non-hybridized targets are washed away. These hybridized targets, with their tags or labels, are thus spatially associated with the probes. The term “hybridization” refers to the process in which two single-stranded polynucleotides bind non-covalently to form a stable double-stranded polynucleotide. The term “hybridization” may also refer to triple-stranded hybridization, which is theoretically possible. The resulting (usually) double-stranded polynucleotide is a “hybrid.” The proportion of the population of polynucleotides that forms stable hybrids is referred to herein as the “degree of hybridization.” Hybridization probes usually are nucleic acids (such as oligonucleotides) capable of binding in a base-specific manner to a complementary strand of nucleic acid. Such probes include peptide nucleic acids, as described in Nielsen et al., Science 254:1497-1500 (1991) or Nielsen Curr. Opin. Biotechnol., 10:71-75 (1999) (both of which are hereby incorporated herein by reference), and other nucleic acid analogs and nucleic acid mimetics. The hybridized probe and target may sometimes be referred to as a probe-target pair. Detection of these pairs can serve a variety of purposes, such as to determine whether a target nucleic acid has a nucleotide sequence identical to or different from a specific reference sequence. See, for example, U.S. Pat. No. 5,837,832, referred to and incorporated above. Other uses include gene expression monitoring and evaluation (see, e.g., U.S. Pat. No. 5,800,992 to Fodor, et al.; U.S. Pat. No. 6,040,138 to Lockhart, et al.; and International App. No. PCT/US98/15151, published as WO99/05323, to Balaban, et al.), genotyping (U.S. Pat. No. 5,856,092 to Dale, et al.), or other detection of nucleic acids. The '992, '138, and '092 patents, and publication WO99/05323, are incorporated by reference herein in their entireties for all purposes.

The present invention also contemplates signal detection of hybridization between probes and targets in certain preferred embodiments. See U.S. Pat. Nos. 5,143,854, 5,578,832; 5,631,734; 5,936,324; 5,981,956; 6,025,601 incorporated above and in U.S. Pat. Nos. 5,834,758, 6,141,096; 6,185,030; 6,201,639; 6,218,803; and 6,225,625, in U.S. Patent application No. 60/364,731 and in PCT Application PCT/US99/06097 (published as WO99/47964), each of which also is hereby incorporated by reference in its entirety for all purposes.

A system and method for efficiently synthesizing probe arrays using masks is described in U.S. patent application Ser. No. 09/824,931, filed Apr. 3, 2001, that is hereby incorporated by reference herein in its entirety for all purposes. A system and method for a rapid and flexible microarray manufacturing and online ordering system is described in U.S. Provisional Patent Application Serial No. 60/265,103 filed Jan. 29, 2001, that also is hereby incorporated herein by reference in its entirety for all purposes. Systems and methods for optical photolithography without masks are described in U.S. Pat. No. 6,271,957 and in U.S. patent application Ser. No. 09/683,374 filed Dec. 19, 2001, both of which are hereby incorporated by reference herein in their entireties for all purposes.

As noted, various techniques exist for depositing probes on a substrate or support. For example, “spotted arrays” are commercially fabricated, typically on microscope slides. These arrays consist of liquid spots containing biological material of potentially varying compositions and concentrations. For instance, a spot in the array may include a few strands of short oligonucleotides in a water solution, or it may include a high concentration of long strands of complex proteins. The Affymetrix® 417™ Arrayer and 427™ Arrayer are devices that deposit densely packed arrays of biological materials on microscope slides in accordance with these techniques. Aspects of these and other spot arrayers are described in U.S. Pat. Nos. 6,040,193 and 6,136,269 and in PCT Application No. PCT/US99/00730 (International Publication Number WO 99/36760) incorporated above and in U.S. patent application Ser. No. 09/683,298 hereby incorporated by reference in its entirety for all purposes. Other techniques for generating spotted arrays also exist. For example, U.S. Pat. No. 6,040,193 to Winkler, et al. is directed to processes for dispensing drops to generate spotted arrays. The '193 patent, and U.S. Pat. No. 5,885,837 to Winkler, also describe the use of micro-channels or micro-grooves on a substrate, or on a block placed on a substrate, to synthesize arrays of biological materials. These patents further describe separating reactive regions of a substrate from each other by inert regions and spotting on the reactive regions. The '193 and '837 patents are hereby incorporated by reference in their entireties. Another technique is based on ejecting jets of biological material to form a spotted array. Other implementations of the jetting technique may use devices such as syringes or piezo electric pumps to propel the biological material. It will be understood that the foregoing are non-limiting examples of techniques for synthesizing, depositing, or positioning biological material onto or within a substrate. For example, although a planar array surface is preferred in some implementations of the foregoing, a probe array may be fabricated on a surface of virtually any shape or even a multiplicity of surfaces. Arrays may comprise probes synthesized or deposited on beads, fibers such as fiber optics, glass, silicon, silica or any other appropriate substrate, see U.S. Pat. No. 5,800,992 referred to and incorporated above and U.S. Pat. Nos. 5,770,358, 5,789,162, 5,708,153 and 6,361,947 all of which are hereby incorporated in their entireties for all purposes. Arrays may be packaged in such a manner as to allow for diagnostics or other manipulation in an all inclusive device, see for example, U.S. Pat. Nos. 5,856,174 and 5,922,591 hereby incorporated in their entireties by reference for all purposes.

Probes typically are able to detect the expression of corresponding genes or ESTs by detecting the presence or abundance of cRNA transcripts present in the target. This detection may, in turn, be accomplished in some implementations by detecting labeled cRNA that is derived from cDNA derived from the mRNA in the target.

The terms “mRNA” and “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

In general, a group of probes, sometimes referred to as a probe set, contains sub-sequences in unique regions of the transcripts and does not correspond to a full gene sequence. Further details regarding the design and use of probes and probe sets are provided in PCT Application Serial No. PCT/US 01/02316, filed Jan. 24, 2001 incorporated above; and in U.S. Pat. No. 6,188,783 and in U.S. patent application Ser. No. 09/721,042, filed on Nov. 21, 2000, Ser. No. 09/718,295, filed on Nov. 21, 2000, Ser. No. 09/745,965, filed on Dec. 21, 2000, and Ser. No. 09/764,324, filed on Jan. 16, 2001, all of which patent and patent applications are hereby incorporated herein by reference in their entireties for all purposes.

Scanner 190: FIG. 1 is a functional block diagram of a system that is suitable for, among other things, analyzing probe arrays that have been hybridized with labeled targets. Representative hybridized probe arrays 103 of FIG. 1 may include probe arrays of any type, as noted above. Labeled targets in hybridized probe arrays 103 may be detected using various commercial devices, referred to for convenience hereafter as “scanners.” An illustrative device is shown in FIG. 1 as scanner 190. In some implementations, scanners image the targets by detecting fluorescent or other emissions from the labels, or by detecting transmitted, reflected, or scattered radiation. These processes are generally and collectively referred to hereafter for convenience simply as involving the detection of “emissions.” Various detection schemes are employed depending on the type of emissions and other factors. A typical scheme employs optical and other elements to provide excitation light and to selectively collect the emissions. Also included in some implementations are various light-detector systems employing photodiodes, charge-coupled devices, photomultiplier tubes, or similar devices to register the collected emissions.

Methods and apparatus for signal detection and processing of intensity data are disclosed in, for example, U.S. Pat. Nos. 5,143,854, 5,578,832, 5,631,734, 5,800,992, 5,834,758, 5,856,092, 5,936,324, 5,981,956, 6,025,601, 6,090,555, 6,141,096, 6,185,030, 6,201,639, 6,218,803 and 6,225,625, in U.S. Patent application No. 60/364,731 and in PCT Application PCT/US99/06097 (published as WO99/47964) incorporated above, and in U.S. Pat. Nos. 5,547,839 and 5,902,723 hereby incorporated by reference in their entireties for all purposes. Other scanners or scanning systems are described in U.S. patent application Ser. No. 09/682,837 filed Oct. 23, 2001, Ser. No. 09/683,216 filed Dec. 3, 2001, and Ser. No. 09/683,217 filed Dec. 3, 2001, Ser. No. 09/683,219 filed Dec. 3, 2001,each of which is hereby incorporated by reference in its entirety for all purposes.

The present invention may also make use of various computer program products and software for a variety of purposes, such as probe design, management of data, analysis, and instrument operation. See, U.S. Pat. Nos. 5,593,839, 5,795,716, 5,974,164, 6,090,555, 6,188,783 incorporated above and U.S. Pat. Nos. 5,733,729, 6,066,454, 6,185,561, 6,223,127, 6,229,911 and 6,308,170, hereby incorporated herein in their entireties for all purposes.

Scanner 190 provides data representing the intensities (and possibly other characteristics, such as color) of the detected emissions, as well as the locations on the substrate where the emissions were detected. The data typically are stored in a memory device, such as system memory 120 of user computer 100, in the form of a data file or other data storage form or format. One type of data file, such as image data file 212 shown in FIG. 2, typically includes intensity and location information corresponding to elemental sub-areas of the scanned substrate. The term “elemental” in this context means that the intensities, and/or other characteristics, of the emissions from this area each are represented by a single value. When displayed as an image for viewing or processing, elemental picture elements, or pixels, often represent this information. Thus, for example, a pixel may have a single value representing the intensity of the elemental sub-area of the substrate from which the emissions were scanned. The pixel may also have another value representing another characteristic, such as color. For instance, a scanned elemental sub-area in which high-intensity emissions were detected may be represented by a pixel having high luminance (hereafter, a “bright” pixel), and low-intensity emissions may be represented by a pixel of low luminance (a “dim” pixel). Alternatively, the chromatic value of a pixel may be made to represent the intensity, color, or other characteristic of the detected emissions. Thus, an area of high-intensity emission may be displayed as a red pixel and an area of low-intensity emission as a blue pixel. As another example, detected emissions of one wavelength at a particular sub-area of the substrate may be represented as a red pixel, and emissions of a second wavelength detected at another sub-area may be represented by an adjacent blue pixel. Many other display schemes are known. Two examples of image data are data files in the form *.dat or *.tif as generated respectively by Affymetrix® Microarray Suite based on images scanned from GeneChip® arrays, and by Affymetrix® Jaguar™ software based on images scanned from spotted arrays.

Probe-Array Analysis Applications 199: Generally, a human being may inspect a printed or displayed image constructed from the data in an image file and may identify those cells that are bright or dim, or are otherwise identified by a pixel characteristic (such as color). However, it frequently is desirable to provide this information in an automated, quantifiable, and repeatable way that is compatible with various image processing and/or analysis techniques. For example, the information may be provided for processing by a computer application that associates the locations where hybridized targets were detected with known locations where probes of known identities were synthesized or deposited. Other methods include tagging individual synthesis or support substrates (such as beads) using chemical, biological, electromagnetic transducers or transmitters, and other identifiers. Information such as the nucleotide or monomer sequence of target DNA or RNA may then be deduced. Techniques for making these deductions are described, for example, in U.S. Pat. No. 5,733,729 and in U.S. Pat. No. 5,837,832, noted and incorporated above.

A variety of computer software applications are commercially available for controlling scanners (and other instruments related to the hybridization process, such as hybridization chambers), and for acquiring and processing the image files provided by the scanners. Examples are the Jaguar™ application from Affymetrix, Inc., aspects of which are described in PCT Application PCT/US 01/26390 and in U.S. patent application Ser. Nos. 09/681,819, 09/682,071, 09/682,074, and 09/682,076, and the Microarray Suite application from Affymetrix, filed aspects of which are described in U.S. Provisional Patent Applications, Serial Nos. 60/220,587, 60/220,645, 60/226,999 and 60/312,906, and U.S. patent application Ser. No. 10/219,882, all of which are hereby incorporated herein by reference in their entireties for all purposes. For example, image data in image data file 212 may be operated upon to generate intermediate results such as so-called cell intensity files (*.cel) and chip files (*.chp), generated by Microarray Suite or spot files (*.spt) generated by Jaguar™ software. For convenience, the terms “file” or “data structure” may be used herein to refer to the organization of data, or the data itself generated or used by executables 199A and executable counterparts of other applications. However, it will be understood that any of a variety of alternative techniques known in the relevant art for storing, conveying, and/or manipulating data may be employed, and that the terms “file” and “data structure” therefore are to be interpreted broadly. In the illustrative case in which image data file 212 is derived from a GeneChip® probe array, and in which Microarray Suite generates cell intensity file 216, file 216 may contain, for each probe scanned by scanner 190, a single value representative of the intensities of pixels measured by scanner 190 for that probe. Thus, this value is a measure of the abundance of tagged cRNAs present in the target that hybridized to the corresponding probe. Many such cRNAs may be present in each probe, as a probe on a GeneChip® probe array may include, for example, millions of oligonucleotides designed to detect the cRNAs. The resulting data stored in the chip file may include degrees of hybridization, absolute and/or differential (over two or more experiments) expression, genotype comparisons, detection of polymorphisms and mutations, and other analytical results. In another example, in which executables 199A includes image data from a spotted probe array, the resulting spot file includes the intensities of labeled targets that hybridized to probes in the array. Further details regarding cell files, chip files, and spot files are provided in U.S. Provisional Patent Application Nos. 60/220,645, 60/220,587, and 60/226,999, incorporated by reference above.

In the present example, in which executables 199A may include aspects of Affymetrix® Microarray Suite, the chip file is derived from analysis of the cell file combined in some cases with information derived from library files (not shown) that specify details regarding the sequences and locations of probes and controls. Laboratory or experimental data may also be provided to the software for inclusion in the chip file. For example, an experimenter and/or automated data input devices or programs (not shown) may provide data related to the design or conduct of experiments. As a non-limiting example related to the processing of an Affymetrix® GeneChip® probe array, the experimenter may specify an Affymetrix catalog or custom chip type (e.g., Human Genome U95Av2 chip) either by selecting from a predetermined list presented by Microarray Suite or by scanning a bar code related to a chip to read its type. Microarray Suite may associate the chip type with various scanning parameters stored in data tables including the area of the chip that is to be scanned, the location of chrome borders on the chip used for auto-focusing, the wavelength or intensity of laser light to be used in reading the chip, and so on. Other experimental or laboratory data may include, for example, the name of the experimenter, the dates on which various experiments were conducted, the equipment used, the types of fluorescent dyes used as labels, protocols followed, and numerous other attributes of experiments. As noted, executables 199A may apply some of this data in the generation of intermediate results. For example, information about the dyes may be incorporated into determinations of relative expression. Other data, such as the name of the experimenter, may be processed by executables 199A or may simply be preserved and stored in files or other data structures. Any of these data may be provided, for example over a network, to a laboratory information management server computer, such as user database server 412 of FIG. 4, configured to manage information from large numbers of experiments. Data analysis program 210 may also generate various types of plots, graphs, tables, and other tabular and/or graphical representations of analytical data such as contained in file 215. As will be appreciated by those skilled in the relevant art, the preceding and following descriptions of files generated by executables 199A are exemplary only, and the data described, and other data, may be processed, combined, arranged, and/or presented in many other ways.

The processed image files produced by these applications often are further processed to extract additional data. In particular, data-mining software applications often are used for supplemental identification and analysis of biologically interesting patterns or degrees of hybridization of probe sets. An example of a software application of this type is the Affymetrix® Data Mining Tool, illustrated in FIG. 2 as Data Mining Tool 220 and described in U.S. Provisional Patent Applications, Serial Nos. 60/274,986 and 60/312,256, and U.S. patent application Ser. No. 09/683,980 each of which is hereby incorporated herein by reference in their entireties for all purposes. Software applications also are available for storing and managing the enormous amounts of data that often are generated by probe-array experiments and by the image-processing and data-mining software noted above. An example of these data-management software applications is the Affymetrix® Laboratory Information Management System (LIMS), aspects of which illustrated as Laboratory Information Management System Application 225 and are described in U.S. Provisional Patent Applications, Serial Nos. 60/220,587 and 60/220,645, incorporated above and in U.S. patent application Ser. No. 09/682,098 hereby incorporated by reference herein in its entirety for all purposes. In addition, various proprietary databases accessed by database management software, such as the Affymetrix® EASI (Expression Analysis Sequence Information) database and database software, provide researchers with associations between probe sets and gene or EST identifiers.

For convenience of reference, these types of computer software applications (i.e., for acquiring and processing image files, data mining, data management, and various database and other applications related to probe-array analysis) are generally and collectively represented in FIG. 1 as probe-array analysis applications 199. FIG. 2 is a functional block diagram of probe-array analysis applications 199 as illustratively stored for execution (as executable code 199A corresponding to applications 199) in system memory 120 of user computer 100 of FIG. 1.

As will be appreciated by those skilled in the relevant art, it is not necessary that applications 199 be stored on and/or executed from computer 100; rather, some or all of applications 199 may be stored on and/or executed from an applications server or other computer platform to which computer 100 is connected in a network. For example, it may be particularly advantageous for applications involving the manipulation of large databases, such as Affymetrix® LIMS or Affymetrix® Data Mining Tool (DMT), to be executed from a database server such as user database server 412 of FIG. 4. Alternatively, LIMS, DMT, and/or other applications may be executed from computer 100, but some or all of the databases upon which those applications operate may be stored for common access on server 412 (perhaps together with a database management program, such as the Oracle® 8.0.5 database management system from Oracle Corporation). Such networked arrangements may be implemented in accordance with known techniques using commercially available hardware and software, such as those available for implementing a local-area network or wide-area network. A local network is represented in FIG. 4 by the connection of user computer 100 to user database server 412 (and to user-side Internet client 410, which may be the same computer) via network cable 480. Similarly, scanner 190 (or multiple scanners) may be made available to a network of users over cable 480 both for purposes of controlling scanner 190 and for receiving data input from it.

In some implementations, it may be convenient for user 101 to group probe-set identifiers 222 for batch transfer of information or to otherwise analyze or process groups of probe sets together. For example, as described below, user 101 may wish to obtain annotation information via portal 400 related to one or more probe sets identified by their respective probe-set identifiers. Rather than obtaining this information serially, user 101 may group probe sets together for batch processing. Various known techniques may be employed for associating probe-set identifiers, or data related to those identifiers, together. For instance, user 101 may generate a tab delimited *.txt file including a list of probe-set identifiers for batch processing. This file or another file or data structure for providing a batch of data (hereafter referred to for convenience simply as a “batch file”), may be any kind of list, text, data structure, or other collection of data in any format. The batch file may also specify what kind of information user 101 wishes to obtain with respect to all, or any combination of, the identified probe sets. In some implementations, user 101 may specify a name or other user-specified identifier to represent the group of probe-set identifiers specified in the text file or otherwise specified by user 101. This user-specified identifier may be stored by one of executables 199A, or by elements of portal 400 described below, so that user 101 may employ it in future operations rather than providing the associated probe-set identifiers in a text file or other format. Thus, for example, user 101 may formulate one or more queries associated with a particular user-specified identifier, resulting in a batch transfer of information from portal 400 to user 101 related to the probe-set identifiers that user 101 has associated with the user-specified identifier. Alternatively, user 101 may initiate a batch transfer by providing the text file of probe-set identifiers. In any of these cases, user 101 may formulate queries to obtain, in a single batch operation, probe set records, lists of probe sets sorted into functional groups, protein domain information, sequence homology information, metabolic pathway information, BLAST similarity searches, array content information, and any other information available via portal 400. Similarly, user 101 may provide information, such as laboratory or experimental information, related to a number of probe sets by a batch operation rather than serial ones. The probe sets may be grouped by experiments, by similarity of probe sets (e.g., probe sets representing genes having similar annotations, such as related to transcription regulation), or any other type of grouping. For example, user 101 may assign a user-specified identifier (e.g., “experiments of January 1”) to a series of experiments and submit probe-set identifiers in user-selected categories (e.g., identifying probe sets that were up-regulated by a specified amount) and provide the experimental information to portal 400 for data storage and/or analysis.

User Computer 100: User computer 100, shown in FIG. 1, may be a computing device specially designed and configured to support and execute some or all of the functions of probe array applications 199. Computer 100 also may be any of a variety of types of general-purpose computers such as a personal computer, network server, workstation, or other computer platform now or later developed. Computer 100 typically includes known components such as a processor 105, an operating system 110, a graphical user interface (GUI) controller 115, a system memory 120, memory storage devices 125, and input-output controllers 130. It will be understood by those skilled in the relevant art that there are many possible configurations of the components of computer 100 and that some components that may typically be included in computer 100 are not shown, such as cache memory, a data backup unit, and many other devices. Processor 105 may be a commercially available processor such as a Pentium® processor made by Intel Corporation, a SPARC® processor made by Sun Microsystems, or it may be one of other processors that are or will become available. Processor 105 executes operating system 110, which may be, for example, a Windows®-type operating system (such as Windows NT® 4.0 with SP6a) from the Microsoft Corporation; a Unix® or Linux-type operating system available from many vendors; another or a future operating system; or some combination thereof. Operating system 110 interfaces with firmware and hardware in a well-known manner, and facilitates processor 105 in coordinating and executing the functions of various computer programs that may be written in a variety of programming languages. Operating system 110, typically in cooperation with processor 105, coordinates and executes functions of the other components of computer 100. Operating system 110 also provides scheduling, input-output control, file and data management, memory management, and communication control and related services, all in accordance with known techniques.

System memory 120 may be any of a variety of known or future memory storage devices. Examples include any commonly available random access memory (RAM), magnetic medium such as a resident hard disk or tape, an optical medium such as a read and write compact disc, or other memory storage device. Memory storage device 125 may be any of a variety of known or future devices, including a compact disk drive, a tape drive, a removable hard disk drive, or a diskette drive. Such types of memory storage device 125 typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory 120 and/or the program storage device used in conjunction with memory storage device 125.

In some embodiments, a computer program product is described comprising a computer usable medium having control logic (computer software program, including program code) stored therein. The control logic, when executed by processor 105, causes processor 105 to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers 130 could include any of a variety of known devices for accepting and processing information from a user, whether a human or a machine, whether local or remote. Such devices include, for example, modem cards, network interface cards, sound cards, or other types of controllers for any of a variety of known input devices 102. Output controllers of input-output controllers 130 could include controllers for any of a variety of known display devices 180 for presenting information to a user, whether a human or a machine, whether local or remote. If one of display devices 180 provides visual information, this information typically may be logically and/or physically organized as an array of picture elements, sometimes referred to as pixels. Graphical user interface (GUI) controller 115 may comprise any of a variety of known or future software programs for providing graphical input and output interfaces between computer 100 and user 101, and for processing user inputs. In the illustrated embodiment, the functional elements of computer 100 communicate with each other via system bus 104. Some of these communications may be accomplished in alternative embodiments using network or other types of remote communications.

As will be evident to those skilled in the relevant art, applications 199, if implemented in software, may be loaded into system memory 120 and/or memory storage device 125 through one of input devices 102. All or portions of applications 199 may also reside in a read-only memory or similar device of memory storage device 125, such devices not requiring that applications 199 first be loaded through input devices 102. It will be understood by those skilled in the relevant art that applications 199, or portions of it, may be loaded by processor 105 in a known manner into system memory 120, or cache memory (not shown), or both, as advantageous for execution.

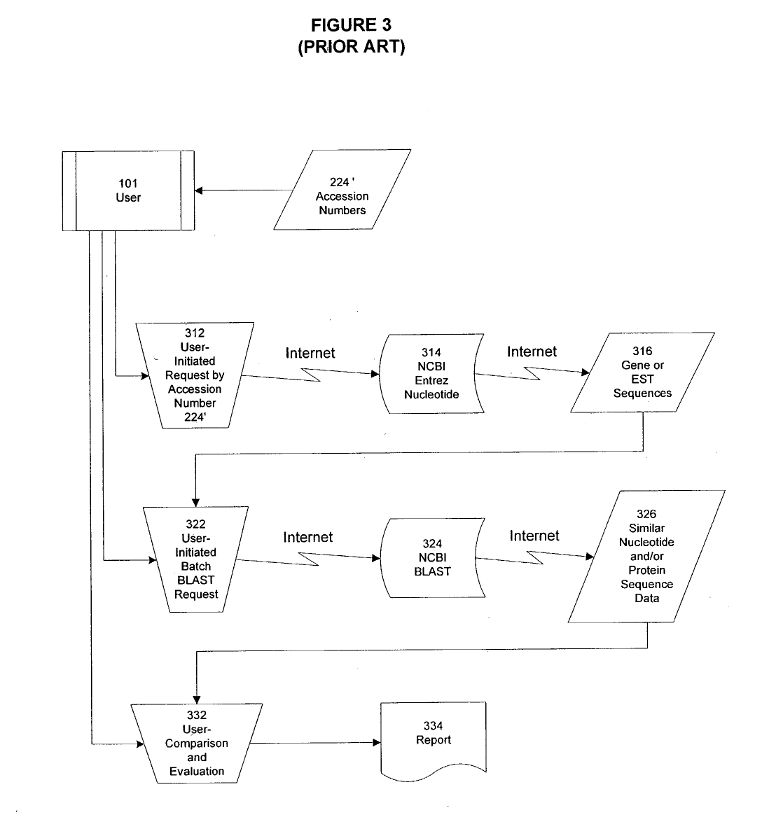

Conventional Techniques for Obtaining Genomic Data: A number of conventional approaches for obtaining genomic data over the Internet are available, some of which are described in the book edited by Ouelette and Baxevanis, incorporated by reference above. FIG. 3 is a functional block diagram representing one simplified example. As shown in FIG. 3, user 101 may consult any of a number of public or other sources to obtain accession numbers 224′. As represented by manual operation 312, user 101 initiates request 312 by accessing through any web browser the Internet web site of the National Center for Biotechnology Information (NCBI) of the National Library of Medicine and the National Institutes of Health (as of November 2002, accessible at the Internet URL http://www.ncbi.nlm.nih.gov/). In particular, user 101 may access the Entrez search and retrieval system that provides information from various databases at NCBI. These databases provide information regarding nucleotide sequences, protein sequences, macromolecular structures, whole genomes, and publication data related thereto. It is illustratively assumed that user 101 accesses in this manner NCBI Entrez nucleotide database 314 and receives information including gene or EST sequences 316. Particularly if accession numbers 224′ represents a large number (e.g., one hundred) of ESTs or genes of interest, as may easily be the case following analysis of probe array experiments, the tasks thus far described may take significant time, perhaps hours.

A genome is all the genetic material in the chromosomes of an organism. In some instances, the term genome may refer to the chromosomal DNA. A genome may be multichromosomal such that the DNA is distributed among a plurality of individual chromosomes in a cell. For example, in human there are 22 pairs of chromosomes plus a gender associated XX or XY pair. DNA derived from the genetic material in the chromosomes of a particular organism is genomic DNA. The term genome may also refer to genetic materials from organisms that do not have chromosomal structure. In addition, the term genome may refer to mitochondria DNA. A genomic library is a collection of DNA fragments that represents the whole or a portion of a genome. Frequently, a genomic library is a collection of clones made from a set of randomly generated, sometimes overlapping DNA fragments representing the entire genome or a portion of the genome of an organism.

User 101 typically copies sequence information from sequences 316 and pastes this information into an HTML document accessible through NCBI's BLAST web pages 324 (as of November 2002, accessible at http://www.ncbi.nlm.nih.gov/BLAST/). This operation, which also may be time consuming and tedious if many sequences are involved, is represented by user-initiated batch BLAST request 322 of FIG. 3. BLAST is an acronym for Basic Local Alignment Search Tool, and, as is well known in the art, consists of similarity search programs that interrogate sequence databases for both protein and DNA using heuristic algorithms to seek local alignments. For example, user 101 may conduct a BLAST search using the “blastn” nucleotide sequence database. Results of this batch BLAST search, represented by similar nucleotide and/or protein sequence data 326, on occasion may not be available to user 101 for many minutes or even hours. User 101 may then initiate comparisons and evaluations 332, which may be conducted manually or using various software tools. User 101 may subsequently issue report 334 interpreting the findings of the searches and positing strategies and requirements for follow-on experiments.

Inputs to Genomic Portal 400 from User 101: The present invention may have preferred embodiments that include methods for providing genetic information over networks such as the Internet as shown in U.S. Patent application No. 60/349,546, incorporated above and U.S. patent application Ser. No. 10/063,559, Nos. 60/376,003, 60/394,574 and 60/403,381, hereby incorporated by reference herein in their entireties for all purposes. FIG. 4 is a functional block diagram showing an illustrative configuration by which user 101 may connect with genomic web portal 400. It will be understood that FIG. 4 is simplified and is illustratively only, and that many implementations and variations of the network and Internet connections shown in FIG. 4 will be evident to those of ordinary skill in the relevant art.

User 101 employs user computer 100 and analysis applications 199 as noted above, including generating and/or accessing some or all of files 212-217. As shown in FIG. 4, files 212-217 are maintained in this example on user database server 412 to which user computer 100 is coupled via network cable 480. Computers 100′, 100″, and computers of other users in a local or wide-area network including an Intranet, the Internet, or any other network may also be coupled to server 412 via cable 480. It will be understood that cable 400 is merely representative of any type of network connectivity, which may involve cables, transmitters, relay stations, network servers, and many other components not shown but evident to those of ordinary skill in the relevant art. Via user computer 100, user 101 may operate a web browser served by user-side Internet client 410 to communicate via Internet 499 with portal 400. Portal 400 may similarly be in communication over Internet 499 with other users and/or networks of users, as indicated by Internet clients 410′ and 410″.

As previously noted, the information provided by user 101 to portal 400 typically includes one or more “probe-set identifiers.” These probe-set identifiers typically come to the attention of user 101 as a result of experiments conducted on probe arrays. For example, user 101 may select probe-set identifiers that identify microarray probe sets capable of enabling detection of the expression of mRNA transcripts from corresponding genes or ESTs of particular interest. As is well known in the relevant art, an EST is a fragment of a gene sequence that may not be fully characterized, whereas a gene sequence generally is complete and fully characterized. The word “gene” is used generally herein to refer both to full size genes of known sequence and to computationally predicted genes. In some implementations, the specific sequences detected by the arrays that represent these genes or ESTs may be referred to as, “sequence information fragments (SIF's)” and may be recorded in a “SIF file,” as noted above with respect to the operations of LIMS 225. In particular implementations, a SIF is a portion of a consensus sequence that has been deemed to best represent the mRNA transcript from a given gene or EST. The consensus sequence may have been derived by comparing and clustering ESTs, and possibly also by comparing the ESTs to genomic sequence information. A SIF is a portion of the consensus sequence for which probes on the array are specifically designed. With respect to the operations of web portal 400, it is assumed with respect to some implementations that some microarray probe sets may be designed to detect the expression of genes based upon sequences of ESTs.

As was described above, the term “probe set” refers in some implementations to one or more probes from an array of probes on a microarray. For example, in an Affymetrix® GeneChip® probe array, in which probes are synthesized on a substrate, a probe set may consist of 30 or 40 probes, half of which typically are controls. These probes collectively, or in various combinations of some or all of them, are deemed to be indicative of a gene, EST, or protein. In a spotted probe array, one or more spots may similarly constitute a “probe set.”

The term “probe-set identifiers” is used broadly herein in that a number of types of such identifiers are possible and are intended to be included within the meaning of this term. One type of probe-set identifier is a name, number, or other symbol that is assigned for the purpose of identifying a probe set. This name, number, or symbol may be arbitrarily assigned to the probe set by, for example, the manufacturer of the probe array. A user may select this type of probe-set identifier by, for example, highlighting or typing the name. Another type of probe-set identifier as intended herein is a graphical representation of a probe set. For example, dots may be displayed on a scatter plot or other diagram wherein each dot represents a probe set. Typically, the dot's placement on the plot represents the intensity of the signal from hybridized, tagged, targets (as described in greater detail below) in one or more experiments. In these cases, a user may select a probe-set identifier by clicking on, drawing a loop around, or otherwise selecting one or more of the dots. In another example, user 101 may select a probe-set identifier by selecting a row or column in a table or spreadsheet that correlates probe sets with accession numbers and other genomic information.

Yet another type of probe-set identifier, as that term is used herein, includes a nucleotide or amino acid sequence. For example, it is illustratively assumed that a particular SIF is a unique sequence of 500 bases that is a portion of a consensus sequence or exemplar sequence gleaned from EST and/or genomic sequence information. It further is assumed that one or more probe sets are designed to represent the SIF. A user who specifies all or part of the 500-base sequence thus may be considered to have specified all or some of the corresponding probe sets.

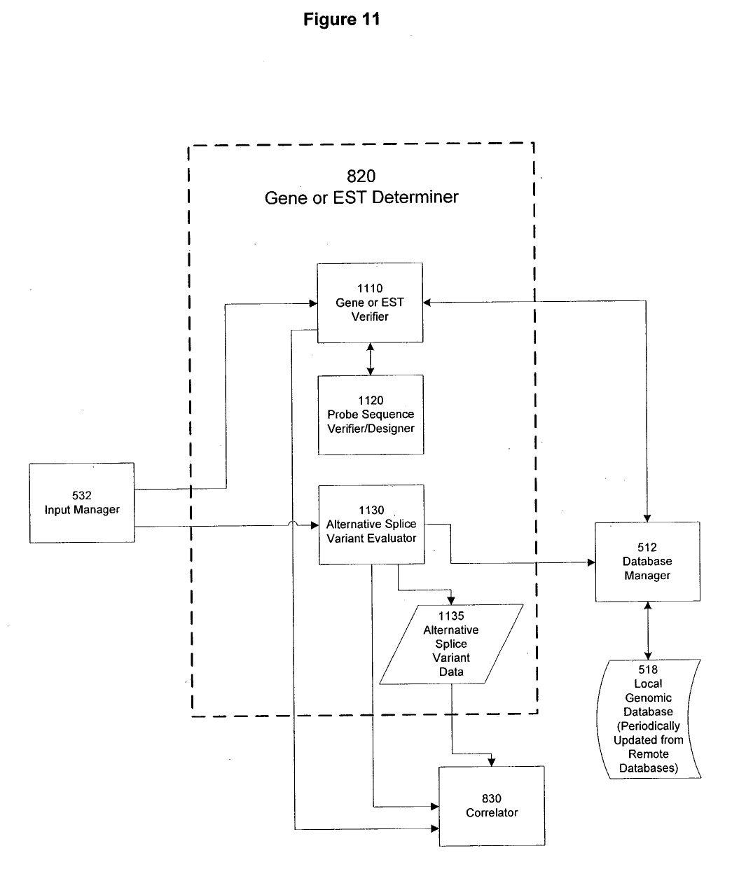

In yet another example, a user may specify a SIF, gene, protein, or EST sequence for which there are no corresponding probe sets. The user requests to have a corresponding probe set produced for the specified sequence. User-service manager 522 (described below) assigns an identifier for the new probe set and this identifier, together with the sequence or sequences from which the probes are to be designed, are stored by database manager 512 in one or more databases. Manager 522 may submit and/or design probe sets for the corresponding SIF, gene, or EST and correlates the probe sets with the new probe-set identifiers. Further details regarding the processing and implementation of custom probe designs are provided in U.S. Provisional Patent Applications Nos. 60/301,298, and 60/265,103; and U.S. patent applications Ser. Nos. 09/824,931, and 10/065,868; each of which is hereby incorporated by reference herein in its entirety for all purposes. Additional aspects of probe design are described below in relation to the operations of probe sequence verifier/designer 1120 and other elements of the illustrative implementation of FIG. 11.

As a further example with respect to a particular implementation, a user may specify a portion of the 500-base sequence noted above, which may be unique to that SIF, or, alternatively, may also identify another SIF, EST, cluster of ESTs, consensus sequence, and/or gene or protein. The user thus specifies a probe-set identifier for one or more genes or ESTs. In another variation, it is illustratively assumed that a particular SIF is a portion of a particular consensus sequence. It is further assumed that a user specifies a portion of the consensus sequence that is not included in the SIF but that is unique to the consensus sequence or the gene or ESTs the consensus sequence is intended to represent. In that case, the sequence specified by the user is a probe-set identifier that identifies the probe set corresponding to the SIF, even though the user-specified sequence is not included in the SIF. Parallel cases are possible with respect to user specifications of partial sequences of ESTs and genes or ESTs, as those skilled in the relevant art will now appreciate.

A further example of a probe-set identifier is an accession number of a gene or EST. Gene and EST accession numbers are publicly available. A probe set may therefore be identified by the accession number or numbers of one or more ESTs and/or genes corresponding to the probe set. The correspondence between a probe set and ESTs or genes may be maintained in a suitable database, such as that accessed by database application 230 or local library databases 516, from which the correspondence may be provided to the user. Similarly, gene fragments or sequences other than ESTs may be mapped (e.g., by reference to a suitable database) to corresponding genes or ESTs for the purpose of using their publicly available accession numbers as probe-set identifiers. For example, a user may be interested in product or genomic information related to a particular SIF that is derived from EST-1 and EST-2. The user may be provided with the correspondence between that SIF (or part or all of the sequence of the SIF) and EST-1 or EST-2, or both. To obtain product or genomic data related to the SIF, or a partial sequence of it, the user may select the accession numbers of EST-1, EST-2, or both.

Additional examples of probe-set identifiers include one or more terms that may be associated with the annotation of one or more gene or EST sequences, where the gene or EST sequences may be associated with one or more probe sets. For convenience, such terms may hereafter be referred to as “annotation terms” and will be understood to potentially include, in various implementations, one or more words, graphical elements, characters, or other representational forms that provide information that typically is biologically relevant to or related to the gene or EST sequence. Associations between the probe-set identifier terms and gene or EST sequences may be stored in a database such as Probe-set ID to sequence database 511, local genomic database 518, or they may be transferred from remote databases 402. Examples of such terms associated with annotations include those of molecular function (e.g. transcription initiation), cellular location (e.g. nuclear membrane), biological process (e.g. immune response), tissue type (e.g. kidney), or other annotation terms known to those in the relevant art.

To provide a further specific example, user 101 may input the illustrative annotation term “tumor suppression.” A large number of genes or ESTs are known to be involved with this biological process. For example, a gene known as p53 is involved with tumor suppression, and this information is stored in one or more of the databases accessible from database server 410. Portal 400 provides to user 101 a list of probe-set identifiers that includes the one or more probe-set identifiers associated with gene p53. The list of probe-set identifiers may be provided to the user in one of numerous possible formats. For example, the format may include a table comprising all the probe sets associated with all the genes or ESTs associated with “tumor suppression.” Alternatively, the format may separate the probe sets related to each gene or EST into its own table.

Genomic web portal 400: Genomic web portal 400 provides to user 101 data related to one or more genes, ESTs, or proteins. Feature elements that make up a gene include: exons, 5′ and 3′ untranslated regions, coding regions, start and stop codons, introns, 5′ transcriptional control elements, 3′ polyadenylation signals, splice site boundaries, and protein-based annotations of the coding regions. In the present implementation, an EST or protein may include what those of ordinary skill in the related art refer to as alternative splice variants. An alternative splice variant generally refers to ESTs or proteins that are derived from a specific composition and arrangement of exons, or coding regions, from a genomic DNA sequence or gene. A molecular apparatus commonly referred to as the “splicesome” performs a process referred to as RNA processing after a gene has been transcribed into a primary RNA transcript. The splicesome cleaves the primary RNA transcript at specific locations that include the intron/exon boundaries. After cleavage, the splicesome rearranges the cleaved sequence and splices the sequence together, generally leaving out the intron sequences and possibly leaving out one or more exon sequences. The splicesome may produce alternative splice variants by altering the number, arrangement, and/or content (i.e., by splicing one or more intron/exon portions) of exons. Thus, alternative splice variants could also include the arrangement of partial sequence from exons that, for instance, may include alternative 3′ and 5′ splice sites. Those of ordinary skill in the related art will appreciate that approximately a third to over half of all human genes produce multiple transcript variants (E. S. Lander, et al., “Initial sequencing and analysis of the human genome,” Nature, vol. 409, pp. 860-921., 2001; A. A. Mironov, J. W. Fickett, and M. S. Gelfand, “Frequent alternative splicing of human genes,” Genome Res, vol. 9, pp. 1288-93., 1999), hereby incorporated by reference herein in their entireties. Each alternative splice variant could have different expression patterns and function. It is also generally appreciated that alternative splicing is an important regulatory mechanism in higher eukaryotes. For example, a gene could include three exons that for the purposes of illustration may be referred to as exon 1, exon 2, and exon 3. In the present example, a plurality of alternative splice variants from that gene are possible that could include an EST composed of exons 1, 2, and 3; another EST composed of exons 1, and 2; or an EST composed of exons 1 and 3.

Typically, each gene or EST has at least one corresponding probe set that is identified by a probe-set identifier that, as just noted, may be a number, name, accession number, symbol, graphical representation (e.g., dot or highlighted tabular entry), and/or nucleotide sequence, as illustrative and non-limiting examples. The corresponding probe sets are capable of enabling detection of the expression of their corresponding gene or alternative splice variant. In some embodiments a probe set designed to recognize the mRNA expression of a gene may identify one or more alternative splice variants. In some cases a plurality of probe sets may be capable of identifying a specific alternative splice variant.

In a preferred embodiment, probe sets are designed to identify specific alternative splice variants. For example, a probe set may consist of probes designed to interrogate the exons of a particular alternative splice variant as well as junction probes designed to interrogate the region where two specific exons are predicted to be joined together. The junction probe may interrogate, for instance, the sequence of the 3′ end of exon 1 and the 5′ end of exon 3. In the present example, an alternative splice variant mRNA that comprises exons 1 and 3 will hybridize to the exon probes and, if the splice variant is joined in the correct orientation, it will also hybridize to the one or more junction probes. Additional examples of alternative splice variant probe sets and probe arrays are described in U.S. patent application Ser. No. 09/697,877; and U.S. Provisional Patent Applications Nos. 60/362,315, 60/362,524, each of which is hereby incorporated by reference herein in its entirety for all purposes.

In response to a user selection of one or more probe-set identifiers, portal 400 provides user 101 with one or more of genomic, EST, protein, or annotation information and/or information regarding biological products. This information may be helpful to user 101 in analyzing the results of experiments and in designing or implementing follow-up experiments.

FIG. 5 is a functional block diagram of one of many possible embodiments of portal 400. In this example, portal 400 has hardware components including three computer platforms: database server 510, Internet server 530, and application server 520. Various functional elements of portal 400, such as database manager 512, input and output managers 532 and 534, and user-service manager 522, carry out their operations on these computer platforms. That is, in a typical implementation, the functions of managers 512, 532, 534, and 522 are carried out by the execution of software applications on and across the computer platforms represented by servers 510, 530, and 520. Portal 400 is described first with respect to its computer platforms, and then with respect to its functional elements.

Each of servers 510, 520 and 530 may be any type of known computer platform or a type to be developed in the future, although they typically will be of a class of computer commonly referred to as servers. However, they may also be a main frame computer, a work station, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen. Appropriate operating systems include Windows NT®, Sun Solaris, Linux, OS/400, Compaq Tru64 Unix, SGI IRIX, Siemens Reliant Unix, and others.

There may be significant advantages to carrying out the functions of portal 400 on multiple computer platforms in this manner, such as lower costs of deployment, database switching, or changes to enterprise applications, and/or more effective firewalls. Other configurations, however, are possible. For example, as is well known to those of ordinary skill in the relevant art, so-called two-tier or N-tier architectures are possible rather than the three-tier server-side component architecture represented by FIG. 5. See, for example, E. Roman, Mastering Enterprise JavaBeans™ and the Java™2 Platform (Wiley & Sons, Inc., NY, 1999) and J. Schneider and R. Arora, Using Enterprise Java™ (Que Corporation, Indianapolis, 1997), both of which are hereby incorporated by reference in their entireties for all purposes.

It will be understood that many hardware and associated software or firmware components that may be implemented in a server-side architecture for Internet commerce are not shown in FIG. 5. Components to implement one or more firewalls to protect data and applications, uninterruptible power supplies, LAN switches, web-server routing software, and many other components are not shown. Similarly, a variety of computer components customarily included in server-class computing platforms, as well as other types of computers, will be understood to be included but are not shown. These components include, for example, processors, memory units, input/output devices, buses, and other components noted above with respect to user computer 100. Those of ordinary skill in the art will readily appreciate how these and other conventional components may be implemented.

The functional elements of portal 400 also may be implemented in accordance with a variety of software facilitators and platforms (although it is not precluded that some or all of the functions of portal 400 may also be implemented in hardware or firmware). Among the various commercial products available for implementing e-commerce web portals are BEA WebLogic from BEA Systems, which is a so-called “middleware” application. This and other middleware applications are sometimes referred to as “application servers,” but are not to be confused with application server 520, which is a computer. The function of these middleware applications generally is to assist other software components (such as managers 512, 522, or 532) to share resources and coordinate activities. The goals include making it easier to write, maintain, and change the software components; to avoid data bottlenecks; and prevent or recover from system failures. Thus, these middleware applications may provide load-balancing, fail-over, and fault tolerance, all of which features will be appreciated by those of ordinary skill in the relevant art.

Other development products, such as the Java™ 2 platform from Sun Microsystems, Inc. may be employed in portal 400 to provide suites of applications programming interfaces (API's) that, among other things, enhance the implementation of scalable and secure components. The platform known as J2EE (Java™2, Enterprise Edition), is configured for use with Enterprise JavaBeans™, both from Sun Microsystems. Enterprise JavaBeans™ generally facilitates the construction of server-side components using distributed object applications written in the Java™ language. Thus, in one implementation, the functional elements of portal 400 may be written in Java and implemented using J2EE and Enterprise JavaBeans™. Various other software development approaches or architectures may be used to implement the functional elements of portal 400 and their interconnection, as will be appreciated by those of ordinary skill in the art.

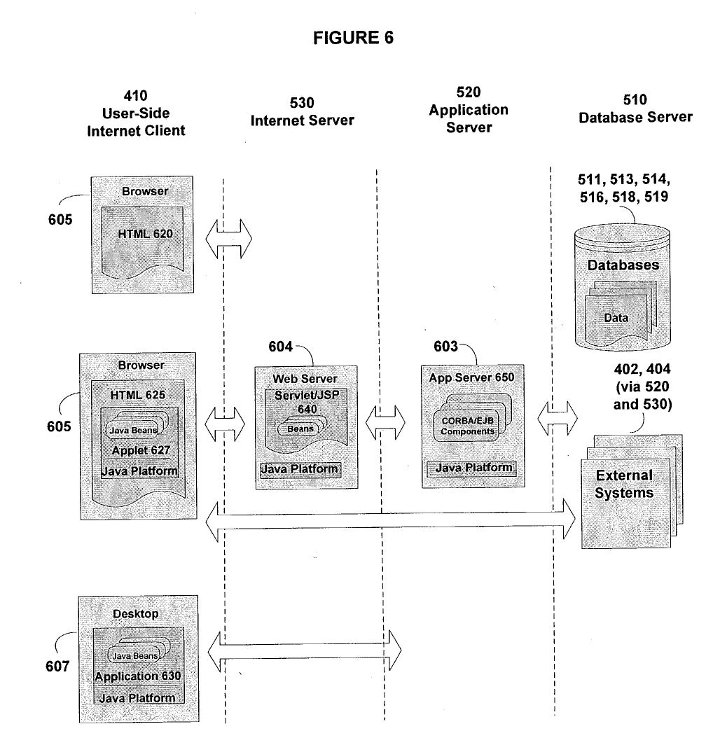

One implementation of these platforms and components is shown in FIG. 6. FIG. 6 is a simplified graphical representation of illustrative interactions between user-side internet client 410 on the user side and input and output managers 532 and 534 of Internet server 530 on the portal side, as well as communications among the three tiers (servers 510, 520, and 530) of portal 400. Browser 605 on client 410 sends and receives HTML documents 620 to and from server 530. HTML document 625 includes applet 627. Browser 605, running on user computer 100, provides a run-time container for applet 627. Functions of managers 532 and 534 on server 530, such as the performance of GUI operations, may be implemented by servlet and/or JSP 640 operating with a Java™ platform. A servlet engine executing on server 530 provides a runtime container for servlet 640. JSP (Java Server Pages) from Sun Microsystems, Inc. is a script-like environment for GUI operations; an alternative is ASP (Active Server Pages) from the Microsoft Corporation. App server 650 is the middleware product referred to above, and executes on application server 520. EJB (Enterprise JavaBeans™ is a standard that defines an architecture for enterprise beans, which are application components. CORBA (Common Object Request Broker Architecture) similarly is a standard for distributed object systems, i.e., the CORBA standards are implemented by CORBA-compliant products such as Java™ IDL. An example of an EJB-compliant product is WebLogic, referred to above. Further details of the implementation of standards, platforms, components, and other elements for an Internet portal and its communications with clients, are well known to those skilled in the relevant art.

As noted, one of the functional elements of portal 400 is input manager 532. Manager 532 receives a set, i.e., one or more, of probe-set identifiers from user 101 over Internet 499. Manager 532 processes and forwards this information to user-service manager 522. These functions are performed in accordance with known techniques common to the operation of Internet servers, also commonly referred to in similar contexts as presentation servers. Another of the functional elements of portal 400 is output manager 534. Manager 534 provides information assembled by user-service manager 522 to user 101 over Internet 499, also in accordance with those known techniques, aspects of which were described above in relation to FIG. 6. The information assembled by manager 522 is represented in FIG. 5 as data 524, labeled “integrated genomic and/or product web pages responsive to user request.” The data is integrated in the sense, among other things, that it is based, at least in part, on the specification by user 101 of probe-set identifiers and thus has common relationships to the genes and/or ESTs, or proteins corresponding to those identifiers. The presentation by manager 534 of data 524 may be implemented in accordance with a variety of known techniques. As some examples, data 524 may include HTML or XML documents, email or other files, or data in other forms. The data may include Internet URL addresses so that user 101 may retrieve additional HTML, XML, or other documents or data from remote sources.

Portal 400 further includes database manager 512. In the illustrated embodiment, database manager 512 coordinates the storage, maintenance, supplementation, and all other transactions from or to any of local databases 511, 513, 514, 516, and 518. Manager 512 may undertake these functions in cooperation with appropriate database applications such as the Oracle® 8.0.5 database management system.

In some implementations, manager 512 periodically updates local genomic database 518. The data updated in database 518 includes data related to genes, ESTs, or proteins that correspond with one or more probe sets. The probe sets may be those used or designed for use on any microarray product, and/or that are expected or calculated to be used in microarray products of any manufacturer or researcher. For example, the probe sets may include all probe sets synthesized on the line of stocked GeneChip® probe arrays from Affymetrix, Inc., including its Arabidopsis Genome Array, CYP450 Array, Drosophila Genome Array, E. coli Genome Array, GenFlex™ Tag Array, HIV PRT Plus Array, HuGeneFL Array, Human Genome U95 Set, Human Genome U133 Set, HuSNP Probe Array, Murine Genome U74 Set, P53 Probe Array, Rat Genome U34 Set, Rat Neurobiology U34 Set, Rat Toxicology U34 Array, Human Genome Focus Array, or Yeast Genome S98 Array. The probe sets may also include those synthesized on alternative splice arrays or custom arrays for user 101 or others. However, the data updated in database 518 need not be so limited. Rather, it may relate, e.g., to any number of genes, ESTs, or proteins. Types of data that may be stored in database 518 are described below in relation to the operations of manager 522 in directing the periodic collection of this data from remote sources providing the locally maintained data in database 518 to users.

Database 516 includes data of a type referred to above in relation to database application 230, i.e., data that associates probe sets with their corresponding gene or EST and their identifiers. Database 516 may also include SIF's, and other library data. User-service manager 522 may provide database manager 512 from time to time with update information regarding library and other data. In some cases, this update information will be provided by the owners or managers of proprietary information, although this information may also be made available publicly, as on a web site, for uploading.

Information for storage by manager 512 in local products database 514 may similarly be provided by vendors, distributors, or agents, or obtained from public sources such as web sites. A wide variety of product-related information may be included in database 514, examples of which include availability, pricing, composition, suitability, or ordering data. The information may relate to a wide variety of products, including any type of biological device or substance, or any type of reagent that may be used with a biological device or substance. To provide just a few examples, the device, substance, or reagent may be an oligonucleotide, probe array, clone, antibody, or protein. The data stored in database 514 may also include links, such as Internet URL addresses, to remote sites where product data is available, such as vendors' web sites.

Database 511 includes information relating probe-set identifiers to the sequences of the probes. This information may be provided by the manufacturer of the probes, the researchers who devise probes for spotted arrays or other custom arrays, or others. Moreover, the application of portal 400 is not limited to probes arranged in arrays. As noted, probes may be immobilized on or in beads, optical fibers, or other substrates or media. Thus, database 511 may also include information regarding the sequences of these probes.

Database 519 includes information about users and their accounts for doing business with or through portal 400. Any of a variety of account information, such as current orders, past orders, and so on, may be obtained from users, all as will be readily apparent to those of ordinary skill in the art. Also, information related to users may be developed by recording and/or analyzing the interactions of users with portal 400, in accordance with known techniques used in e-commerce. For example, user-service manager 522 may take note of users' areas of genomic interest, their purchase or product-inquiry activities, the frequency of their accessing of various services, and so on, and provide this information to database manager 512 for storage or update in database 519.

Another functional element of portal 400 is user-service manager 522. Among other functions, manager 522 may periodically cause database manager 512 to update local genomic database 518 from various sources, such as remote databases 402. For example, according to any chronological schedule (e.g., daily, weekly, etc.), or need-driven schedule (e.g., in response to a user making an authorized request for updated information), manager 522 may, in accordance with known techniques, initiate searches of remote databases 402 by formulating appropriate queries, addressed to the URL's of the various databases 402, or by other conventional techniques for conducting data searches and/or retrieving data or documents over the Internet. These search queries and corresponding addresses may be provided in a known manner to output manager 534 for presentation to databases 402. Input manager 532 receives replies to the queries and provides them to manager 522, which then provides them to database manager 512 for updating of database 518, all in accordance with any of a variety of known techniques for managing information flow to, from, and within an Internet site.

Portal application manager 526 manages the administrative aspects of portal 400, possibly with the assistance of a middleware product such as an applications server product. One of these administrative tasks may be the issuance of periodic instructions to manager 522 to initiate the periodic updating of database 518 just described. Alternatively, manager 522 may self-initiate this task. It is not required that all data in database 518 be updated according to the same periodic schedule. Rather, it may be typical for different types of data and/or data from different sources to be updated according to different schedules. Moreover, these schedules may be changed, and need not be according to a consistent schedule. That is, updating for particular data may occur after a day, then again after 2 days, then at a different period that may continue to vary. Numerous factors may influence the determination by manager 526 or manager 522 to maintain or vary these periods, such as the response time from various remote databases 402, the value and/or timeliness of the information in those databases, cost considerations related to accessing or licensing the databases, the quantity of information that must be accessed, and so on.

In some implementations, manager 522 constructs from data in local genomic database 518 a set of data related to genes, ESTs, or proteins corresponding to the set of probe-set identifiers selected by user 101. The user selection may be forwarded to manager 522 by input manager 532 in accordance with known techniques. Manager 522, also in accordance with known techniques, obtains the data from database 518 by forming appropriate queries, such as in one of the varieties of SQL language, based on the user selection. Manager 522 then forwards the queries to database manager 512 for execution against database 518. Other techniques for extracting information from database 518 may be used in alternative implementations.

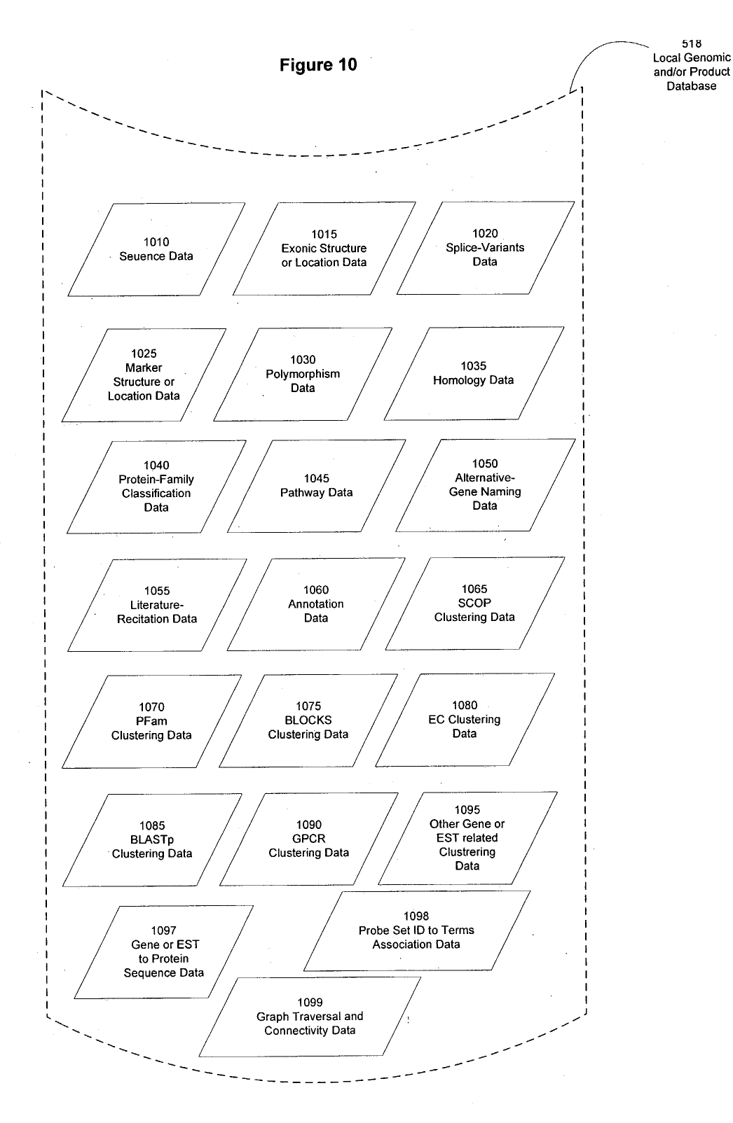

As noted, various types of data may be accessed from remote databases 402 and maintained in local genomic database 518. Examples are illustrated in FIG. 10 that include sequence data 1010, exonic structure or location data 1015, splice-variants data 1020, marker structure or location data 1025, polymorphism data 1030, homology data 1035, protein-family classification data 1040, pathway data 1045, alternative-gene naming data 1050, literature-recitation data 1055, and annotation data 1060. Many other examples are possible. Also, genomic data not currently available but that becomes available in the future may be accessed and locally maintained as described herein. Examples of remote databases 402 currently suitable for accessing in the manner described include GenBank, GenBank New, SwissProt, GenPept, DB EST, Unigene, PIR, Prosite, Pfam, Prodom, Blocks, PDB, PDBfinder, EC Enzyme, Kegg Pathway, Kegg Ligand, OMIM, OMIM Map, OMIM Allele, DB SNP, Gene Ontology, SeqStore®, PubMed, SWALL, InterPro, and LocusLink. Hundreds of other databases currently exist that are suitable, any many more will be developed in the future that may be included as aspects of databases 402, and thus this list is merely illustrative.

Moreover, local genomic database 518 may also be supplemented with data obtained or deduced (by user-service manager 522) from other of the local databases serviced by database manager 512. In particular, although local products database 514 is shown for convenience of illustration as separate from database 518, it may be the same database. Alternatively, or all or part of the data in database 514 may be duplicated in, or accessible from, database 518. Also, in some implementations, data may be retrieved from one or more of remote databases 402 in real time with respect to a user request rather than from locally maintained database 518.

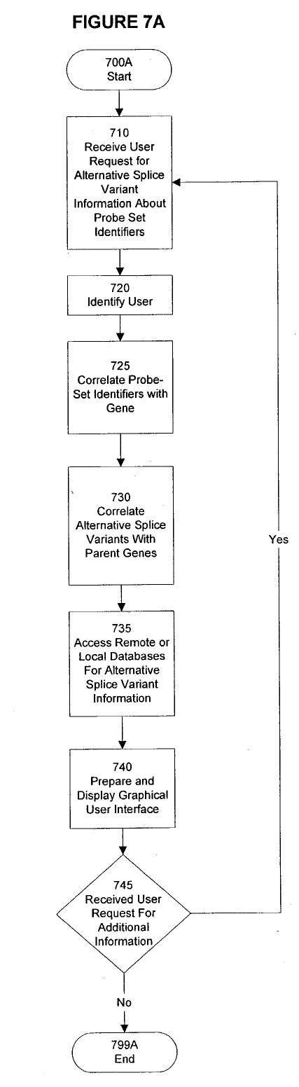

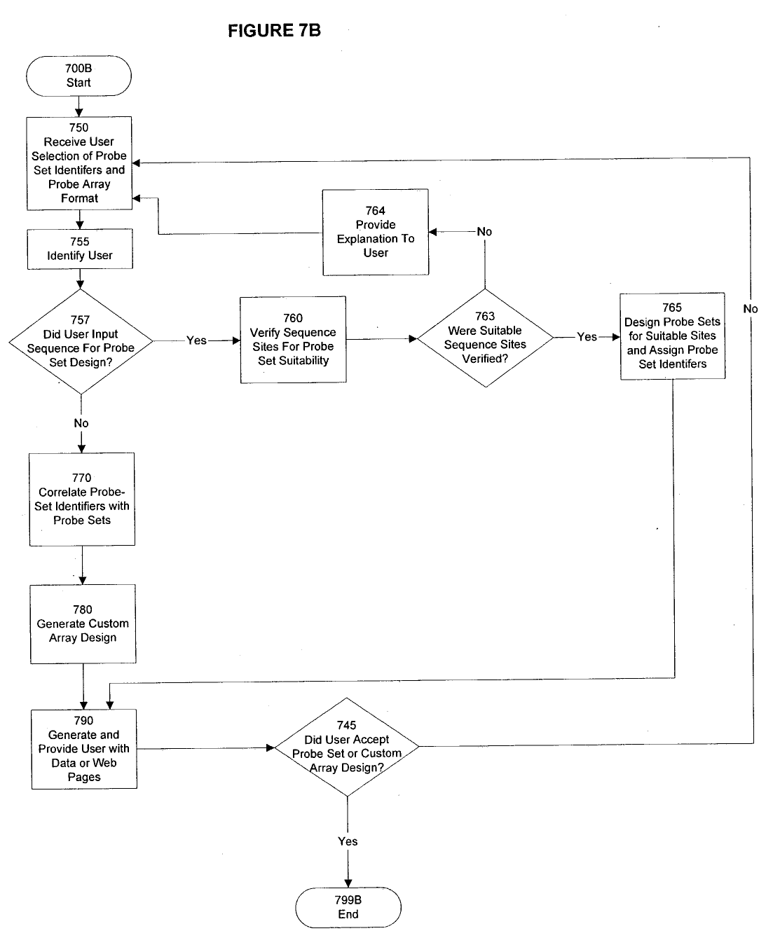

More specific examples are now provided of how user service manager 522 may receive and respond to requests from user 101 for genomic, EST, protein, or annotation information, as well as for product information and/or ordering. These examples are described in relation to FIGS. 7 through 19.

FIGS. 7A and 7B are flow charts representing illustrative methods by which portal 400 may respond to a user's request for genomic information related to alternative splice variants, or a request to provide a customized probe array, respectively. In accordance with step 710 or 750 of these examples, input manager 532 receives from client 410 over Internet 499 a request by user 101. This request may, for instance, include an HTML, XML, or text document (e.g., tab delimited *.txt document) that includes user 101's selection of certain probe-set identifiers. As noted, the probe-set identifiers may be a number, name, accession number, symbol, graphical representation, or nucleotide or other sequence, as non-limiting examples. In some cases, user 101 may make this selection by employing one or more of analysis applications 199A to select probe-set identifiers (e.g., by drawing a loop around dots, selecting portions of a graph or spreadsheet, or other methods as noted above) and then activating communication with portal 400 by any of a variety of known techniques such as right-clicking a mouse. The request may also, in accordance with any of a variety of known techniques, specify that user 101 is interested in genomic and/or product data, as well as details regarding the type of data that is desired. For instance, user 101 may select categories of products, names of vendors or products, and so on from pull-down menus. Manager 532 provides user 101's request to user service manager 522, as described above.

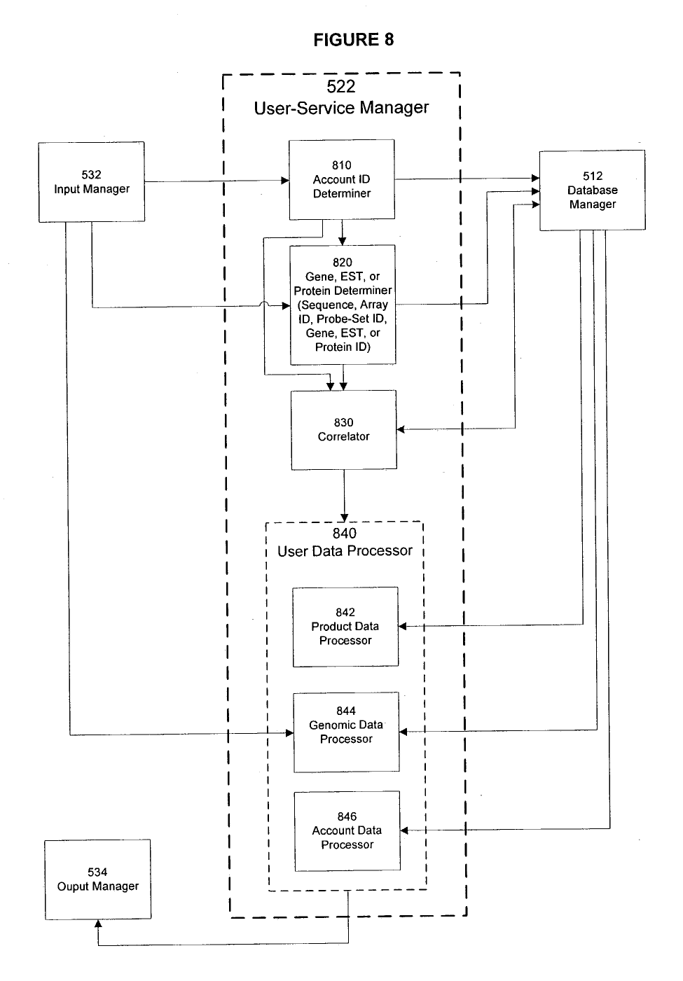

In accordance with step 720, user-service manager 522 initiates an identification of user 101. FIG. 8 is a functional block diagram showing the functional elements of manager 522 in greater detail, including account ID determiner 810 that, in this illustrative implementation, undertakes the task of identifying user 101. Determiner 810 may employ any of various known techniques to obtain this information, such as the use of cookies or the extraction from the user's request of an identification number entered by the user. Determiner 810, through database manager 512, may compare the user's identification with entries in user account database 519 to further identify user 101. In other implementations, the identity of user 101 need not be obtained, although statistics or information regarding user 101 's request may be recorded, as noted above.

In accordance with step 725, user-service manager 522 in one implementation formulates an appropriate query (using, for example, a version of the SQL language) for correlating probe-set identifiers with corresponding genes, ESTs, or proteins. Gene or EST verifier 1110 of Gene or EST determiner 820 is the functional element of manager 522 that executes this task in the illustrated example. Evaluator 1130 forwards the query to database manager 512. If the probe-set identifiers provided by user 101 include sequence information, then the query may seek to verify the existence of one or more corresponding probe sets, consisting of verified probes, from database 511, and/or from SIF information in database 516. If verified, verifier 1110 correlates the identity of the one or more probe sets having a corresponding (e.g., similar in biological significance) sequence with the probe-set identifiers.

If the included sequence information does not have a corresponding probe-set, such as a case in which user 101 has requested to have a probe-set produced, then the included sequence is forwarded to probe sequence verifier/designer 1120. Verifier/designer 1120 performs an analysis of the user-provided input sequence to determine which portions of the sequence should be represented by probes. For example, some portions of the input sequence may consist of short, common repeats that are not effective in uniquely representing the sequence as a whole. Importantly, probes representing the input sequence should be unique, either in combination or, preferably, individually, with respect to the input sequence. To provide a greater statistical likelihood that each probe will be unique, verifier/designer 1120 may require that probes be of a minimum standard length (e.g., 25 bases) that typically may be predetermined based, e.g., on statistical analysis as applied to genomic distributions and various probe-production considerations. However, in some implementations, the probe length may be variable above a minimum determined by verifier/designer 1120 and may be set by the user, or may be selected by the user from a list of permissible options. Verifier/designer 1120 applies various other criteria and tests to verify and/or design probe sequences appropriate for representing the user-provided sequence. For example, verifier/designer 1120 may select or design probes using physical models based upon the thermodynamic properties and uniqueness of the sequence. Elements of the physical models may include energetic parameters (e.g. free energy change □G) derived from each candidate probe sequence, and weight coefficients based upon empirical data. The physical models could include linear regression modeling or other statistical methods used for modeling data.

In some implementations, verifier/designer 1120 may also determine that the user-provided input sequence is not amenable to representation by probes and therefore it will create a report that may be communicated via output manager 534 to the user. For example, verifier/designer 1120 may analyze the complexity of the user-provided sequence and provide the user with a report including a measure of the complexity and a determination that the sequence is insufficiently complex (e.g., it includes too many repeats) to be uniquely and/or reliably represented by a probe set. Additional details regarding the operation of verifier/designer 1120 in alternative implementations are described in U.S. patent applications Ser. Nos. 09/718,295, 09/721,042, 09/745,965, and attorney docket number 3359.3, titled “METHOD AND COMPUTER SOFTWARE PRODUCTS FOR DESIGNING NUCLEIC ACID ARRAYS,” filed Dec. 2, 2002, all of which are hereby incorporated herein by reference in their entireties for all purposes.

It is assumed for convenience with respect to the illustrated implementation of FIG. 11 that verifier/designer 1120 returns the sequence, analysis results, and the one or more probe sequences of one or more probe sets to verifier 1110. Verifier 1110 then formulates an SQL statement specifying the user-provided input sequence, the probe sequences designed by verifier/designer 1120 to represent the input sequence, related analysis results, and possibly other information such as a probe-set identifier for the newly selected probe set. Verifier 1110 directs the SQL statement to database manager 512 in accordance with known techniques so that the information is stored in an appropriate one or more databases, such as database 518 as illustratively shown in FIG. 11. However, as those of ordinary skill in the related art will readily appreciate, there are many other possibilities for routing, processing, and/or storing the data. For example, verifier/designer 1120 could formulate an SQL statement and forward the sequence and analysis results to manager 512 and/or to user data processor 840 to be incorporated into a graphical user interface for presentation to a user. In some implementations, the data need not be stored for later retrieval but simply prepared for display to the user in response to the user's request.

In some implementations, the probe sequences designed by verifier/designer 1120 to represent the input sequence may be used as an identifier for an unknown, e.g., as yet not provided, probe-set. Also, in some implementations, the probe-set identifiers could include one or more terms (e.g. referring to annotation information such as “tumor suppressor”). In either case, user service manager 522 may identify the genes, ESTs, or proteins from database 518, where annotation information is stored with the corresponding genes, ESTs, or proteins. If the probe-set identifiers include names or numbers (e.g., accession numbers), then the query may seek the identity of the probe sets from database 516 that, as noted, includes data that associates names, numbers, and other probe-set identifiers with corresponding genes or ESTs. User 101 may also have locally employed database application 230 to obtain this information, and included it in the information request in accordance with known techniques. In this case, step 725 need not be performed.

In a preferred embodiment, determiner 820 may perform methods for evaluating the presence of alternative splice variants in one or more experiments from an input set of one or more probe-set identifiers and associated hybridization intensities from the one or more experiments. The evaluation methods may be performed by alternative splice variant evaluator 1130, as illustrated in FIG. 11. In one implementation, evaluator 1130 may receive an input set of probe-set identifiers and associated hybridization intensities derived from the results of probe array experiments. Evaluator 1130 performs methods of a kind typically referred to by those-of ordinary skill in the relevant art as “model fitting” to evaluate the probe-set identifiers and associated hybridization intensities for alternative splice variants. For example, evaluator 1130 receives a set of probe-set identifiers and the hybridization intensities associated with each probe-set identifier from a user via input manager 532 (or this same information may be passed to evaluator 1130 via account ID determiner 810 as shown alternatively in FIG. 8). Evaluator 1130 of this implementation formulates a query to database manager 512 to retrieve data related to genomic structure and protein domains based, at least in part, upon the input probe-set identifiers. The genomic structure and protein domain data could for instance include data stored in exon structure or location data 1015, protein-family classification data 1040, or splice-variants data 1020. Evaluator 1130 fits the probe-set identifiers and associated hybridization data to models of known genomic structure of alternative splice variants using, for example, an iterative model-fitting algorithm. For instance, it may be illustratively assumed that a pattern of hybridization data strongly indicates the presence of exons 1 and 3 because probe sets representing those exons have been detected with high intensity values. These data may be taken to indicate that one or more splice variants that include exons 1 and 3 are present. The intensity values related to exons 2 and 4 may, of course, also be relevant to this determination and may change the determination based on the overall best fit of the data. In the present example, each iteration of the algorithm improves the quality of the fit of the data to the known models. One such model, for example, is a linear model that assumes a normal of distribution of variables. It will be apparent to those of ordinary skill in the related art that a variety of different models could be implemented that may also include a variety of assumptions regarding the distribution of variables.

The fit may, in some implementations, be verified using the protein domain data. For example, evaluator 1130 may verify a fit of the probe-set identifier and hybridization intensity data to a model of a particular splice variant by comparing the known function of that splice variant (assuming that there is a known function) to the collective properties of the combined functional domains identified by the data. For instance, the data may identify one or more DNA binding domains that relate to promoter region of a specific gene. Evaluator 1130 may have fit the data to a model of an alternative splice variant that has a known function as a transcription factor of the same gene. In the present example, evaluator 1130 verifies that there is an accurate fit of the data to the model. Additional examples of model fitting and evaluation of alternative splice variants are provided in U.S. patent application Ser. No. 09/697,877 in U.S. Provisional Patent Applications Nos. 60/362,315, 60/362,524, 60/362,454, 60/362,455, 60/362,399, 60/375,351, 60/384,552, 60/398,958, and Attorney Docket No. 3508.1, titled “METHOD OF ANALYZING ALTERNATIVE SPLICING”, filed Oct. 29, 2002, each of which is hereby incorporated by reference herein in its entirety for all purposes.

In the same or alternative implementation, a user may input a set of one or more probe-set identifiers for the purpose of identifying associated alternative splice variants so that the user may design an experiment that may be intended, for example, to confirm that the splice variants are present. For example, evaluator 1130 may formulate a query to database manager 512 to determine alternative splice variants that are known to correspond to the input set of one or more probe-set identifiers provided by the user. Manager 512 retrieves the alternative splice variant data from splice variants data 1020 of local genomic and/or product database 518, or from other databases located locally or remotely. Evaluator 1130 produces alternative splice variant data 1135 from the set of one or more probe-set identifiers and the data retrieved by manager 512 and/or the results from the model fitting methods. Evaluator 1130 then forwards alternative splice variant data 1135 to correlator 830.

As indicated in step 730, user-service manager 522 may then correlate the alternative splice variant data 1135 with the parent gene of each alternative splice variant. Additionally, manager 522 may correlate the indicated genes, ESTs, and/or proteins with genomic, expression, or annotation information as well as product information.

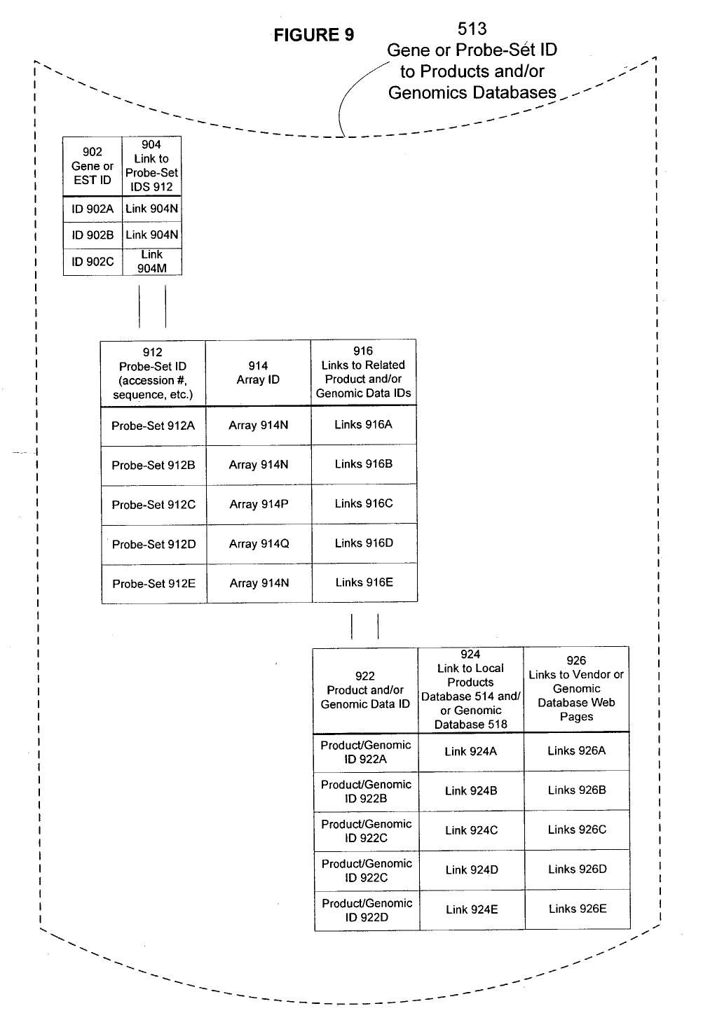

In one of many possible implementations, correlator 830 of manager 522 may undertake this task by formulating a query via database manager 512 to database 513 in order to obtain links to appropriate information in local products database 514 and/or local genomic database 518. FIG. 9 is a simplified graphical representation of database 513. Those of ordinary skill in the art will appreciate that this representation is provided for purposes of clarity of illustration, and that many other implementations are possible. In one aspect of an appropriate query to database 513, which is assumed for illustration to be a relational database, a gene or EST accession number 902 is associated with a link 904 to probe-set ID's 912. As indicated in FIG. 9 by the association of both ID 902A and 902B to the same link 904N, multiple genes and/or ESTs may be associated with the same probe-set ID. The information used to establish these associations is similar to that provided in database 516, as noted above, and the links may thus be predetermined or dynamically determined using database 516.

In other implementations, correlator 830 simply correlates one or more gene or EST identifiers, such as accession numbers, with products, such as biological products. These implementations are indicated in FIG. 8 by the arrow directly from determiner 810 (which is optional) directly to correlator 830. The correlation may be accomplished according to any of a variety of conventional techniques, such as by providing a query to local products database 514, remote pages 404, and/or remote databases 402. These queries may be indexed or keyed by categories, types, names, or vendors of products, such as may be appropriate, for example, in examining look-up tables, relational databases, or other data structures. In addition, the query may, in accordance with techniques known to those of ordinary skill in the relevant art, search for products, product web pages, or other product data sources that are logically or syntactically associated with the gene or EST identifier(s). The results of the query may then be provided by output manager 534 to user 101, such as over Internet 499 to client 410. For example, the genes, ESTs, and/or proteins may include biological sequence information that correlator 830 may correlate with product information that could include probes, probe sets, and/or probe arrays; reagents; instruments for sample preparation, hybridization, incubation, array manufacture, or scanning/signal detection; and so on. In the present example, correlator 830 may determine that there is no product information associated with a biological sequence. Correlator 830 may then return that information to the user via output manager 534 where the user may select the biological sequence for probe set verification and design. Alternatively, correlator 830 may automatically implement the process of sequence verification and probe set design based using the biological sequence. Illustrative methods of sequence analysis for probe set verification and design are described in greater detail below.

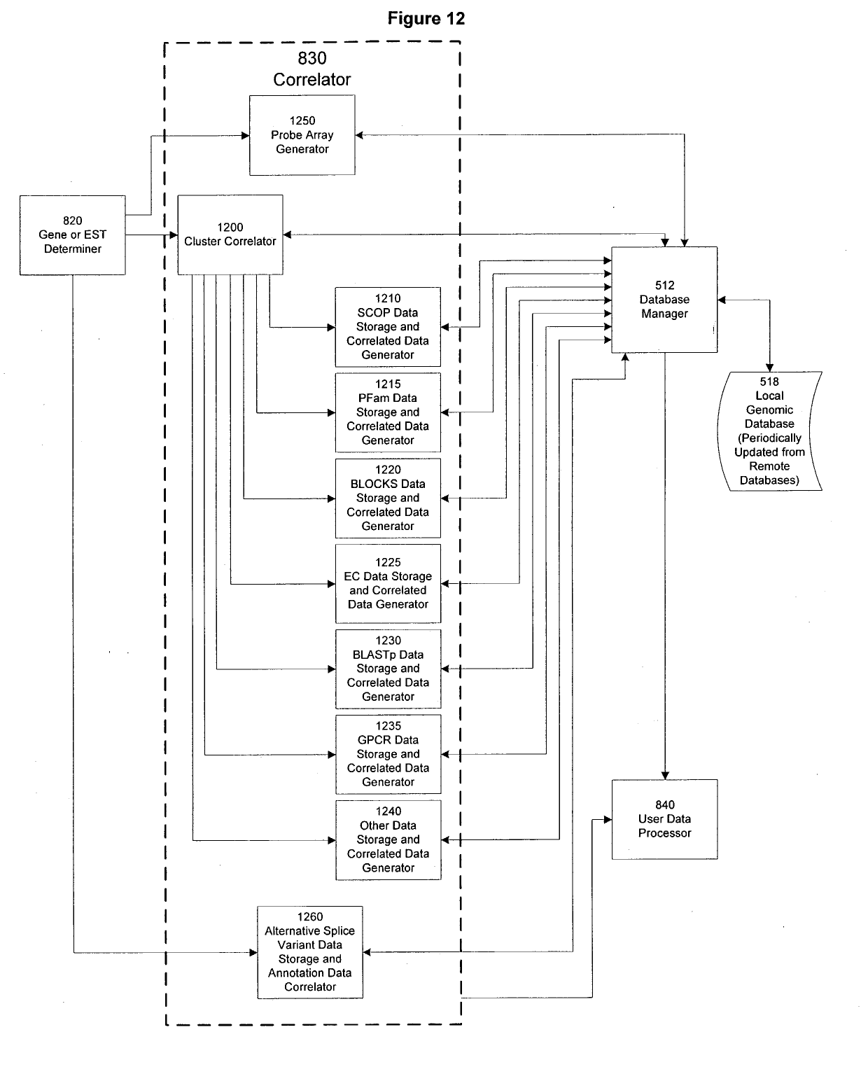

A further implementation of correlator 830 is illustrated in FIG. 12, wherein cluster correlator 1200 receives from gene or EST determiner 820 a nucleotide sequence that may or may not correspond to a probe set. Cluster correlator then correlates the nucleotide sequence via database manager 512 with the corresponding protein sequence found in gene or EST to protein sequence data 1097, as is illustrated in FIG. 10. Alternatively, correlator 1200 may translate the nucleotide sequence into a protein sequence by methods known to those of ordinary skill in the art. Cluster correlator 1200 then sends the protein sequence to data storage and correlated data generators 1210, 1215, 1220, 1225, 1230, 1235, and 1240. The data storage and correlated data generators correspond to databases, now available or that may be developed in the future, that contain information regarding associated protein family, pathway, network, complex, and/or other protein annotation information. Such databases include but are not limited to, SCOP, PFam, BLOCKS, EC, and GPCR, which are known to those in the art as databases that contain annotation information. Such clusters of data may be stored in local genomic and/or product database 518 as illustrated in FIG. 10 as clustering data 1065, 1070, 1075, 1080, 1085, 1090, and 1095. The databases used in this example are for illustration only, and those of ordinary skill in the art know that many other examples are possible.

The data storage and correlated data generators use methods, known to those in the art as clustering methods, to determine sequence or structural similarity and alignments with similar protein sequences and/or structures. There are numerous types of clustering methods used for these purposes, for example what is commonly known as BLASTp represented in FIGS. 10 and 12 as BLASTp clustering data 1085 and BLASTp data storage and correlated data generator 1230. Another example is commonly referred to as the Hidden Markov Model (referred to hereafter as HMM). HMM's are pattern matching algorithms that use a training set of data to “learn” the patterns contained in that training set of data. A preferred implementation is the so-called GRAPA set of HMM's that in the illustrated example are trained to be specific to families of proteins where each family has its own HMM trained to its characteristic pattern. A trained HMM can then analyze a sequence and return a score that corresponds to how well the sequence matches the pattern. In one illustrative implementation, a threshold value is assigned so that a score above the threshold is considered to be a member of the family and a score below is not. The data storage and correlated data generators of this implementation then generate what is commonly referred to as a pairwise alignment between the query sequence and the family consensus sequence, and correlate annotation data corresponding to the family.

Another function of correlator 830 pertains to a user's desire to have a probe array designed from an input set of one or more probe-set identifiers. The functional element of correlator 830 that performs this task in the illustrated implementation is probe array generator 1250. Generator 1250 receives the set of one or more probe-set identifiers and/or the one or more probe sets designed by verifier/designer 1120 from determiner 820. Generator 1250 then produces a probe array design based, at least in part, upon the input set of one or more probe-set identifiers and/or one or more designed probe sets, as well as probe array parameters that could include, for instance, a template or other parameter that could be stored in one or more databases. Generator 1250 of the illustrated implementation assigns a probe array identifier to the designed probe array and forwards that probe array design and associated identifier to database manager 512 for storage in one or more databases that may include local products database 514. Additionally, generator 1250 may forward the probe array design and probe array identifier to user data processor 840 for incorporation into one or more graphical user interfaces. Additional examples regarding the processing and implementation of custom probe array designs are provided in U.S. Provisional Patent Application Serial No. 60/301,298, U.S. Provisional Patent Application Serial No. 60/265,103; and U.S. patent application Ser. No. 09/824,931, incorporated by reference above.

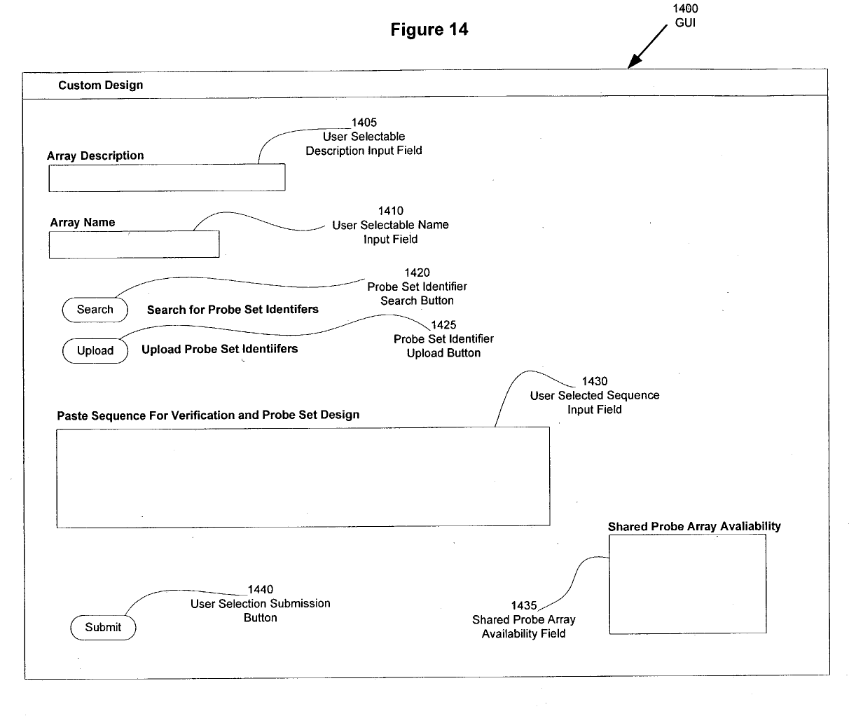

FIG. 7B is a flow chart depicting an illustrative example of receiving a user request for the custom design of probe sets and/or probe arrays. It will be understood that in FIG. 7B, as in FIG. 7A, the particular steps and decision elements, and the sequence and flow indicated among them, are provided as non-limiting examples and that many variations are possible. A graphic example of a web page for executing the user request illustrated in FIG. 7B is presented in FIG. 14 as GUI 1400. In accordance with step 750, a user initiates a request by inputting one or more probe-set identifiers and a selection of probe array format. Alternatively, a default probe array format may be automatically selected. A user may initially name a custom design probe set or probe array by typing or pasting a selected name into user selectable name input field 1410. A user may search for probe-set identifiers by selecting probe-set identifier search button 1420 that could, for example, provide a separate window or pane that displays a list of probe sets or alternatively a list of lists of probe sets where a user may make additional selections to refine the search. Additionally, a user may upload a set of one or more probe-set identifiers by selecting probe-set identifier upload button 1425 that could provide the user with an additional window or pane requesting additional information such as the location of file to upload. A user may also input a sequence by pasting or typing sequence information into user selected sequence input field 1430. The input sequence could be input in a variety of formats including FASTA or other type of format. The user may select a format by typing or pasting array format information into user selectable description input field 1405. In some implementations the format may include a variety of probe array format factors that could be user definable from the input information. Alternatively, a user may input a format identifier into field 1405 to select a predetermined format. In a preferred implementation, the probe array format factors include any one or any combination of the number of probe sets; a shape and/or one or more dimensions of a probe; one or more dimensions of active and/or inactive areas of the probe array; one or more indicators of geographic dispersion of probe sets on the probe array; nominal, maximum and/or minimum number of probes and/or probes in a probe set representing one or more EST, gene, splice variant of a gene, or protein; substrate material or design; and/or design of a hybridization chamber or microfluidics body encompassing and/or associated with the probe array. Additionally, the geographic dispersion of probe sets on the probe array may be defined by the format. The term “geographic dispersion” as used herein refers to the distribution of multiple copies of each probe set across a probe array where the probe sets may, for example, be evenly distributed throughout the probe array. In the same or other preferred embodiment, an even distribution of probe sets may provide a user a degree of protection from a variety of experimental factors that could be detrimental to experimental results. Some of the experimental factors include damage to one or more regions of the probe array or scanner instrument non-linearity. Additionally, an even, or other, distribution of multiple copies of each probe set provides a user with redundancy that may be used in statistical calculations that include accurate confidence levels in the data set.

The user submits the selections and/or input information when the user selection submission button 1440 is selected. In accordance with step 750 of FIG. 7B, the user selections and/or input information is received by gene or EST verifier 1110 and processed as described above.

In accordance with step 755, the user is identified as previously described in relation to step 720 of FIG. 7A. Decision element 757 illustrates whether the user has input a biological sequence for probe set design. In the case in which a user has input a sequence, the sequence is processed in accordance with step 760 by verifying and identifying suitable sequence sites for probe set design as previously described in relation to probe sequence verifier/designer 1120. As illustrated by decision element 763, if sequence sites are verified as suitable for probe set design, then verifier/designer 1120 may design probe sets to the identified suitable sites in accordance with step 765. Alternatively, if there are no verified sites suitable for probe array design then verifier/designer 1120 provides an explanation to the user that could include a GUI. The GUI could include elements of GUI 1500 such as field 1520 to display a text message, and/or elements of GUI 1400 such as input field 1430 to prompt the user to input an additional sequence selection as illustrated in FIG. 7B as the arrow from step 764 to step 750.

In the event that user has input one or more probe-set identifiers for incorporation into a custom probe array, as illustrated by the negative path of decision element 757, then verifier 1110 verifies and correlates the one or more probe-set identifiers with their associated probe sets in accordance with step 770. Verifier 1110 verifies that one or more probes of one or more probe sets exists corresponding to each of the one or more probe-set identifiers and subsequently correlates each verified probe set with its associated probe-set identifier. It will be understood that, in alternative implementations, other methods may be used to enable the user to specify the type, content, arrangement, and other aspects of the probes of a probe array and that the use of either a sequence path or probe-set identifier path in this example is illustrative only.

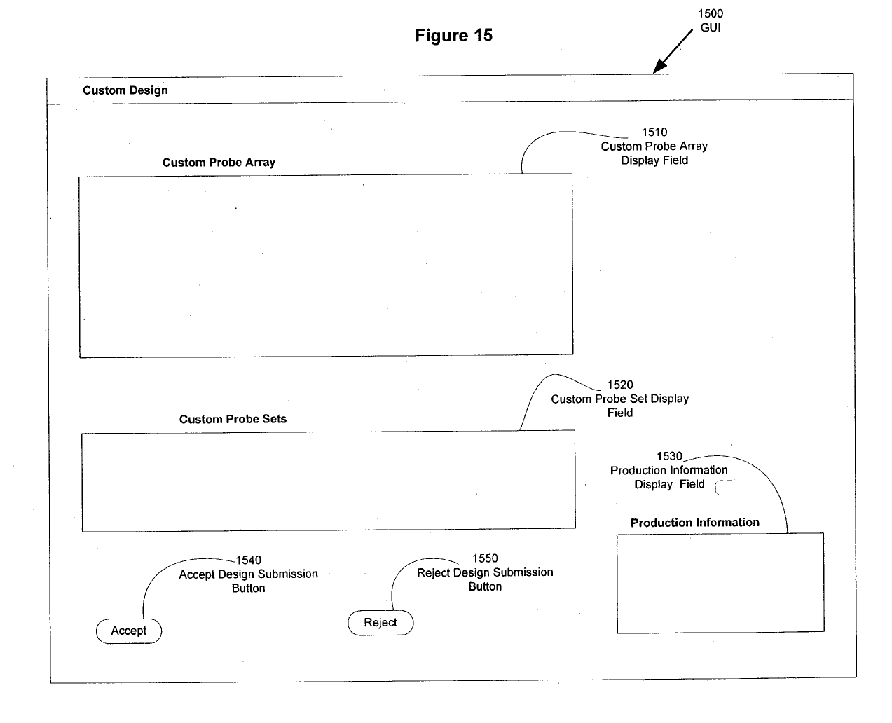

In accordance with step 780, probe array generator 1250 generates the custom probe array design, as previously described, from the associated probe sets and probe array format information. In accordance with step 790, user data processor 840 receives the custom probe array design and/or the custom probe set designs, as well as any associated information and generates a graphical user interface (GUI) as illustrated in FIG. 15 as GUI 1500. GUI 1500 is illustrative and non-limiting as to graphical elements that may be enabled for display by a user using conventional methods for formatting and transmitting such information. It is understood that numerous alternative arrangements and choices of graphical elements are possible. GUI 1500 is enabled to display associated data received by processor 840, such as custom probe array information displayed in custom probe array display field 1510, custom probe set designs displayed in custom probe set display field 1520, and production information display field 1530. Information displayed in illustrative field 1510 may include a list or list of lists of probe-set identifiers, probe array format, date of submission, probe array name, submitter, or other information.

In some implementations, field 1510 may include names of catalog arrays, i.e., arrays previously designed and/or manufactured and stocked for shipment, that include an indicated subset (i.e., an indicated percentage and/or list) of the probe sets of interest to the user. For example, in response to a user's request for design of probe sets or probe arrays corresponding to user-selected probe-set identifiers, gene or EST verifier 1110 determines verified probe sets, as described above. Gene or EST verifier 1110 may also send a query to database manager 512 to determine which of the verified probe sets already exist on catalog arrays, a database of which may be included in local products database 514. Database manager 512 may send information specifying the identified catalog array or arrays to user data processor 840 that may then enable presentation of this information to the user via an appropriate graphical user interface. Thus, for example, the user may be notified by entries in custom probe array display field 1510 that specified probe sets responsive to the user's request are already included in specified catalog arrays. The user may select the identified catalog array and, for example, also select accept button 1540 to indicate a desire to order the selected catalog array. In the manner described below, shipping, price, and other information related to the ordering and shipment of the catalog array may be displayed to the user in production information display field 1530. One of many examples of catalog arrays is the Human Genome U133 Set available from Affymetrix, Inc. Other catalog arrays from Affymetrix are listed, as of November 2002, at http://www.affymetrix.com/products/arrays/index.affx. A custom array may become a catalog array such as, for example, when a user consents that the custom array be made available to other users. Similarly a custom array may become a made-to-order array, which is an array that, like a catalog array, typically is listed for general sale but, unlike a catalog array, typically is not stocked for rapid shipment and instead is made to order.

Even if user 101 orders a catalog array that provides most of the probe sets of interest, the user may decide that it is important to also obtain a custom array including the remaining probe sets of interest (or, of course, decide to obtain a custom array with all of the probe sets of interest). In a case in which user 101 decides to order a custom array having a subset or all of the probe sets of interest, an option in some implementations is to enable the user to order a “shared” probe array that is shared with other users. For example, it is assumed for purposes of illustration only that a custom probe array may be designed for manufacture at a certain price and/or for delivery at a certain schedule with up to 5,000 unique probe sets, which may be referred to as an example of a nominal custom probe set size. A graphical element, such as shared probe array availability field 1435 of GUI 1400, may indicate to user 101 that 4,520 of the 5,000 nominal probe set locations on a newly designed custom array have been sold to, or otherwise reserved for, other users. If user 101 requires 480 or fewer probe sets in a custom array, user 101 may then, for example, select the shared probe array (or arrays) indicated in field 1435 and select submit button 1440, thereby indicating a desire to have the 480 or fewer probe sets included in the selected shared probe array. Optionally, user 101 may select both the shared probe array in field 1435 and one or more of the probe sets specified in field 1430 (prior to verification) and/or field 1520 (subsequent to verification) in order to indicate that only the specified probe sets should be included in the shared probe array. It will be understood that various other methods may be employed in alternative implementations to enable user 101 to specify probe sets that are to be manufactured on one or more probe arrays, some or all of which also contain probe sets manufactured for other users.

In some implementations, a range of probe sets from and including the nominal custom probe set size to a smaller number may trigger production and delivery of the custom array as, for example, if user 101 places an order for 450 probe sets in the instant example. Thus, the “production range of probe sets” for a custom array may be a single value (e.g., the nominal custom probe set size) or a range including a somewhat smaller number of probe sets. User 101 may be informed of this production range when placing the order, or elements of user-service manager 522 (e.g., account data processor 846) may indicate completion of an order for a shared custom array when a total number of orders for probe sets enters the range, with or without informing user 101 of the existence of the range. Thus, a shared probe array may be deemed complete, and ready for production, even though the actual number of probe sets ordered falls short of the nominal custom probe set size. In processing user orders, user-service manager 522 updates an appropriate database in, e.g., local products database 514, to keep track of orders received from various users for probe sets. This operation typically is carried out in cooperation with database manager 512 as noted above. In some implementations, orders for probe sets may be segregated based on various types of probe arrays optimized, or otherwise specially configured, for the particular type of probe sets ordered by the user (e.g., probe sets for gene expression may be produced on arrays of a type different than arrays for genotyping or diagnostics, although such differentiation need not be required in various implementations). Any of a variety of systems (hereafter sometimes referred to as “delivery systems”) may be used to implement delivery and order-fulfillment operations in accordance with techniques for implementing e-commerce, non-limiting examples of which are described in U.S. Provisional Patent Application No. 60/301,298, incorporated by reference above. Similarly, a variety of systems (hereafter sometimes referred to as “production systems”) may be used to produce arrays by, for example, photolithographic techniques as noted above, and also including methods involving direct write optical lithography as described, e.g., in U.S. Pat. No. 6,480,324, which is hereby incorporated herein in its entirety for all purposes. It may therefore be stated herein that genomic web portal 400 of the illustrated implementation may “enable” the user to be provided with probe sets on shared custom arrays (or with catalog arrays in shared lots as described below) by, e.g., providing a production and/or delivery system with the information needed to effectuate production and/or delivery. Alternatively, some implementations may comprise a business entity or other organization or entity, or collection thereof, that includes not just the genomic web portal, but also the production and/or delivery operations and facilities.

In a similar manner, user 101 may share the ordering of a catalog array with other users. For example, prices for probe arrays may be conveniently established in terms of nominal lot size purchases. That is, for example, the purchase of probe array type A in a lot of 200 may cost more than the purchase of the same type A in a lot of 1,000 probe arrays. User 101 may be notified in a graphical element, such as shared probe array availability field 1435, that a probe array of interest to user 101 (as indicated by user 101 by submitting an array name or identifier in field 1410 and/or description in field 1405, or as indicated in field 1435 based on a user's submission of probe-set identifiers) is subject to a lot-sharing arrangement. In such an arrangement, a plurality of users may benefit from sharing a whole-lot order so as to benefit from economies of scale. Thus, for example, user 101 may indicate (e.g., in field 1435) that he or she wishes to purchase 200 type A probe arrays, but is willing to wait until a complete lot of 1,000 type A probe arrays is completed based on the orders of additional users. The graphical user interface may inform user 101 that unfulfilled orders for 700 type A probe arrays have previously been received and thus an order of up to 300 additional type A probe arrays may be accommodated within the pending lot. In some implementations, a range of ordered probe arrays from and including the nominal lot size to a smaller number may trigger production and delivery of the lot as, for example, if user 101 places an order for 250 type A probe arrays, thereby bringing the total to 950 of a nominal 1000 array lot. Thus, the “production-lot range” may be a single value (e.g., the nominal lot size) or a range including lots of a somewhat lesser size (e.g., a range from 950 to 1,000 probe arrays). User 101 may be informed of this production-lot range when placing the order, or elements of user-service manager 522 (e.g., account data processor 846) may indicate completion of a lot when a total number of orders enters the range, with or without informing user 101 of the existence of the range. Thus, a lot may be deemed complete, and thus priced at the rate for the nominal size lot, even though the actual number of orders falls short of the nominal lot size. Of course, user 101 may order a number of type A probes that, alone or together with other unfulfilled orders, exceeds the nominal lot size. In this case, additional lots may be produced, the size of the lot may be increased and thus result in lower costs for user 101 and the other users who have ordered the probe array, or other arrangements may be made. Alternatively, the completed lot may be produced and another pending lot may be created. Thus, in these various implementations, user 101 (and the other users who share the lot) generally may benefit from the lower price corresponding to the larger nominal lot size.

In yet another implementation, user 101 may indicate (e.g., in field 1410) that the user is willing to wait a specified period for the lot to be completed, but withdraws the offer to buy a portion of the lot after a specified date or time. As one of numerous other variations of the shared-ordering option, user 101 may specify that the 200 type A probe arrays should be manufactured on or after a specified date and that user 101 is willing to accept the price at that date based on the number of accumulated orders for type A probe arrays from user 101 and other users. In yet other implementations, user 101 may specify a price (and/or other term of purchase or delivery), i.e., make a bid as in an auction, that user O1 is willing to undertake. Thus, for example, user 101 may specify a willingness to purchase 200 type A probe arrays at a price no greater than X dollars (and possibly also specify that delivery shall occur no later than Y date). If the number of users indicating an interest in purchasing type A probe arrays increases to a level such that economies of scale, or other considerations, result in a lot price for type A probe arrays of X dollars or less, then the order is executed in accordance with conventional techniques for conducting e-commerce (assuming delivery or other terms, if any, also are satisfied). Also, database manager 512 may periodically consult local product database 514 to see if nominal sale prices or lot sizes of probe array type A have been changed so that user 101 's order may be accepted even though the lot size, maximum user-specified price, or other term, had not been met with respect to the original sales price or lot size. In such a manner, discounts, special offers, rewards programs, and so on may be implemented to encourage and efficiently effectuate sales of type A probe arrays.

In one of many possible implementations consistent with various conventional techniques for conducting e-commerce, user-service manager 522 generates production instructions for type A probe arrays to effectuate orders in accordance with any of the preceding, or other, ordering techniques. In some implementations, manager 522 provides the production instructions, e.g., via output manager 534, to a production unit, e.g., one or more of remote vendor business systems and business servers 404. Also in accordance with conventional e-commerce techniques familiar to those of ordinary skill in the relevant art, the type A probe arrays may be shipped directly from one or more businesses corresponding to business servers 404 to user 101 and the other users sharing the production lot, or the probe arrays may be shipped via intermediaries. In some implementations, a business operating genomic portal 400 may undertake production and/or shipping responsibilities, and in some of these implementations servers 404 may be local to portal 400 rather than remote. Further in accordance with conventional e-commerce techniques (or those that may be developed in the future), user-service manager 522 typically records when orders have been fulfilled or various stages of fulfillment reached (e.g., at time of production, shipping, receipt, and so on).

Returning to the description of FIG. 15 and the presentation of information to user 101, illustrative field 1520 may display information including the submitted reference sequence, verified probe set sites, probe set design sequences, date of submission, submitter, or other related information. Illustrative field 1530 may contain detailed production information that includes production schedules, shipping schedules, pricing information, or other type of information related to production and delivery of custom probe sets or custom probe arrays. Fields 1510, 1520 or 1530, or various elements displayed in them, may be combined or separately displayed in numerous configurations. GUI 1500 is presented to the user via a network so that, in some implementations, the user may select to accept or reject the custom probe array design and/or the custom sets designs according to decision element 745. If the user reject any or all of the designs by selecting reject design submission button 1550, GUI 1400 may be displayed so that the user may elect to re-input information according to step 750. GUI 1400 in alternative implementations may include explanations of why a user-input sequence, or selection of probe-set identifiers, were not included in the design of a probe array. If the user accepts a valid design, the use may select accept design submission button 1540 to accept the designs that may then be processed as a customer order, as described further below.

An additional implementation of correlator 830 includes receiving alternative splice variant data 1135 from determiner 820. Data 1135 is illustratively shown as received and processed by alternative splice variant data storage and annotation data correlator 1260. Correlator 1260 formulates a query to database manger 512 to find genomic structure and protein domain information, based at least in part upon data 1135. In some implementations, for example, correlator 1260 in this manner retrieves information that includes genomic structural domains, functional protein domains, and translation frame for each alternative splice variant contained in data 1135. Correlator 1260 also, in some implementations, may determine the overall putative function of each alternative splice variant, based at least in part upon the composition of the genomic structural domains, the functional protein domains, and the translation frame. For example, correlator 1260 may accomplish this function by identifying particular regions of genomic structure, such as for instance a characteristic seven transmembrane domains, and/or the functional protein domains, such as one or more receptor domains. In the present example, the alternative splice variant may be identified as a cell surface receptor by the presence of the seven transmembrane and one or more receptor domains. Correlator 1260 may forward data 1135, genomic structural domain, functional protein domain, and putative function domain to database manager 512 for storage in one or more databases, as well as to user data processor 840 for incorporation into one or more graphical user interfaces for presentation to a user.

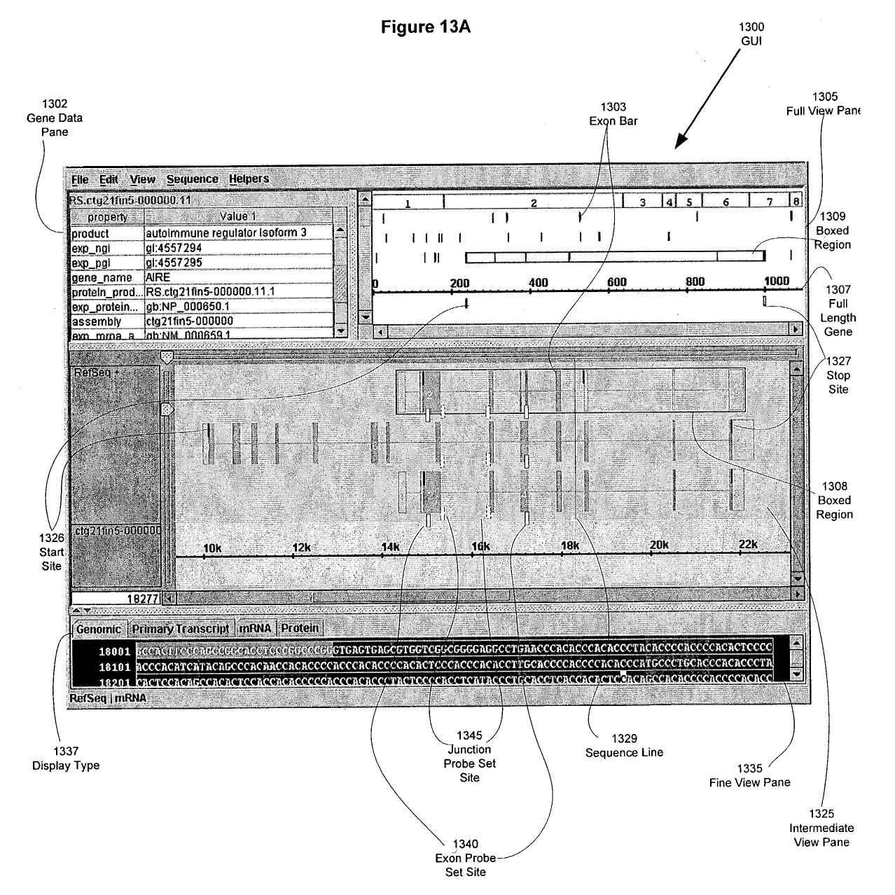

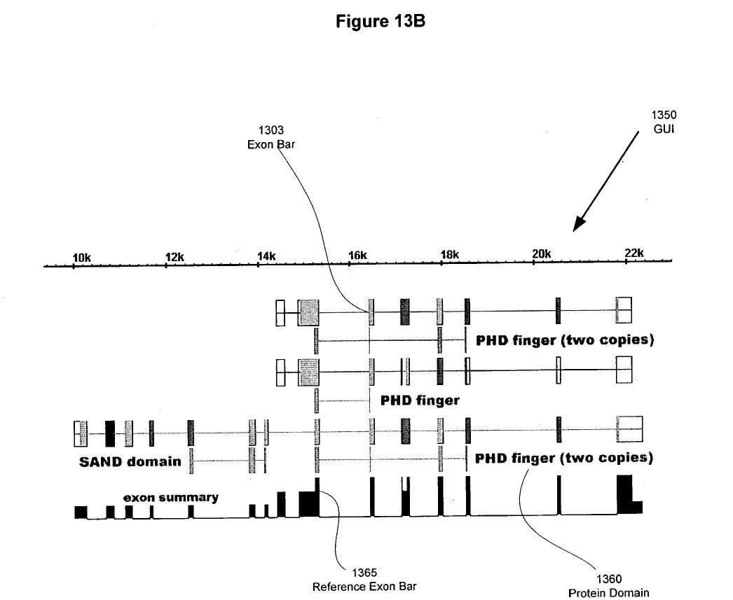

FIGS. 13A and 13B are representations of illustrative examples of graphical user interfaces providing user 101 with information obtained by evaluating one or more probe-set identifiers for alternative splice variants. It will be appreciated by those of ordinary skill in the relevant art that numerous alternative formats, both textual and graphical, may be used in other implementations. FIG. 13A illustrates GUI 1300 that includes a variety of individual panes. It will be understood that the illustrated combination of panes is non-limiting and that, in various implementations, the panes may be otherwise combined or separately displayed by themselves (i.e., in a single graphical user interface). Also, not all of the panes need be displayed or available in all implementations. In the illustrated example, gene data pane 1302 presents information relating to the gene from which the alternative splice variants are derived. Such information could include gene name, protein name, accession numbers, protein ID numbers, splice variants ID's, numbers of variants, variant function, as well as other related genomic and/or experimental information. In some implementations, pane 1302 may display information related specifically to a splice variant selected by the user. This selection may include, for example, selection of an exon or region in panes 1305, 1325, or 1335 (described below) by pointing of a mouse or other technique, by selection of a probe-set identifier corresponding to the splice variant, by providing a sequence of interest, and by other methods. The information in pane 1302 may include links to local and/or remote databases or resources such as, for example, by hyperlink to genomic information over the Internet.

Full view pane 1305 displays full length gene 1307 as a scale of the number of bases, where the exon regions of alternative splice variants are aligned along the scale. The gene represented in this manner may have been selected by a user in accordance with any of the techniques noted herein. In this implementation, each variant is distinguished from the others by displaying the variant along a separate horizontal line, i.e., by separating the variants vertically in pane 1305. However, it will be understood that many other graphical arrangements or devices known to those of skill in the art may be used to distinguish splice variants and/or distinguish exons belonging to one or more splice variants. For example, the variants and/or their exons may be color-coded, identified by differently shaped objects, and so on.

A user may wish to view a particular splice variant, or a particular region of a splice variant, in greater detail. In one example of how the user may indicate this wish, the user may select boxed region 1309 by, for example, dragging a mouse along the horizontal representation of the bottom horizontal display of exons in pane 1305 that represents the arrangement of exons in a particular splice variant. Alternatively, the user could click on the start and end of the desired region, enter a chromosomal location as may in some implementations be indicated by the scale at the bottom of pane 1305, enter a location relative to the beginning of the gene or relative to another marker like a SNP site, enter a sequence to specify a start and/or stop area, and so on. The resulting expanded view of boxed region 1309 is displayed as boxed region 1308 in intermediate view pane 1325.

In addition to providing an expanded view of a user-selected splice variant or portion thereof, intermediate view pane 1325 in the illustrated example displays additional alternative splice variants aligned to one another and to a full length reference sequence. Displayed in both full view pane 1305 and intermediate view pane 1325 are start site 1326 and stop site 1327. Site 1326 may indicate the start site of transcription and/or translation and site 1327 may represent the site of termination of transcription and/or translation. Also displayed in pane 1325 is exon probe set sites 1340 and junction probe set sites 1345 that are illustrative examples of probe set annotations. Sites 1340 represent the regions of exons that are interrogated by probe sets, and similarly sites 1345 displays the relationship of probe sets that interrogate the junction region where two exons may be spliced together. In the illustrated implementation, each of the displayed boxes of sites 1340 may represent a single probe set whereas each of the displayed boxes of sites 1345 may represent a portion of a probe set that may, for instance, include a box representing half a probe set that interrogates the sequence region at the end of one exon (e.g., the 5′ end) and another box representing the remaining half of the probe set may interrogate the sequence at the end of another exon (e.g., the 3′ end). In some implementations it is not necessary that adjacent boxes of sites 1345 belong to the same probe set, rather each box may be representative of some portion of a probe set that may be used in combination with a box belonging to sites 1345 representing a complementary portion. For example, a box belonging to sites 1345 at the 5′ end of exon one may represent a portion of a probe set that could, for instance, be half the number of probes of a probe set. A complimentary box could be located at the 3′ end of exon two, three, or the 3′ end of any exon contained within a particular gene that contains the remaining portion of a probe set that identifies a splice variant containing exon one spliced to exon two, three, or other exon defined by the probe set.

The relative abundance of alternative splice variants may also be displayed in panes 1305 and 1325. Methods for representing abundance may include color coding of exon bars 1303, variations in exon bar height, variations in exon bar pattern, or other graphical methods commonly used to distinguish differences. The measure of abundance could include the relative expression level of each alternative splice variant, the frequency of exon usage in all alternative splice variants, or other user-selected measure. For example, illustrated in FIG. 13B is GUI 1350 that includes reference exon bar 1365. A user may cause GUI 1350 to be displayed by, for example, clicking on the “Protein” tab that is shown illustratively in fine view pane 1335 of FIG. 13A. GUI 1350 may, in such an implementation, be displayed in place of sequence line 1329 in fine view pane 1335. The height of exon bar 1365 may correspond, as one of the examples noted above, to the frequency with which an exon, or partial exon, occurs in the alternative splice transcripts. In the present example, various bar heights may occur within each exon and between different exons.

In the illustrated implementation shown in FIG. 13A, sequence line 1329 in pane 1325 indicates the region of sequence that is displayed in fine view pane 1335. Fine view pane 1335 may include a region of sequence that is user selectable. That is, a user may move line 1329 by any of a number of known techniques, such as dragging it with a mouse, and thereby indicate the region of sequence that is to be displayed in pane 1335. Also, the user may select the type of sequence viewed in pane 1335 in some implementations. The type of sequence may include the genomic DNA sequence, primary RNA transcript, messenger RNA transcript, and/or translated protein sequence. The user may also select to view one or more of the alternative splice transcripts and/or full length reference sequence aligned together.

Each of the panes of GUI 1300 in the illustrated implementation has what are referred by those in the related art as scroll bars. A user may interact with GUI 1300 by selecting a scroll bar and moving it in a desired direction to change what is displayed in the associated pane. For example, a user may select the vertical scroll bar associated with fine view pane 1335 and move it in a desired direction. The displayed sequence displayed in pane 1335 will change according to the direction of movement of the scroll bar as well as the position of sequence line 1329 in intermediate view pane 1325.

Additionally, a scroll bar or other method of selection could be used for what may be referred to as “semantic zooming”. This term as used herein refers to increasing or decreasing the levels of magnification and resolution in a display. With a change in magnification, objects may change appearance or shape as they change size. Moreover, when magnification of a displayed image is increased, additional information may be displayed relating to elements of the display. Conversely, when the magnification of an image is decreased, less information may be displayed for individual elements of the display. For example, when alternative splice variants are displayed at low magnification, the displayed image may include general exon structure and alignments. As the magnification is increased, the sequence of the alternative splice variants may be displayed as well as annotation information. Thus, not only is the magnification of the information changed, the amount, content, and/or type of information also may be changed in relation to the change of magnification. For a review of semantic and other zooming technology, see, e.g., CounterPoint: Creating Jazzy Interactive Presentations, Good, L., Bederson, B. B., HCIL-2001-3, CS-TR-4225, UMIACS-TR-2001-14, March 2001; Jazz: An Extensible Zoomable User Interface Graphics Toolkit in Java, Bederson, B., Meyer, J., Good, L. HCIL-2000-13, CS-TR-4137, UMIACS-TR-2000-30, May 2000, In ACM UIST 2000, pp. 171-180; Jazz: An Extensible 2D+Zooming Graphics Toolkit in Java Bederson, B., McAlister, B. HCIL-99-07, CS-TR-4015, UMIACS-TR-99-24, May 1999; Does Zooming Improve Image Browsing? Combs, T., T. A., and Bederson, B., HCIL-99-05, CS-TR-3995, UMIACS-TR-99-14, February 1999 In ACM Digital Library Conference, pp. 130-137; Graphical Multiscale Web Histories: A Study of PadPrints Hightower, R. R., Ring, L. T., Helfman, J. I., Bederson, B. B., and Hollan, J. D. ACM Conference on Hypertext 1999; Does Animation Help Users Build Mental Maps of Spatial Information, Bederson, B. and Boltman, A., CS-TR-3964, UMIACS-TR-98-73, September 1998, In IEEE Info Vis 99, pp. 28-35; A Zooming Web Browser, Bederson, B. B., Hollan, J. D., Stewart, J., Rogers, D., Vick, D., Ring, L. T., Grose, E., Forsythe, C. Human Factors in Web Development, Eds. Ratner, Grose, and Forsythe, Lawrence Erlbaum Assoc., pp 255-266, 1998; Implementing a Zooming User Interface: Experience Building Pad++, Bederson, B., Meyer, J., Software: Practice and Experience, 28 (10), pp. 1101-1135, August 1998; When Two Hands Are Better Than One:Enhancing Collaboration Using Single Display Groupware, Stewart, J., Raybourn, E. M., Bederson, B. B., Drum, A., ACM CHI 98 Summary, 1998; KidPad: A Design Collaboration Between Children, Technologists, and Educators, Druin, A., Stewart, J., Proft, D., Bederson, B. B., Hollan, J. D., ACM CHI 97, pp 463-470, 1997; A Multiscale Narrative: Gray Matters, Wardrip-Fruin, N., Meyer, J., Perlin, J., Bederson, B. B., Hollan, J. D.,ACM SIGGRAPH 97 Visual Proceedings, p 141, 1997; A Zooming Web Browser, Bederson, B. B., Hollan, J. D., Stewart, J., Rogers, D., Druin, A., and Vick, D. SPIE Multimedia Computing and Networking, Volume 2667, pp 260-271, 1996; Local Tools: An Alternative to Tool Palettes, Bederson, B. B., Hollan, J. D., Drum, A., Stewart, J., Rogers, D., Proft, D., ACM UIST '96, pp 169-170, 1996; Pad++: A Zoomable Graphical Sketchpad for Exploring Alternate Interface Physics, Bederson, B., Hollan, J., Perlin, K., Meyer, J., Bacon, D., and Fumas, G., Journal of Visual Languages and Computing, 7, 3-31, 1996, HTML, Postscript without pictures (74K), PDF without pictures (77K) 1995; Space-Scale Diagrams: Understanding Multiscale Interfaces, Furnas, G., Bederson, B., ACM SIGCHI '95; Advances in the Pad++Zoomable Graphics Widget, Bederson, B., Hollan, J. USENIX Tcl/Tk'95 Workshop; Pad++: Advances in Multiscale Interfaces, Bederson, B. B., Stead, L., Hollan, J. D. ACM SIGCHI '94 (short paper), 1994; Pad++: A Zooming Graphical Interface for Exploring Alternate Interface Physics, Bederson, B. B., Hollan, J. D.,, ACM UIST '94, 1994; Pad—An Alternative Approach to the Computer Interface, Perlin, K., Fox, D., ACM SIGGRAPH '93; A Multiscale Approach to Interactive Display Organization, Perlin, K., Coordination Theory and Collaboration Technology Workshop, National Science Foundation, June 1991, each of which is hereby incorporated by reference herein in their entireties for all purposes.

Additional interactive features of GUI 1300 may include selecting elements such as an exon bar 1303 by moving a cursor via mouse or keyboard and clicking the button on the mouse, or pressing the enter key on the keyboard, or other method commonly used for selecting elements. When a user selects an element or elements, portal 400 may alter the display in the graphical user interface and/or present one or more additional graphical user interfaces, or windows. One such example of interactive features is presented in FIG. 13B. GUI 1350 shown in FIG. 13B displays alternative splice variants in the context of what is referred to as the “central dogma” of molecular biology. The central dogma generally refers to the process of DNA producing transcripts (RNA) that are translated into protein. For example, a GUI may be used to display the alternative splice transcripts by level (i.e., DNA, RNA or protein) as indicated by GUI 1350 that presents information at the protein level. Similar to FIG. 13A, FIG. 13B displays exon bars 1303 and other related elements. Additionally, GUI 1350 may display additional elements such as protein domain 1360. As is well known to those of ordinary skill in the relevant art, proteins often include conserved regions referred to as domains, or modules, that have distinct evolutionary origin and function. Information regarding the sequences, locations, homology, functions, two-dimensional or three-dimensional structure, and other aspects of protein domains or modules may, for example, be obtained in the manner described above from numerous remote databases 402 (for example, the Smart, Pfam, and NCBI CDD web-based databases and similar databases that may be developed in the future). Additional aspects of data collection and characterization regarding protein domains and protein-protein interactions are described in U.S. Provisional Patent Application No. 60/385,626, filed Jun. 4, 2002, titled “System, Method, and Product for Predicting Protein Interactions,” which is hereby incorporated herein by reference in its entirety for all purposes. The elements displayed in GUI 1350 may vary according to the level. For example, the protein level may display protein annotations in greater detail than the transcript level, while the transcript level may display greater detail with respect to probe set annotations. Additional examples of visualizing alternative splice variants are provided in U.S. Provisional Patent Applications Nos. 60/394,574 and 60/375,875, incorporated by reference above.

As used herein, the term “graphical user interface” is intended to be broadly interpreted so as to include various ways of communicating information to, and obtaining information from, a user, which need not be a human being. For example, information may be sent to a user in an email as an alternative to, or in addition to, presenting the information on a computer screen employing graphical elements (such as shown illustratively in FIGS. 13A and 13B). As is known by those of ordinary skill in the relevant art, the email may include graphics, or be designed to invoke graphics, similar to those that may be displayed in an interactive graphical user interface.

One of many possible examples of the utility of these features includes a situation in which user 101 inputs a nucleotide sequence for which there is no corresponding existing probe set, as determined by correlator 830, described above. The sequence is translated by correlator 830 into a protein sequence by known methods (alternatively, user 101 may have entered a protein sequence), and clustered using the HMM's for all, or any user-selected portion, of the available databases. In the present example, it is assumed that a number of positive family identifications are made, and all related annotation data is presented to the user via a GUI as well as being stored on the user's LIMS system. After compiling and reviewing the annotation data, the user may choose to order a probe set that corresponds to the nucleotide sequence by including the new probe set in an order for a custom probe array.

In yet another example, a user may specify a sequence, which may for example be a putative gene that does not correspond to any probe set. Correlator 830 correlates the user-specified sequence with one or more of the databases shown in FIG. 10 (or other databases) included in database 518, and identifies possibly related sequences (which may be related by family, functional, or other criteria other than, or in addition to, sequence). User-service manager 522 identifies the probe sets associated with the related sequences and/or the associated ESTs, genes, and/or proteins. The identified probe sets, and optionally the array types in which they are represented, are provided to user 101 in an appropriate GUI and/or by other techniques such as email. Examples of probe-set annotations are provided in U.S. Provisional Patent Application Serial No. 60/306,033, incorporated by reference above.

Returning to the example of FIG. 9 described above, correlator 830 formulates a query via database manager 512 to database 513 to obtain links to appropriate information located in local products database 514 and/or local genomic database 518. With respect to some specific implementations, one or more links 916 to related products and/or genomic data may be obtained by following the appropriate links 904 to probe-set ID's 912. In the present example, link 904N may link to probe-set 912C, which is associated with links 916C to related product and/or genomic data. The information used to establish this association may be predetermined based on expert input and/or computer-implemented analysis (e.g., statistical and/or by an adaptive system such as a neural network) of the nature of inquiries by users. For example, it may be observed or anticipated (by humans or computers, as noted) that users conducting gene expression experiments resulting in the identification of certain genes may wish to use antibodies against the genes to conduct follow-on protein level experiments. The association between the genes and the appropriate antibodies may be stored in an appropriate database, such as database 516. Links 916C may thus include links to product or genomic data identifiers that identify links to data about the appropriate antibodies (for example, a link to product/genomic ID 922A), to catalogues of antibodies generally (e.g., ID 922B), or to a probe array specifically designed for detecting alternatively spliced forms of the genes of interest (e.g., ID 922C). It is assumed for illustrative purposes that, in a particular aspect of this example, link 916C leads to ID 922C. Information about the availability of splice-variant probe arrays may be predetermined by the contents of links 926. For example, links 926D (associated with ID 922C, as shown) may be stored Internet and/or database-query URL's leading to vendor web pages, local products database 514, and/or local genomic database 518. Also, the content of links 926D may be dynamically determined by query of databases 514 or 518 or of remote data sources such as databases 402 or web pages 404. These and similar processes are represented by step 735 of FIG. 7.

As will now be appreciated by those of ordinary skill in the art, numerous variations and alternative implementations of this illustrative arrangement of database 513 are possible. For example, probe-set identification data may be linked to array identifiers (such as array ID 914), which may then be associated with links 916. As another of many possible examples, gene or EST accession numbers may be linked directly to product and/or genomic data ID 922 or, even more directly, to links 926. Implementations such as the illustrated one provide opportunities for making broad associations based on a more narrow inquiry by a user. For instance, a user may select only one probe-set identifier, but that identifier may be linked to multiple genes and/or ESTs, which may be linked to multiple products or genomic data. In another example, link 926D may include a link to local genomic database 518. Based on the probe-set identifiers, gene or EST accession numbers, sequence information, or other data provided by or deduced from user 101's inquiry, database 518 may be searched for associated data in accordance with known query and/or search techniques.

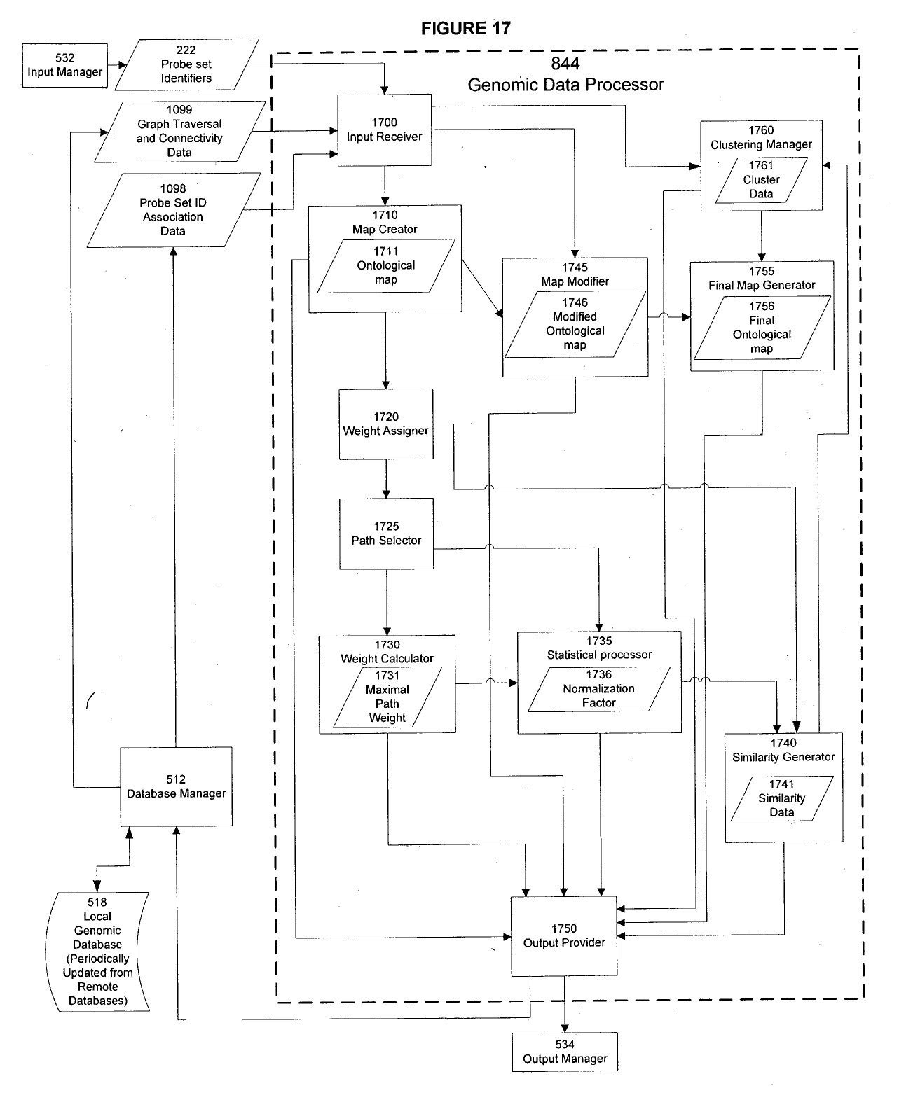

Returning now to FIG. 7A and step 740 in particular, data returned in accordance with the query posed by correlator 830 may be provided to either product data processor 842, genomic data processor 844, or both, as appropriate in view of the nature of the returned data. The functions of processors 842 and 844 are shown as separated for convenience of illustration, but it need not be so. Processors 842 and 844 apply any of a variety of known presentation or data transfer techniques to prepare graphical user interfaces, files for transfer, and other forms of data. This processed data is then provided to output manager 534 for transmission to client 410. Further details of the function of processor 844 will become evident in view of the following examples and with respect to the description of FIG. 17 below.

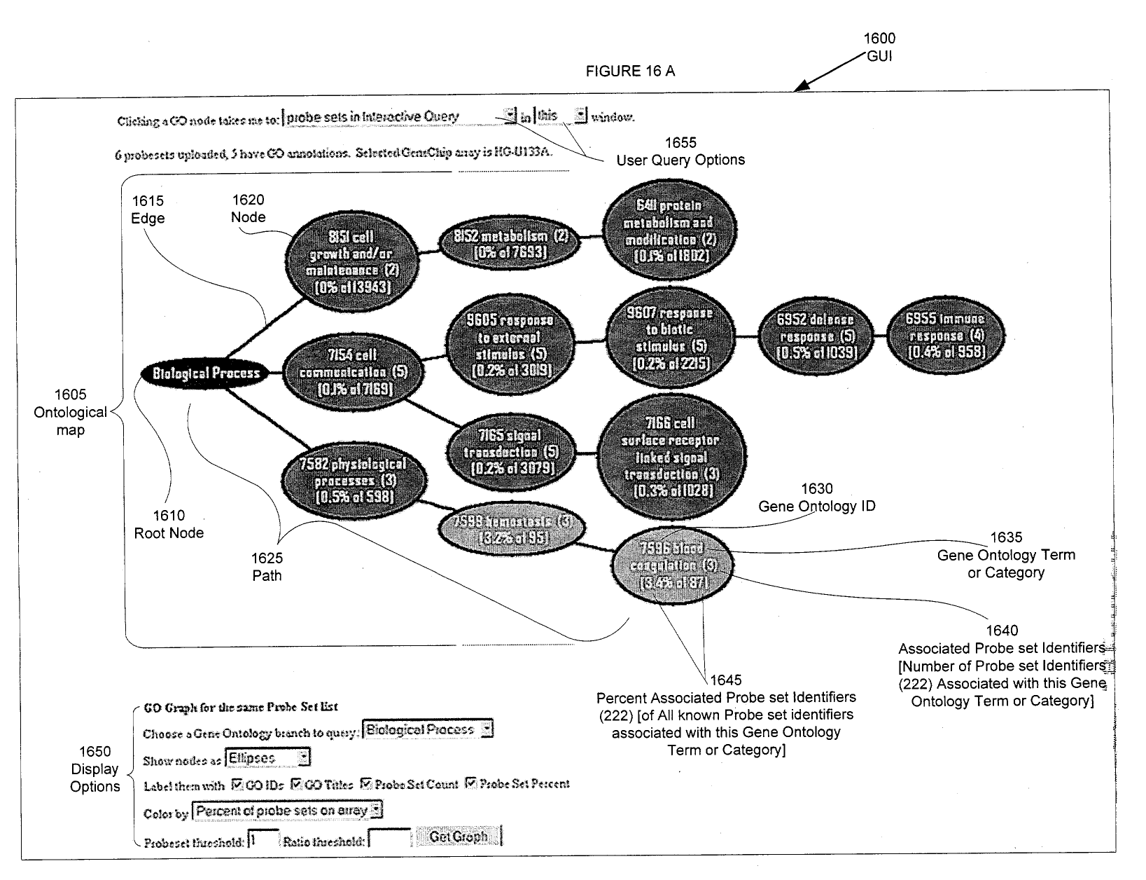

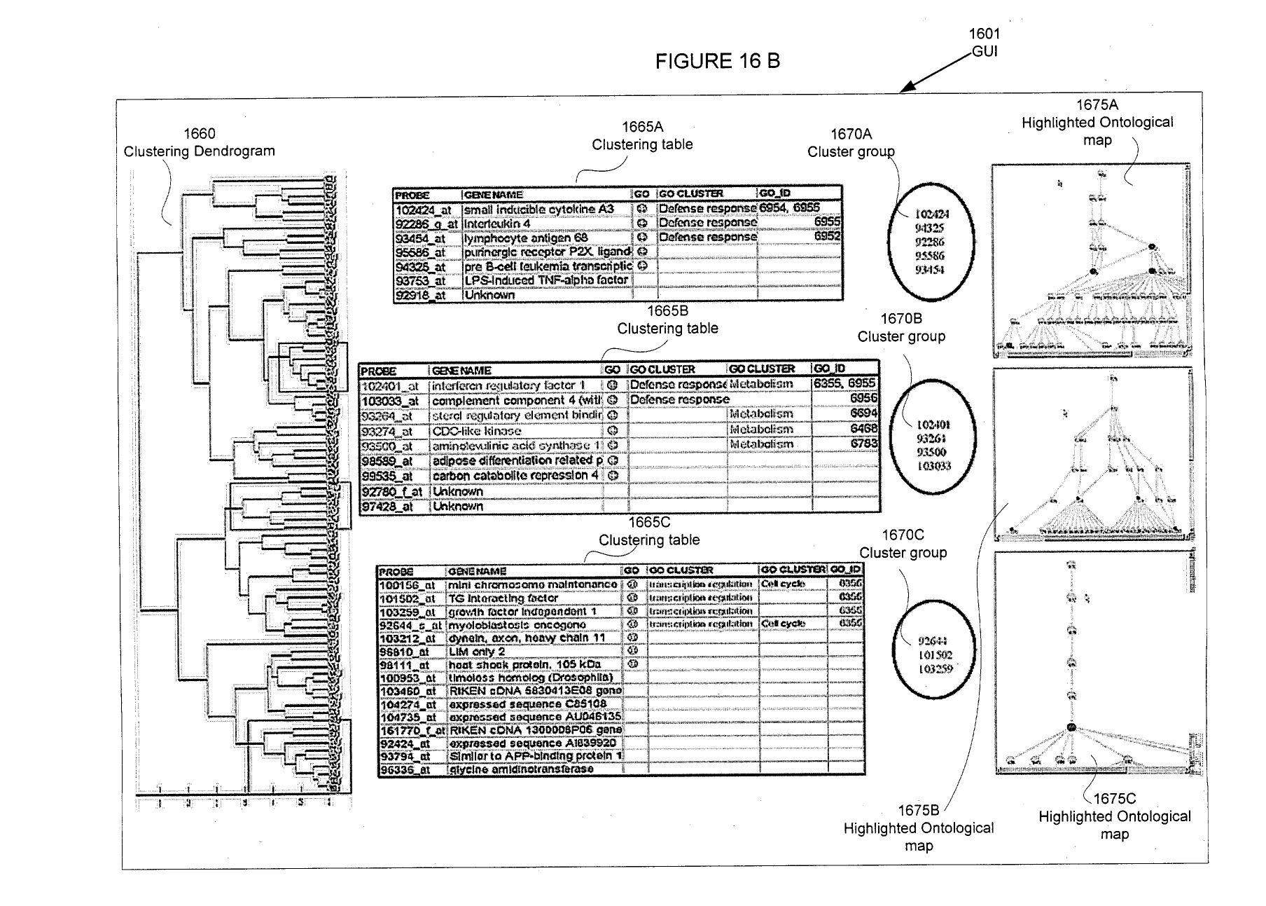

FIG. 16A is a representation of an illustrative example of a graphical user interface providing user 101 with information generated by genomic data processor 844. In GUI 1600, the provided information includes ontological map 1605 comprising a plurality of nodes such as node 1620, one or more edges such as edge 1615, one or more paths such as path 1625, and one root node 1610 from which all other nodes, all edges and all paths originate. Path 1625 includes a plurality of nodes with each node being connected to another node by an edge.

For convenience of illustration, ontological map 1605 and other aspects of the present invention are described herein with reference to a particular widely used scheme and program, developed and maintained by the Gene Ontology™ (GO) Consortium, for providing biological knowledge and genetic ontological information in particular. Biological knowledge, as used herein, refers to information that describes function (e.g., at molecular, cellular and system levels), structure, pathological roles, toxicological implications, and so on. It will be understood that although the GO system is illustratively referred to herein, various other systems for providing biological knowledge and genetic ontological information, such as the MGED Ontology system, may be employed in alternative implementations. At the core of the GO system is a dynamic controlled vocabulary for molecular biology that may be applied to all organisms and may be updated as biological information accumulates and changes. Further information about GO may be found in Gene Ontology: tool for the unification of biology, Nature Genet. 25: 25-29 (the Gene Ontology Consortium, 2000). Access to this ontological system, and information about it, are currently available over the Internet at http://www.geneontology.org/.

In accordance with the GO scheme of the illustrated implementation, there are three major categories of terms: biological processes, molecular function, and cellular component. In accordance with the current scheme, a gene may be annotated with several GO terms, which may be in one or more of the major categories. For example, the Degenerin gene is annotated with “peripheral nervous system development,” “monovalent inorganic cation transport,” “central nervous system development,” and “synaptic transmission” within the category of biological process; is annotated with “amiloride-sensitive sodium channel” within the category of molecular function; and is annotated with the term “integral plasma membrane protein” within the category of cellular component. Gene annotations using GO terms, and similar annotations using other schemes and organizations, provide an excellent resource for retrieving and associating summarized biological knowledge. For example with respect to GO, genes with similar biological properties are annotated with the same or similar terms, and thus may easily be identified and associated.

GO terms are structured as a digraph, meaning that a term may have multiple parents and/or multiple children thus allowing what may be referred to herein as “multiple inheritance.” Considering each GO term as a node in the digraph, two connected nodes defines an edge. A collection of connected edges defines a “path”. For instance, the term “protein kinase” may have two parents: “kinase” and “phosphotransferase”, and five children: “protein histidine kinase,” “protein serine/threonine kinase,” “protein threonine/tyrosine kinase,” “protein tyrosine kinase,” and “transmembrane receptor protein kinase.” The closer a GO term is to the root of the digraph, the more general the biological classification is. The relative position of two nodes in the digraph reflects the biological distance between the two terms. For example, if protein tyrosine kinase and protein histidine kinase are both child nodes of protein kinases, they share higher biological relevance then with another term that has a different parent node, for example amiloride-sensitive sodium channel. Genes annotated with protein tyrosine kinase and protein histidine kinase, respectively, are functionally similar since they both fall in the category of protein kinase. Thus, the more common ancestors two GO terms share in their paths, the more similar they are biologically.

Current analytical methods involving GO primarily use GO terms as a keyword system where genes annotated with the same GO terms are recognized. This approach has not taken the full advantage of GO since relationships between GO terms are not captured. In fact, genes annotated with different GO terms may be closer then genes annotated with the same GO term, depending on the geographic positions of the GO terms. For example, two proteins annotated with JUN_kinase and SAP-kinase respectively may have higher similarity to each other based upon existing facts then the two genes both annotated with the same term phosphotransferase, because phosphotransferase is a more general term and is closer to the root. Thus, using GO annotations without considering the overall digraph structure can be misleading. Tracing the topological locations of GO terms in the GO digraph allows one to assess the biological relationship among terms accurately and thus assess more sensitively the biological similarity between genes.

In some embodiments, the methods of the invention transform the biological knowledge captured in GO into a numeric form based upon the digraph structure. A pair-wise similarity matrix between GO terms is generated based upon the relative positions of GO terms in GO paths. This matrix is used to specify similarity measurements between two genes based upon gene annotations using GO terms. Since one gene may have multiple GO annotations, a greedy approach may be used by taking the highest scoring pair of GO terms for two genes to assign the pair-wise similarity score. Additional details are provided below with respect to genomic data processor 844.

In accordance with the GO scheme of the illustrated implementation, each edge represents the relationship of “is a” or “is a part of,” meaning that a child node is either a part of the parent or is a more specific example of the parent term. Therefore, the closer a term is to the root, the more general is its biological classification. Similar to phylogeny classifications, the deeper the edge in a path, the finer or more resolved the classification. For example, it may illustratively be assumed that “molecular function” has child nodes including “transcription regulator” and “transporter,” and that “transporter” has child nodes including “ion transporter” and “lipid transporter.” Further, “lipid transporter” has child nodes including “fatty acid transporter” and “aminophospholipid transporter.” The similarity between two nodes that share a 3-edge partial path “molecular function—transporter-lipid transporter—aminophospholipid transporter” is higher than two nodes sharing a 2-edge partial path “molecular function-transporter-lipid transporter.” And since the classification gets finer toward the leaf (i.e., away from the root), the edge “lipid transporter—aminophospholipid transporter” is assumed to have less added similarity value than “transporter-lipid transporter.” It will be understood, however, that in alternative implementations other weighting schemes could be used to implement systems for comparing nodes, e.g., for calculating values in a greedy algorithm, as long as a consistent approach is employed. For example, in another implementation, it could alternatively be assumed that edges away from the root provide more added similarity value.

As illustrated in FIG. 16A, ontological map 1605 may be based upon, and may be changed by, one or more user selections of display options 1650 as described below. In this illustrative example, map 1605 pertaining to “Biological Process” has been selected for display, wherein the user input includes specification of certain probe-set identifiers 222. Other maps such as those pertaining to “Molecular Function” or “Cellular Component” may be similarly selected for display. As used in this context of providing ontological maps, the word “display,” and grammatical variations thereof, are to be interpreted broadly to include not just display as on a display monitor, but also the provision of data to a user (human or machine) in accordance with any other conventional technique (e.g., by printing, sending email, producing audible signals, and so on) or other techniques that may be developed in the future for providing data to users. The word “display,” and grammatical variations thereof, are similarly broadly intended in other contexts such as of probe sets or probe sequences aligned with biological sequences (including other probe sequences), described below.

The geometric shape of node 1620 in graph 1605 may be user defined by selection of options 1650. The geometric shape could include an elliptical shape rectangular shape, polygonal shape, or other type of geometric shape. Node 1620 may display a variety of information based upon display options 1650; for example, a Gene Ontology identifier (ID) 1630 (e.g., “7956”), an associated Gene Ontology category or term 1635 (e.g., “blood coagulation”), associated probe-set identifiers 1640 (representing the number of probe-set identifiers in the group of identifiers 222 selected by the user that are associated with ID 1630 or term 1635), and percent associated probe-set identifiers 1645 (the ratio of identifiers 1640 to the total number of all known probe-set identifiers associated with ID 1630 or term 1635). In one implementation the total number of probe-set identifiers may comprise, for example, those identifiers specifically associated with probe sets of one or more Affymetrix® GeneChip® probe arrays.

In the illustrated implementation, map creator 1710, described below, determines the values for identifiers 1640 and percentage 1645. Map creator 1710 may also associate a node, e.g., node 1620, with other information not necessarily pertaining to probe-set identifiers 222 (for example LocusLink IDs, Unigene ID, Swissprot ID, and so on). Thus, as those of ordinary skill in the related art will now appreciate, map 1605 may be used as a generalized tool for visual data mining by associating nodes, edges and/or paths with other information including one or more probes or probes set sequences of a custom probe set, genomic sequences, alternative splice variants, ESTs, RNAs, mRNAs, gene regions, genes or chromosomes. For example, the nodes and edges may be used to display the relationships between the proteins in a metabolic pathway. Those relationships may include functional, structural, and/or evolutionary relationships. As will now be appreciated by those of ordinary skill in the art in view of the present disclosure, various similar applications are possible.

The broad applicability of this invention will be appreciated from the fact that ontological map 1605 need not necessarily be restricted to displaying ontological information. Map 1605, or numerous variations thereof making use of the functional elements and/or method steps described herein for generating and displaying the map, may be used to display an association of one or more of probes, probe sets, and/or probe arrays; reagents;-instruments for sample preparation, hybridization, incubation, array manufacture, or scanning/signal detection; and so on, with information regarding their characteristics or properties. In an illustrative example, map 1605, or a variation thereof, may display an association of one or more reagents to the probe arrays, wherein the nature of association represents the use of a reagent in an experiment conducted with a probe array. Furthermore, in another illustrative example, map 1605, or variations thereof, may display an association of one or more probe set identifiers, probe sets or probe arrays to one or more diseases or disease states, wherein the nature of association represents a presence or absence of probes associated with the probe set identifiers, probe sets or probe arrays in the said diseases or disease states.

In yet another illustrative example, map 1605 or variations thereof may display an association of one or more probe set identifiers, probe sets or probe arrays to regulatory cascades and/or pathways including regulatory pathways involving, e.g., DNA methylation and histone deacetylation, wherein the nature of association represents the regulation including change in the level or amount of one or more biological sequences identified by probes of the probe set identifiers, probe sets or probe arrays.

In yet another illustrative example, map 1605 or variations thereof may display an association of one or more probe set identifiers, probe sets or probe arrays to other one or more probe set identifiers, probe sets or probe arrays, wherein the nature of association includes an evolutionary nature. As is well known to those of ordinary skill in the art evolution is defined as a biological phenomenon in which the gene pool of a population gradually (usually over millions of years) changes in response to environmental pressures, natural selection, and genetic mutations. For example, a first probe identified by a probe set identifier of a probe set of a probe array may be an ancestor (in the context of evolution) of one or more other probes. This association may be displayed in the form of map 1605 or variations thereof, with root node 1610 displaying the first probe and one or more nodes 1620 displaying the probes associated in an evolutionary nature with the first probe. The preceding example is solely for illustrative purposes and is not to be interpreted as limiting or restrictive in any manner whatsoever.

In yet another illustrative example map 1605 or variations thereof may display an association of one or more probes, identified by probe set identifiers of probe sets of probe arrays, with one or more types of RNAs including but not limited to mRNAs, snRNAs, or miRNAs, wherein the nature of this association represents presence or absence of similarity of the probes with the one or more types of RNAs. As is known to those of ordinary skill in the relevant art, small nuclear RNA (snRNA) is the name used to refer to a number of small RNA molecules found in the nucleus. These RNA molecules are important in a number of processes including RNA splicing and maintenance of the telomeres i.e. the chromosome ends. Similarly, micro RNA (miRNA) is the name used for the small RNA molecules believed to play a role in regulating expression of genes.

In a preferred implementation, map 1605 is interactive. For instance, a user selection of node 1620 and user query options 1655 may initiate a further query of one or more databases to obtain (e.g., display and/or download) information pertaining to ID 1630 and/or term or category 1635. Similarly, a user may elect to obtain further information regarding only those of probe-set identifiers 222 found to be associated with the node and/or all of the known probe-set identifiers known to be associated with the node. The obtained information may be displayed in a variety of formats that could include both textual and graphical formats based, at least in part, upon user query options 1655. For example, user query options 1655 may include an option to display associated probe-set identifiers 1640 in an interactive query, in a textual list, in various other Gene Ontology viewers (for example the AmiGO viewer of the Gene Ontology consortium), or in a new ontological map 1605 with the selected node 1620 being displayed as the root node in the new map.

Additionally, in the same or other implementations, terms or categories 1635 associated with the various nodes 1620 comprising map 1605 may be listed in a textual format enabling them to be indexed or searched by various methods known to those of ordinary skill in the art, for example by regular expression search or search using synonyms. Similarly, all other information associated with the various nodes 1620 comprising map 1605, for example ID 1630, term or category 1635, those probe-set identifiers 222 found to be associated with the node, and/or all the probe-set identifiers known to be associated with the node, may be displayed in a textual format in the same or a different graphical user interface.

Various display options 1650 may be selected by user 101 to change the appearance of map 1605. For example, in some implementations, the nodes, edges and paths in map 1605 may be colored dependent upon various user-selected or predetermined parameters, including a threshold value of the associated probe-set identifiers 1640 and/or the percent associated probe-set identifiers 1645. In an illustrative example, the nodes having associated probe-set identifiers 1640, or percent associated probe-set identifiers 1645, above a threshold value may be colored red, and those below the threshold value may be colored blue. Alternatively, one or more colors may be used in manner similar to their use in a heat map. As is well known to those of ordinary skill in the art, a heat map is used to display data by applying colors that vary over a range depending on data value. For example, data having a value between 1 and 10 may be colored in a range of colors from blue to red, with data having a value of 1 being colored blue, data having the value 10 being colored red, and data lying between 1 and 10 being colored in transitions of color from blue to red. Such a scheme is conventionally applied, for example, to display gene expression values in a heat map that is graphically associated with a dendrogram for purposes of clustering genes and/or experiments or experimental conditions. (See, e.g., U.S. Pat. No. 6,223,127, hereby incorporated by reference herein in its entirety for all purposes.) Many other color ranges and criteria for coloring are possible. Similarly, the choice of threshold value may determine whether or not a node will be displayed in map 1605. For example, choosing a threshold value of zero in some implementations would result in a map having all the nodes in the relevant GO scheme, i.e., in the relevant Gene Ontology Directed Acyclic Graph, whereas in other implementations the converse may be true, i.e. choosing a threshold value of zero would result in a map having none of the nodes in the relevant Gene Ontology Directed Acyclic Graph.

Moreover, the appearance of map 1605 may be changed by user 101 through various other parameters including, but not limited to, assigning a predetermined or user-selected weight to determine or change the color of the nodes and/or edges. As is known to those of ordinary skill in the art, the term “weight” can mean a number, a factor assigned to a number in a computation, or various other mathematical or statistical values. Other examples of the parameters that may be used to determine appearance may include expression intensity or signal data from one or more microarray experiments, including experiments involving one or more Affymetrix® GeneChip® probe arrays. Yet other parameters include various statistical factors and measurements such as those taking into account probe-set-to-gene redundancy or variations in the size of map 1605, and one or more similarity metrics between probe-sets pertaining to different Gene Ontology maps such as “Molecular Function”, “Cellular Component” and “Biological Process.”

Additionally in the same or other implementations, map 1605 may be used to compare the results of a plurality of microarray experiments. For example, one or more ontological maps such as map 1605 generated for various sets of probe-set identifiers 222 in a plurality of experiments may be colored differently to illustrate the difference between the expression, presence, structure, or composition of various genes in the plurality of microarray experiments. Additionally, the nodes, edges and the paths may be highlighted in different ways, in the same or one or more new ontological maps, thus illustrating differences across the plurality of microarray experiments. Moreover, differences in gene expression presence, structure, or composition across various probe-set identifiers 222 within the same microarray experiment may be illustrated in a similar manner in the same or one or more new ontological maps. Moreover, it will be understood that many other graphical arrangements or devices known to those of skill in the relevant art may be used to highlight the nodes, the edges and the paths. For example, the nodes, edges and paths may be color-coded, identified by differently shaped objects, sized according to differences in gene expression across various probe-set identifiers 222, and so on.

In yet another embodiment, ontological map 1605 may be represented in a GUI that allows user 101 to interact with map 1605 in a three-dimensional manner. User 101 may move map 1605 in three-dimensions that includes zooming in and out, moving up or down, and/or rotating the map in all directions of motion. In such an implementation, user 101 may be afforded a three-dimensional view of map 1605 to facilitate interaction in three dimensions. User 101 may interact with map 1605 through a variety of devices that facilitate the three-dimensional interaction so afforded. These devices, as is well known to those of ordinary skill in the art, could include but are not limited to, one or more immersive visualization environments, virtual-reality displays, haptic devices, haptic displays, force-feedback devices, tactile feedback devices, trackballs, light pointers, laser pointers, speech interfaces, and numerous other devices facilitating multi-modal human-computer interaction.

Ontological map 1605 may be used to display various other types of networks or hierarchies not necessarily pertaining to the ontology of the Gene Ontology Consortium. For example, it may be used as a general map viewer for protein-protein interactions, drug-protein interactions, various signaling pathways, protein family hierarchies or pathway traversal, and interaction data from GenMAPP and numerous databases including but not limited to DIP and BIND. In addition, the expression intensity or signal data from one or more microarray experiments may be combined with all of the above to further aid in data mining and data visualization.

FIG. 16B is a representation of an illustrative example of another graphical user interface providing user 101 with information generated by genomic data processor 844. In the preferred implementation, probe-set identifiers 222 are clustered by genomic data processor 844 in accordance with techniques described below. The clustered probe-set identifiers 222 may be displayed in one or more clustering dendrogram 1660, one or more clustering tables 1665A, 1665B and 1665C (generally and collectively referred to as tables 1665), and/or as one or more cluster groups 1670A, 1670B and 1670C (cluster groups 1670). Clustering tables 1665 may display various information pertaining to the probe-set identifiers 222 that have been clustered into cluster groups 1670. Furthermore, as indicated in clustering tables 1665 of this implementation, cluster groups 1670 contain probe-set identifiers 222 associated with the same Gene Ontology category or term. The displayed information may include names, associated genes and Gene Ontology term or category, and Gene Ontology ID associated with the probe-set identifiers 222 included in the cluster groups 1670. It will be understood that the illustration is non-limiting and that, in various implementations, the various tables, clusters, and cluster groups may be otherwise combined or separately displayed by themselves (i.e., in a single graphical user interface). Also, not all of the tables, clusters and cluster groups need be displayed or available in all implementations.

The one or more highlighted ontological maps 1675A, 1675B and 1675C (maps 1675), including such map as ontological map 1605 illustrated in FIG. 16A, may display the nodes that are associated with Gene Ontology IDs or Gene Ontology terms associated with probe-set identifiers 222 in one or more of cluster groups 1670, colored differently from the other nodes in the map or highlighted in some other manner.

Clustering dendrogram 1660, one or more of clustering tables 1665, one or more of cluster groups 1670, and one or more of highlighted ontological maps 1675 may be selected by user 101 for displaying further information. In one illustrative example, clicking on a probe-set identifier 222 in clustering dendrogram 1660, in one or more of clustering tables 1665, or in a cluster group 1670 may lead to display of annotation information pertaining to the clicked probe-set identifier 222 in the same or a different user interface. In another example, clicking on a node in one or more of maps 1675 may display various information as describe above for FIG. 16A, in the same or a different user interface. It will be appreciated by those of ordinary skill in the relevant art that the same information may be displayed in numerous additional formats, both textual and graphical (e.g., connected node diagrams, dendrograms, heat maps, and so on) in other implementations.

In some implementations, user 101 may respond to the data thus transmitted by indicating a desire to purchase a product or receive further information. A request for further information may be processed in a manner similar to that described above and illustrated in FIG. 7A as decision element 745. If user 101 indicates a desire to purchase a product, the indicated product may be prepared for shipment or otherwise processed, and the user's account may be adjusted, in accordance with known techniques for conducting e-commerce. As one of many alternative implementations, user-service manager 522 may notify the product vendor of user 101's order and the vendor may ship, or order the shipment of, the product. Manager 522 may then note, in one aspect of this implementation, that a fee should be collected from the vendor for the referral.

In some implementations of portal 400, user 101 may provide to portal 400 (e.g., via client 410, Internet 499, and input manager 532) one or more gene or EST accession numbers or other gene or EST identifiers. Alternatively, or in addition, user 101 may provide to portal 400 one or more probe-set identifiers. User 101 may obtain the gene, EST, and/or probe-set identifier from a public source, from notations user 101 has taken as a result of experiments with a probe array or otherwise, from a list of genes or ESTs having corresponding probes on a probe array, or from any other source or obtained in any other manner. Input manager 532 receives the one or more gene, EST, or probe-set identifiers and provides it or them to user-service manager 522, which formulates a query to database manager 512. In accordance with known query techniques and formats, the query seeks information from local products database 514 of product information related to the gene, EST, and/or probe-set identifiers. For this purpose, local products database 514 may be indexed, or otherwise searchable, for products based or keyed on any one or more of gene, EST, and/or probe-set identifiers. Some implementations may include, according to known techniques, similarity matching of a gene, EST, or probe-set identifier if, for example, all or part of a gene, EST, SIF (corresponding to the probe-set identifier) sequence is submitted. Also, a name-association function, in accordance with known techniques such as look-up tables, may be performed so that alternative names or forms of a gene, EST, or probe-set identifier may be found and used in the product data inquiry. In addition, in some implementations, manager 522 may initiate a remote data search of remote databases 402 and/or remote vendor web pages 404, in accordance with known Internet search techniques, to obtain product information from remote sources. These searches may be based, for example, on product categories or vendors associated in local products database 514 with products, categories, or vendors associated with the gene, EST, or probe-set identifier provided by user 101. Manager 522 may provide product data corresponding to the gene, EST, and/or probe-set identifier, obtained from local products database 514 and/or remote pages or databases 404 or 402, and provide this product data to user 101 via output manager 534. For example, this product data may be included in web pages 524. In some of these implementations, portal 400 thus provides a system for providing product data, typically biological product data. The system includes input manager 532 that receives from user 101 one or more of a gene, EST, and/or probe-set identifier; user-service manager 522 that correlates the gene, EST, and/or probe-set identifier with one or more product data and that causes (e.g., via database manager 512) the product data to be obtained either locally from, e.g., database 514 or, in some implementations, remotely from, e.g., pages 404 or databases 402; and output manager 534 that provides the product data to user 101.

Similarly, a method is provided for providing biological product data, including the steps of receiving from user 101 any one or more of a gene, EST, and/or probe-set identifier; correlating the gene, EST, and/or probe-set identifier with one or more product data; causing the product data to be obtained either locally from, e.g., database 514 and/or remotely from, e.g., pages 404 or databases 402; and providing the product data to user 101.

Returning now to genomic data processor 844, further details of its operation in an illustrative implementation are now described in relation to FIG. 17. Additional details of a possible implementation are provided in U.S. Patent Application Attorney Docket No. 3545, incorporated herein by reference above. As shown in FIG. 17, processor 844 may include input receiver 1700, map creator 1710, weight assigner 1720, path selector 1725, weight calculator 1730, statistical processor 1735, similarity generator 1740, map modifier 1745, clustering manager 1760, final map generator 1755 and output provider 1750.

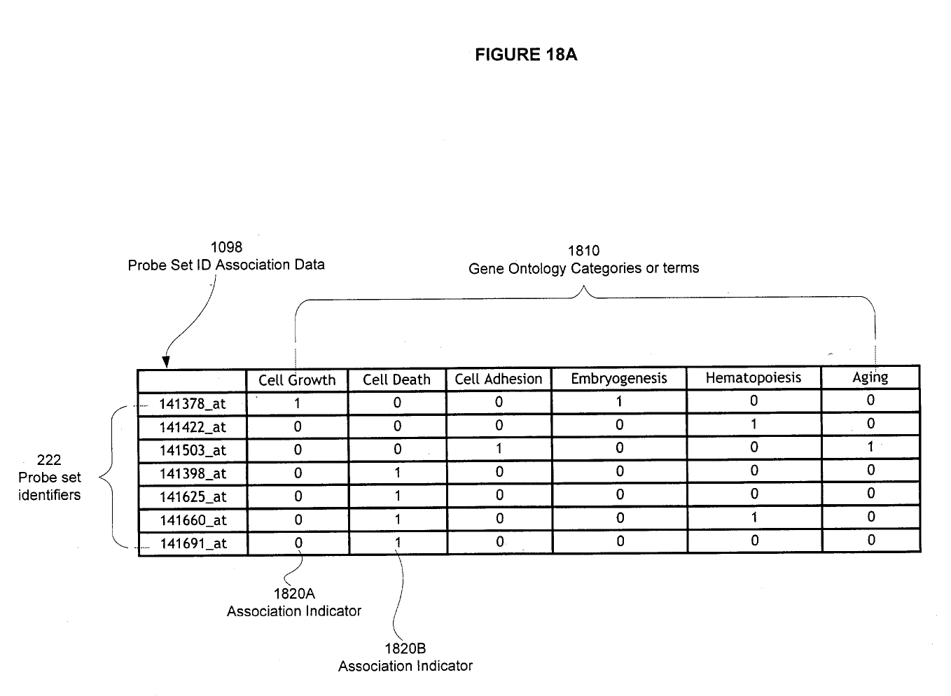

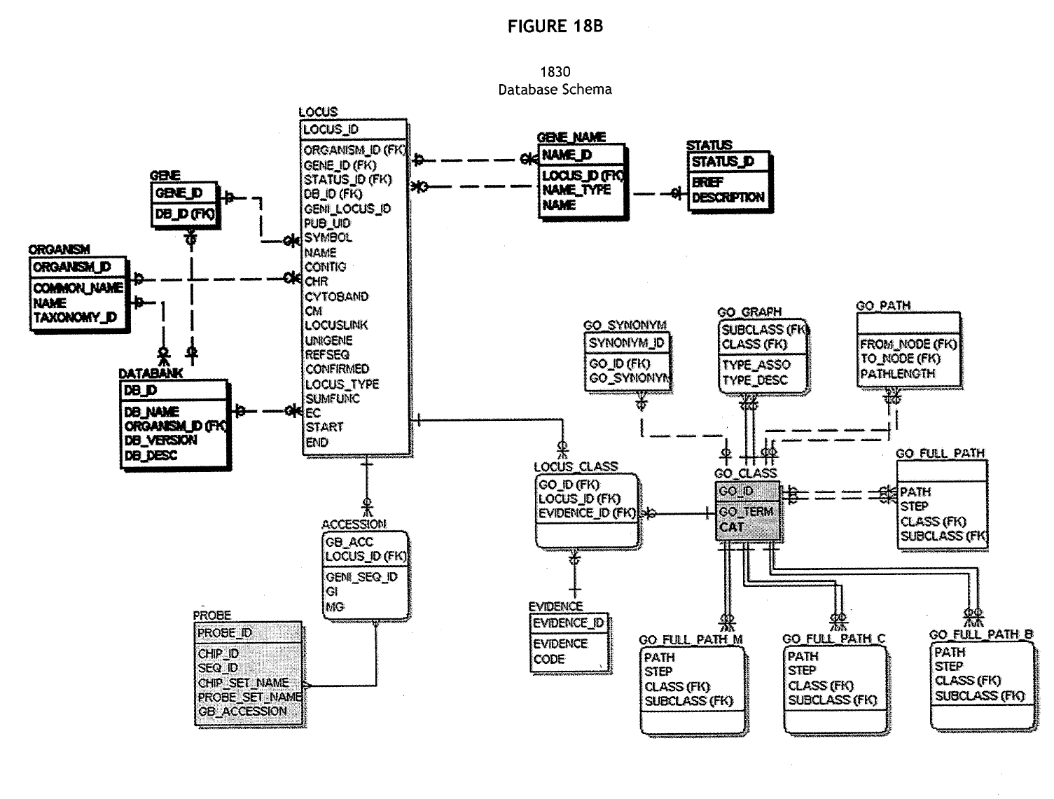

Input receiver 1700 receives and processes, in accordance with conventional techniques, a variety of genomic data including probe-set identifiers 222, graph traversal and connectivity data 1099, and probe-set identifier association data 1098 (displayed in detail in FIG. 18A). Graph traversal and connectivity data 1099 may include data pertaining to genetic ontological maps and, for example, connectivity of Gene Ontology categories and terms. As noted, a Gene Ontology map such as may be represented by map 1605 in some implementations may consist of nodes, edges and paths. As also noted, each Gene Ontology category or term may be represented by a node in the map, each node may be connected to one or more nodes by means of a connector or link called the edge, and a collection of interconnecting edges and nodes comprises a path. The one node in the map from which all other nodes directly or indirectly originate, is known as the root node. Employing conventional graphical manipulation and display techniques, map creator 1710 creates ontological map 1711 that is based on the correlation of the Gene Ontology categories or terms included in graph traversal and connectivity data 1099 and probe-set identifier association data 1098.

Map modifier 1745 modifies ontological map 1711, based on probe-set identifiers 222, to generate modified ontological map 1746. The various modifications of ontological map 1711 could include coloring the nodes associated with the probe-set identifiers 222 in a different color form other nodes or any other method to highlight the nodes, as described above.

Weight assigner 1720 assigns weights to the edges in the relationship-map 1711 based, in one implementation, on the number of intervening nodes and edges lying between the root node and an edge. The weights may be assigned in inverse relation to the number of intervening nodes and edges between an edge and the root node; thus the fewer the intervening nodes and edges, the higher are the assigned weights in this implementation. In addition, the weights assigned to the edges may include a numerical value that allows detailed statistical calculations to be performed. For example in one implementation, the edges may be assigned numerical weights based on the equation, Wt=(1.5)^{-n/2} where Wt is the weight assigned to an edge and n=(level-1), the level of the root node is zero and increases numerically by one for every subsequent node through the relationship-map 1711.

Path selector 1725 chooses a path between any two nodes in ontological map 1711 such that the path consists of the minimum possible number of intervening edges and nodes, such a path being called the shortest path. In an illustrative example the shortest path may include the path between a node and the root node, or the path between any two nodes that may include one or more other nodes and edges connecting the two nodes to the root node. Moreover, path selector 1725 also chooses a longest or maximal path between the root node and another node in the relationship-map 1711 consisting in this implementation of the maximum possible number of intervening nodes and edges.

Weight calculator 1730 calculates a maximal path weight 1731 based on the weights assigned to the edges included in the maximal path selected by path selector 1725. This task may be accomplished in some implementation by adding the assigned weights, or by various other statistical methods known to those of ordinary skill in the art. For example, in one implementation, the maximal path weight denoted by ‘C’ may be calculated by the formula:

$$C = {\sum }_{i = 0}^{∞} {( Wt )}^{i} . ,$$

where (Wt)ⁱ represents the weight of an edge ‘i’ in the maximal path, thus C represents the sum of the weights of the edges comprising the maximal path.

Statistical processor 1735 calculates a normalization factor 1736, for a shortest path between two nodes, based on the weights assigned to the edges comprising the shortest path and the maximal path weight 1731. This can be done in numerous ways using statistical methods known to those of ordinary skill in the relevant art. For example in one implementation, a normalization factor Nfₐ, for the shortest path between a node, say node ‘a’ and the root node, can be calculated by the formula:

$${Nf}_{a} = \frac{{W}_{a} + C}{2} ,$$

where Wₐ represents the sum of the weights of the edges in the shortest path.

Similarity generator 1740 generates similarity data 1741 between two nodes by applying normalization factor 1736 to the weights assigned to the edges comprising the shortest path between the two nodes. Similarity data 1741 may pertain to all possible pairs of nodes in an ontological map 1711. For example in one implementation, the similarity data between two nodes, say node ‘x’ and node ‘y’, represented by

$${Sim}_{x , y}^{p} ,$$

can be calculated by the formula:

$${Sim}_{x , y}^{p} = AVG ( {W}_{x}^{p} , {W}_{y}^{p} ) ;$$

where Wₓ^{p} and W_{y}^{p} represent the sum of the weights of the edges that comprise the shortest path, say path ‘p’, from node ‘x’ and node ‘y’ respectively, via the root node, after the normalization factor has been applied. The normalization factor can be applied by using the formulae: Wₓ^{p}=Nfₓ.Wₚ and W_{y}^{p}=Nf_{y}.Wₚ, where Nfₓ is the normalization factor pertaining to node ‘x’ and Nf_{y} is the normalization factor pertaining to the node ‘y’ and Wₚ is the sum of the weights of the edges in the shortest path ‘p’, before the normalization factor has been applied.

The term ‘AVG’ represents that an average of the weights is calculated. This can be done using a number of mathematical formulas, for example, by adding Wₓ^{p} to W_{y}^{p} and then dividing the sum by 2.

Thus, in the above example, the similarity data

$${Sim}_{x , y}^{p}$$

between the two nodes, node ‘x’ and node ‘y’ is the average of Wₓ^{p} and W_{y}^{p}.

Clustering manager 1760 generates cluster data 1761 pertaining to probe-set identifiers 222 based on correlation of probe-set identifiers 222 with probe-set identifier association data 1098, illustrated in FIG. 18A and described below, and similarity data 1741. For example in one implementation, the clustering manager uses the following formula to derive the association of the similarity data to the probe-set identifiers:

$${Sim}_{a , b} = Max ( {Sim}_{x , y}^{p} \vert x ∈ [ {GO}_{a} ] , y ∈ [ {GO}_{b} ] ) ,$$

where Sim_{a,b} is the similarity data between two probe-set identifiers, say ‘a’ and ‘b’; where probe-set identifier ‘a’ is associated with one or more nodes ‘x’, as illustrated by x∈[GOa] and probe-set identifier ‘b’ is associated with one or more nodes ‘y’ as illustrated by y∈[GOb], in the probe-set identifier association data 1098. In the above formula the symbol Max denotes that the maximum value of the similarity data between the one or more nodes ‘x’ and ‘y’ is taken into consideration.

In one implementation, clustering manager 1760 generates cluster data by grouping those probe-set identifiers having similarity data that fall within a specific threshold, while allowing for one probe-set identifier to occur in one or more than one cluster.

Final map generator 1755 generates final ontological map 1756 based at least in part upon the occurrence of probe-set identifiers 222 in the sets or groups comprising cluster data 1761 and in the modified ontological map 1746. The generation of final ontological map 1756 could include, but is not limited to, coloring or highlighting nodes associated with probe-set identifiers 222 in modified ontological map 1746.

Ontological maps 1711, 1746 and 1756 maybe displayed in a graphical user interface and each node may display a Gene Ontology category or term and the number of probe-set identifiers associated with that node. Selecting a node can initiate a display of a list of the probe-set identifiers associated with that node in the GUI. Cluster data 1761 may include probe-set identifiers 222 divided into various sets or groups such that a probe-set identifier may occur in more than one such sets or groups. These sets or groups may be displayed in a user interface as described above. FIG. 18A is a graphical representation of one embodiment of probe-set identifier association data 1098. In this implementation, probe-set identifier association data 1098 includes a correlation of known probe-set identifiers with Gene Ontology categories or terms. In the illustrated implementation, this data may be represented in a tabular format and may be so displayed in a graphical user interface. In an illustrative example, presence or absence of association of a probe-set identifier 222 with one or more Gene Ontology categories or terms 1810 may be displayed in a table, the rows in the table (except for a title row) contain probe-set identifiers 222 and other columns (except the first column that identifies the probe sets) contain data regarding the association of the one or more probe-set identifiers. In this implementation, the presence of association between a probe-set identifier is indicated by a numeral ‘1’ in the corresponding column (see, e.g., association indicator 1820B) and the absence of association is depicted by the numeral ‘0’ (see, e.g., association indicator 1820A). The resulting table may be thought of as providing to the user a “fingerprint” of ontological categories or terms associated with each of a set of user-selectable probe-set identifiers (i.e., the rows of the table of FIG. 18A). This fingerprint provides a shortcut representation of this association that is useful in both conveying the information visually (and allowing interactive visual comparisons among characteristics of various probe sets). It also provides a computationally efficient format for automatic comparison and processing of association information. For example, a first probe-set identifier may have associated with it a first set of association indicators that can be compared against a second set of association indicators associated with a second probe-set identifier. This comparison of association indicators typically is computationally much faster than directly comparing, for example, Gene Ontology terms associated with the two probe-set identifiers. Therefore, a comparison of association indicators rapidly indicates presence or absence of similarity between the two probe-set identifiers and facilitates a computationally efficient comparison. Moreover, as will now be evident to those of ordinary skill in the relevant art, numerous other formats and textual and/or graphical representations of presence or absence of association between any probe-set identifiers and any Gene Ontology terms or categories are possible. Thus the example shown in FIG. 18A is illustrative only, and not limiting or restrictive in any manner. In the same or other embodiment, data 1098 may include correlation of probe set identifiers to other terms not necessarily restricted to Gene Ontology categories or terms. For example, data 1098 may include a correlation of known probe set identifiers to “experimental terms” which describe experimental conditions pertaining to microarray or other biological experiments. A further example of such “experimental terms” may include the terms and categories in the MGED ontology of the Microarray Gene Expression Data Society (http://www.mged.org/). The MGED ontology provides standard terms for annotation of microarray experiments to enable unambiguous descriptions of how the experiment was performed and may be used by investigators annotating their microarray or other biological experiments. In the MGED ontology the terms are organized into classes with properties. The terms enable structured queries of elements of the experiments. The various categories under which the terms are organized include Classes (e.g. Age #1, Allele #1, ArrayDesignPackageArrayManufacture #7, Atmosphere #1 and so on), Properties (e.g. ID #1, accession #1, accession_version #1, address #1, authors #1, biomaterial_amount #1, biomaterial_purity #1, biosource_type #1, description #1 and so on) and Individuals (e.g. 32P, 33P, A #1, Biotin, CABRI #1, CABRI_linenamesahc #1, CASRegistryNumber #1, CBIL_CV #1, CBIL_anat_id #1, ChemID #1, Cy3, Cy5, DNA and so on). Further examples and details are available on the website: http://mged.sourceforge.net/ontologies/MGEDontology.php.

The ontology is not fixed or unchanging and is expected to continually grow, especially as new applications of microarray and other technology arise that require descriptive terms. Thus, data 1098 may include correlation of probe set identifiers to other terms not necessarily ontological. The preceding examples are only illustrative and not limiting or restrictive in any manner.

FIG. 18B is a graphical representation of one embodiment of database schema 1830 suitable for storing graph traversal and connectivity data 1099 in the local genomic and/or product database 518. As will be appreciated by those of ordinary skill in the relevant art, database schema 1830 is not the only manner in which connectivity data 1099 may be stored in database 518; numerous other variations of schema 1830 may be suitably employed for this purpose. In this implementation, schema 1830 is designed to store biological knowledge that may be collected, for example, from public sources such as Locuslink, Unigene, SwissTrEMBL, and so on, and organized into a relational database following the concept of the central dogma of molecular biology. The database entities are modeled after biological entities and the relationships between them. For instance, one genetic locus can produce one or more transcripts, one transcript can generate zero to many proteins, and one protein may have zero or many domains. Various tables containing information regarding locus, transcript, protein, and domain may be designed to implement these relationships. Thus, table locus is linked to table transcript with a one-to-many relationship, table transcript is linked to table protein with a zero-to-many relationship, and table protein is linked to table domain with a zero-to-many relationship. Two tables in the illustrated implementation are designed to represent the digraph structure of GO, one table for GO terms (nodes) and one table for the parent-child relationship between two terms (edge). Since one gene may have many GO term annotations and one GO term may be used in the annotations for many genes, the GO annotations for specific genes are stored in the join table in between locus table and GO-terms table. Tables holding Affymetrix probe sets (described and available at www.netaffx.com) are associated with the locus table in this implementation through Genbank accession tables. Through the locus table, the connection between Affymetrix probe sets and GO annotations are established.

In order to eliminate the need for real-time path enumeration, a graph algorithm may be used to resolve all possible full or partial paths for every given GO node. The results from path enumeration are stored into several derived tables in the database. Since the terms within each of the three GO categories are unique in this implementation to the categories, each GO category is treated as an independent digraph and separate tables are created to hold the path enumeration results. Thus, the root for each sub-digraph is “molecular function,” “biological process,” or “cellular component.” Querying derived path tables allows rapid calculation of similarity between two nodes based on the maximum length of shared partial paths between two given nodes.

As is evident from the preceding descriptions, probe-set identifiers and their underlying probe sets are important references for analysis with respect to numerous features of genomic web portal 400. It often is therefore of particular interest to a user to have an efficient and informative visual representation of probe sets of interest. For example, a user may wish to know how the probes of a probe set of interest were designed and how the probes align with other probe sets, with genes or ESTs of interest, or with other types of sequences. Among the reasons that a user may wish to have this information are to confirm that the probe sequences are unique and thus not capable of detecting genes unrelated to the gene the probe was designed to detect, to confirm that the targets used with the probes are capable of efficiently hybridizing with the probes, to understand why gene fragments in a sample that do not align with probes were not detected, and for other reasons. In this context the words “align” and “alignment” are to be interpreted broadly to include various techniques and measures of sequence similarity as are well known to those of ordinary skill in the art. Similarly, “misalignment” is intended to refer broadly to an absolute or relative lack of alignment as indicated or measured in various conventional ways.

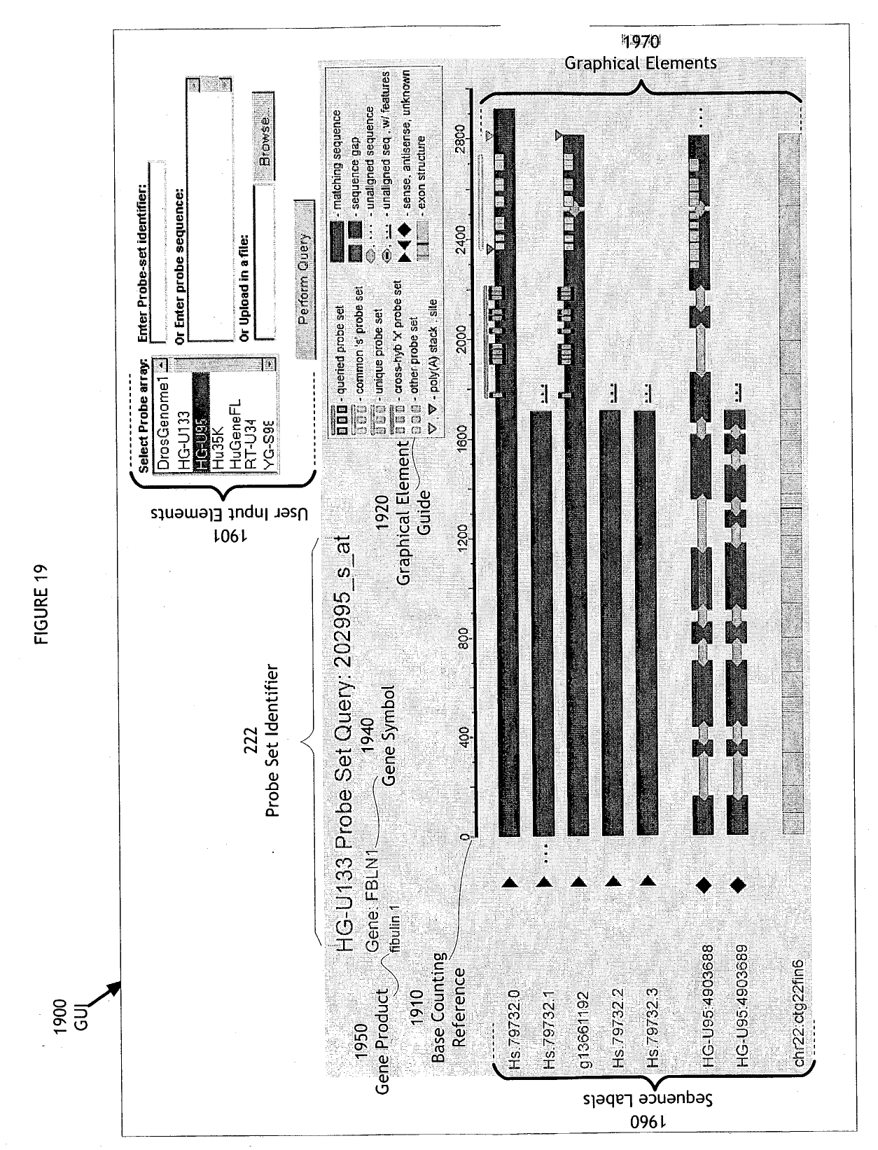

FIG. 19 is a graphical representation of one embodiment of a graphical user interface that addresses these, and other, needs and interests. GUI 1900 of FIG. 19 is suitable for providing information pertaining to one or more probe sequences related to one or more probes of one or more probe sets (hereafter referred to for convenience simply as probe sets in this context), including probe sets represented by one or more probe-set identifiers 222. In addition, GUI 1900 may display information pertaining to one or more probes or probes set sequences of a custom probe set (e.g., probe sets requested by a user), as well as alignment, content, and other information related to genomic sequences, alternative splice variants, ESTs, RNAs, mRNAs, gene regions, genes or chromosomes.

In an illustrative example, GUI 1900 includes one or more user input elements 1901, a base-counting reference 1910 (a plurality of base-counting references could be included in other implementations), one or more graphical element guides 1920, a (or a plurality of in other examples) particular (e.g., user-selected) probe-set identifier 222 pertaining to the one or more probe set sequences being displayed, one or more gene symbols 1940, one or more gene product labels 1950, one or more sequence labels 1960, and one or more graphical elements 1970 each corresponding to at least one of the plurality of sequence labels 1960.

User input elements 1901 include various elements suitable for receiving, in accordance with any of a variety of conventional techniques, one or a combination of probe-set identifiers 222 and/or probe sequences. Furthermore, in some implementations, elements 1901 include an element for selecting one or more probe arrays that the user wishes to specify with respect to querying database 518 for an alignment to be displayed in the same or different instance of GUI 1900 or a variation or other implementation thereof. The alignment may be displayed graphically in GUI 1900, as described below. In addition, the input elements may include any conventional graphical element or technique for receiving from user 101 an indication of the amount or level of misalignment that is allowable when performing a query. The term “probe sequence” includes in its meaning in this context the sequence of bases, residues or other elementary components of the probe. For example, probes made up of DNA or RNA would have probe sequences made up of the corresponding bases, and probes made up of peptides would have probe sequences of amino acids. In some contexts, the term “probe sequence” refers to a graphical representation (e.g., a bar or line) having a length or other attribute, such as for example color, that indicates the extent, alignment, or other correlation of a probe's sequence with another sequence, such as a biological or genomic sequence or a reference sequence (e.g., a bar or line representing a reference series of nucleic acid or amino acid residues).

Base-counting reference 1910 displays on a numerical scale a range of bases (or other residues in alternative implementations) typically including within the range the bases of the probe set sequence on display in the graphical user interface. The number of bases of the probe set sequence on display may be changed by user 101, and such a change may correspondingly alter the numerical scale of the base counting reference 1910. Initial or other reference points determining the scale of reference 1910 may be user selectable so that, for example, bases are counted from the beginning of a gene of interest to user 101 (or a particular regulatory or other site related to the gene), the beginning or other reference point on a chromosome that includes the gene of interest, and so on.

Graphical element guide 1920 displays information related to the one or more graphical elements 1970 described below. Graphical element guide 1920 is a guide to the information displayed by graphical elements 1970, including but not limited to:

(a) probe set sequences of the one or more probe sets, including probe sets represented by one or more probe-set identifier 222;

(b) probe set sequences that are biologically or otherwise related to the probe set sequences being displayed (e.g., as identified as being related in accordance with one or more of the graphical user interfaces described above in relation to FIGS. 16A and 16B and implementing components of web portal 400);

(c) one or more genomic sequences, ESTs, RNAs, gene regions, genes, or chromosomes that are biologically or otherwise related to the probe set sequences being displayed;

(d) gaps in the one or more genomic sequences, ESTs, RNAs, gene regions, genes, or chromosomes that are biologically or otherwise related to the probe set sequences being displayed;

(e) biologically or otherwise unaligned regions in the one or more genomic sequences, ESTs, RNAs, gene regions, genes, or chromosomes that are biologically or otherwise related to the probe set sequences being displayed;

(f) biological or other orientation of the one or more genomic sequences, ESTs, RNAs, gene regions, genes, or chromosomes that are biologically or otherwise related to the probe set sequences being displayed (e.g., sense, anti-sense or unknown orientation);

(g) one or more genomic sequences, ESTs, RNAs, gene regions, genes, or chromosomes from the genome of one or more life forms including but not limited to the genome of one or more animals, plants, fungi, prokaryotes, eukaryotes, viruses and prions.

Gene Symbol 1940 (FBLN1 in this example) represents the name or other identifiers associated with at least one genomic sequence, EST, RNA, gene region, gene or chromosome to which the probe set sequence being displayed is compared or aligned, wherein the probe set sequence being displayed includes probe set sequences of the one or more probe sets, including probe sets represented by one or more probe-set identifiers 222. This comparison or alignment may carried out by numerous methods well known to those of ordinary skill in the art, including but not limited to, for example BLAST, PSI-BLAST, MEGABLAST, RPS-BLAST, and so on. Gene product label 1950 (fibulin 1 in this example) represents the name or other identifiers associated with the biological product of the genomic sequence, EST, RNA, gene region, gene or chromosome represented by the Gene Symbol 1940.

Sequence labels 1960 represent the name or other identifiers associated with graphical elements 1970, including but not limited to exemplar sequence labels; UniGene cluster identifiers; or UniGene cluster identifiers with additional information, for example sub-clusters derived from re-clustering the UniGene cluster labeled by adding a digit after a period on the original UniGene cluster ID. As will be appreciated by those of ordinary skill in the relevant art, numerous other identifiers associated with graphical elements 1970 may be displayed. In addition, user 101 may interact with sequence labels 1960, for example by clicking on them, to display further information about the probe set sequences, genomic sequences, ESTs, RNAs, gene regions, genes, or chromosomes that are associated with sequence labels 1960 and consequently with graphical elements 1970. This further information may be displayed in the same graphical user interface or by launching another user interface.

Elements 1970 graphically represent the above mentioned comparison or alignment of one or more probe set sequences represented by the one or more probe-set identifiers 222 against other one or more probe set sequences represented by the one or more probe-set identifiers 222, genomic sequences, ESTs, RNAs, gene regions, genes, chromosomes, or other related sequences (e.g., corresponding amino acid sequences).

In the illustrated embodiment, the uppermost horizontal bar represents the probe sequences of probe sets represented by one or more probe-set identifiers 222, hereafter referred to as the “query sequence” that was used in the comparison or alignment, e.g., by a BLAST alignment. The bars below represent the one or more probe set sequences, genomic sequences, ESTs, RNAs, gene regions, genes, or chromosomes, hereafter referred to as “subject sequences” against which the query sequence was compared or aligned.

Additionally, the following criteria may be applied to simplify the comparison or alignment display:

i. Sequences may be included in the comparison or alignment display only when a user-specified or predetermined percentage (e.g., 40%) or more of the query sequence aligns to the subject sequence or vice versa, e.g., 40% or more of the subject sequence aligns to the query sequence.

ii. Self-alignments are used to subtract a numerical value “delta” from the query sequence size and the subject sequence size before calculating the percent of sequences aligned. This delta may be based on the number of biological or biochemical units comprising the query sequence and the subject sequence, for example nucleotide bases, that do not align to a sequence itself within the region bounded by the query sequence or subject sequence alignment.

iii. The fraction of the query sequence or subject sequences aligned may be based on the length of the shortest from amongst the query sequence or subject sequences, less the above mentioned delta value.

In some embodiments, user 101 may interact with graphical elements 1970 to view additional information, including but not limited to the information displayed in graphical element guide 1920. This interaction may be accomplished, for example, by moving the pointer of a graphical user interface pointing device, for example a mouse device, over the graphical elements 1970, by clicking on a part of the graphical elements 1970, or by numerous other conventional methods for interacting with the graphical user interface. The additional information may be displayed in the same graphical user interface or by launching another user interface.

Additional interactive features of GUI 1900 may include selecting at least one of a graphical element 1970 by moving a cursor via mouse or keyboard and clicking the button on the mouse, or pressing the enter key on the keyboard, or other method commonly used for selecting elements. When a user selects an element or elements, portal 400 may alter the display in the graphical user interface and/or present one or more additional graphical user interfaces, or windows.

As will now be appreciated by those of ordinary skill in the relevant art in light of this disclosure, the above described graphical user interface may be used as a tool to display a very wide range of information, including biological information, that lends itself to linear comparison and visualization. Furthermore, the above mentioned description is illustrative only and does not limit the invention any way whatsoever.

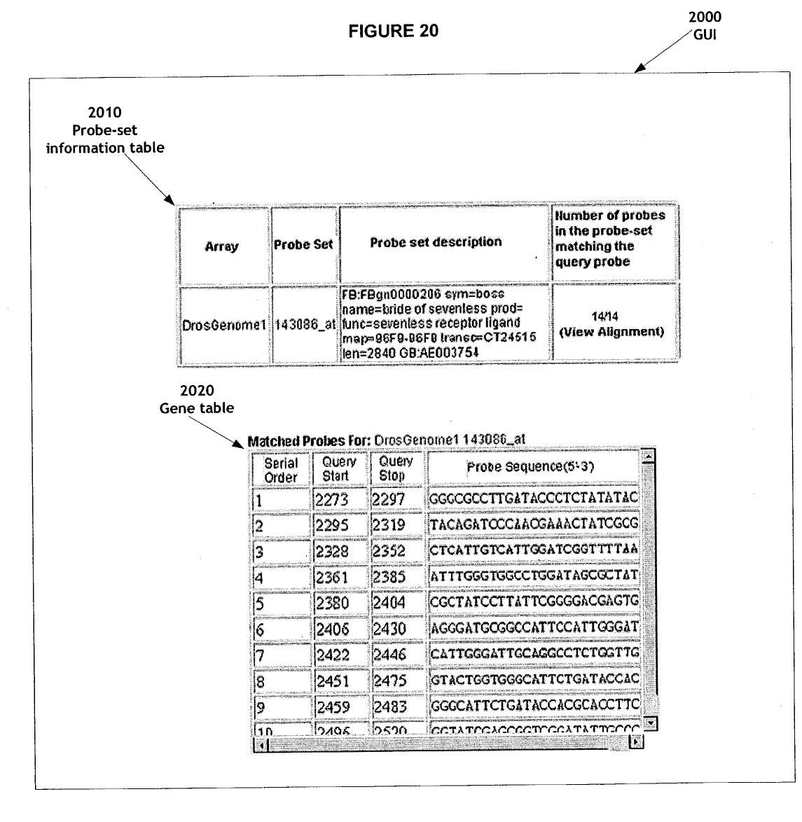

In accordance with some implementations, user 101 may be provided with information in a tabular format pertaining to one or more probe sequences of a probe set related to probe-set identifiers. FIG. 20 is a graphical representation of a graphical user interface suitable for providing this information in such a format. In an illustrative example, GUI 2000 includes one or more probe-set information tables 2010 and/or gene tables 2020. In this example, table 2010 displays information regarding one or more probe arrays under the heading “Array” (e.g., “DrosGenome1” as shown), one or more corresponding probe-set identifiers under the heading “Probe Set” (e.g. “143086_at” as shown), detailed description of the relevant probe sets under the heading “Probe set Description,” and additional information including but not limited to the number of probes in the probe-set matching the query probe (e.g., 14/14). All the elements comprising table 2010 may be interactive and capable of receiving user input. In an illustrative example, clicking on a probe-set identifier under the heading “Probe Set” displays additional information regarding the probe set associated with the probe-set identifier in one or more gene tables 2020. Alternatively, user 101 may select an element of table 2010 to provide further information; for example, the user may click on the number of probes in the probe-set matching the query probes (e.g. 14/14) to display the graphical alignment of the query sequence and one or more probe sequences comprising the probe set. This information may be presented in a graphical user interface similar to GUI 1900.

Gene table 2020 is an illustrative example of techniques for displaying data pertaining to one or more probe sets associated with one or more probe-set identifier 222. In this illustrative example of what may hereafter be referred to as a “gene table,” table 2020 displays the information under various columns including, but not limited to, columns named “Serial Order” that displays a numerical serial of the probe sequences; “Query Start” that displays the number of a sequence element, e.g., a base in case of a DNA molecule, at which the alignment of the query sequence begins; “Query stop” that displays the number of a sequence element, e.g., a base in case of a DNA molecule, at which the alignment of the query sequence ends; and “Probe Sequence (5′-3′)” that displays the probe sequence in a 5′ to 3′ direction (in this example). Furthermore, all the elements of table 2020 may be interactive and capable of receiving user input. For example, user 101 may click on an element of table 2020 to display the graphical alignment of the query sequence, and one or more probe sequences comprising the probe set, in a graphical user interface similar to GUI 1900.

As used herein, the term “graphical user interface” is intended to be broadly interpreted so as to include various ways of communicating information to, and obtaining information from, a user. For example, information may be sent to a user in an email as an alternative to, or in addition to, presenting the information on a computer screen employing graphical elements (such as shown illustratively in FIGS. 16A and 16B). As is known by those of ordinary skill in the relevant art, the email may include graphics, or be designed to invoke graphics; similar to those that may be displayed in an interactive graphical user interface.

As indicated above, functional elements of portal 400 may be implemented in hardware, software, firmware, or any combination thereof. In the embodiment described above, it generally has been assumed for convenience that the functions of portal 400 are implemented in software. That is, the functional elements of the illustrated embodiment comprise sets of software instructions that cause the described functions to be performed. These software instructions may be programmed in any programming language, such as Java, Perl, C++, another high-level programming language, low-level languages, and any combination thereof. The functional elements of portal 400 may therefore be referred to as carrying out “a set of genomic web portal instructions,” and its functional elements may similarly be described as sets of genomic web portal instructions for execution by servers 510, 520, and 530.

In some embodiments, a computer program product is described comprising a computer usable medium having control logic (computer software program, including program code) stored therein. The control logic, when executed by a processor, causes the processor to perform functions of portal 400 as described herein. In other embodiments, some such functions are implemented primarily in hardware using, for example, a hardware state machine. Implementation of the hardware state machine so as to perform the functions described herein will be apparent to those skilled in the relevant arts.

Aspects of probe selection and design and other features applicable to implementations of the present invention are described in greater detail in U.S. patent application Ser. Nos. 10/028,884, 10/027,682, 10/028,416, and 10/006,174, all of which are hereby incorporated by reference herein in their entireties for all purposes.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways and by various elements in alternative embodiments. For example, some or all of the functions described as being carried out by determiner 820 could be carried out by correlator 830, or these functions could otherwise be distributed among other functional elements. Also, the functions of several elements may, in alternative embodiments, be carried out by fewer, or a single, element. For example, the functions of determiner 820 and correlator 830 could be carried out by a single element in other implementations. Similarly, in some embodiments, any functional element may perform fewer, or different, operations than those described with respect to the illustrated embodiment. Also, functional elements shown as distinct for purposes of illustration may be incorporated within other functional elements in a particular implementation. For example, the division of functions between an application server and a network server of the genome portal is illustrative only. The functions performed by the two servers could be performed by a single server or other computing platform, distributed over more than two computer platforms, or other otherwise distributed in accordance with various known computing techniques.

Also, the sequencing of functions or portions of functions generally may be altered. Certain functional elements, files, data structures, and so on, may be described in the illustrated embodiments as located in system memory of a particular computer. In other embodiments, however, they may be located on, or distributed across, computer systems or other platforms that are co-located and/or remote from each other. For example, any one or more of data files or data structures described as co-located on and “local” to a server or other computer may be located in a computer system or systems remote from the server. In addition, it will be understood by those skilled in the relevant art that control and data flows between and among functional elements and various data structures may vary in many ways from the control and data flows described above or in documents incorporated by reference herein. More particularly, intermediary functional elements may direct control or data flows, and the functions of various elements may be combined, divided, or otherwise rearranged to allow parallel or distributed processing or for other reasons. Also, intermediate data structures or files may be used and various described data structures or files may be combined or otherwise arranged. Numerous other embodiments, and modifications thereof, are contemplated as falling within the scope of the present invention as defined by appended claims and equivalents thereto.

What is claimed is:

1. A system for providing correlations between one or more probe-set identifiers and one or more biological sequences, comprising:
an input manager constructed and arranged to receive a user selection of the probe-set identifiers;
a correlator constructed and arranged to associate the probe-set identifiers with probe sequences and to correlate the probe sequences with the one or more biological sequences; and
an output manager constructed and arranged to enable for display a graphical user interface including one or more graphical elements representing one or more of the probe sequences associated with the probe-set identifiers and one or more graphical elements representing the one or more biological sequences.

2. The system of claim 1, wherein:
the one or more biological sequences include any one or more of sequences of EST, DNA, cDNA, RNA, cRNA, mRNA, gene region, gene, gene transcripts, gene transcription products, chromosome, peptide, protein fragment, protein, or residue counting reference.

3. The system of claim 1, wherein:
the probe-set identifiers or probe sequences are associated with probes of a probe array constructed and arranged to detect or measure any one or any combination of biological, biochemical or genetic metrics including gene expression, genotype, SNP, haplotype, or targets including antibodies, cell membrane receptors, monoclonal antibodies and antisera reactive with specific antigenic determinants, drugs, oligonucleotides, nucleic acids, peptides, proteins, cofactors, lectins, sugars, polysaccharides, cells, cellular membranes, or organelles.

4. A method for providing correlations between one or more probe-set identifiers and one or more biological sequences, comprising the acts of:
associating the probe-set identifiers with probe sequences; and
correlating the probe sequences with the one or more biological sequences.

5. The method of claim 4, wherein:
each probe-set identifier identifies only one probe set comprised of a unique set of one or more probe sequences.

6. The method of claim 4, further comprising the act of:
receiving a user selection of the probe-set identifiers.

7. The method of claim 6, wherein:
the user selection is provided, at least in part, using the Internet.

8. The method of claim 6, wherein:
the user selection is provided, at least in part, using a graphical user interface.

9. The method of claim 8, wherein:
the graphical user interface includes elements for receiving a user selection including a list of probe-set identifiers in a computer-readable file.

10. The method of claim 8, wherein:
the graphical user interface includes one or more user input elements for receiving a user selection of one or more query biological sequences, or identifiers of one or more query biological sequences; and
the method further includes the act of associating the one or more query biological sequences, or their identifiers, with one or more probe-set identifiers.

11. The method of claim 10, wherein:
the query biological sequences include any one or more of sequences of EST, DNA, cDNA, RNA, cRNA, mRNA, gene region, gene, gene transcripts, gene transcription products, chromosome, peptide, protein fragment, or protein.

12. The method of claim 10, wherein:
the elements for receiving a user selection further include a probe-array type interface that accepts a user selection of one or more probe-array types to indicate that the user selection of the probe-set identifiers is to be limited to probe-set identifiers associated with the selected probe-array types.

13. The method of claim 10, wherein:
the act of associating the query biological sequences, or their identifiers, with the probe-set identifiers includes using a predetermined or a user-selected margin of allowable misalignment between the biological sequence and the probe sequences associated with the probe-set identifiers.

14. The method of claim 4, further comprising the act of:
receiving a selection of the probe-set identifiers that is based, at least in part, on results of at least one experiment with one or more probe arrays having probe sets identified by the probe-set identifiers.

15. The method of claim 4, wherein:
the act of correlating includes the act of aligning the probe sequences with the one or more biological sequences.

16. The method of claim 15, wherein:
the act of aligning includes using a predetermined or a user-selected margin of allowable misalignment.

17. The method of claim 4, further comprising the act of:
enabling the correlation for display.

18. The method of claim 17, wherein:
the act of enabling the correlation for display includes providing a graphical user interface including one or more graphical elements representing one or more of the probe sequences associated with the probe-set identifiers and one or more graphical elements representing the one or more biological sequences.

19. The method of claim 18, wherein:
the one or more biological sequences include any one or more of sequences of EST, DNA, cDNA, RNA, cRNA, mRNA, gene region, gene, gene transcripts, gene transcription products, chromosome, peptide, protein fragment, protein, or residue counting reference.

20. The method of claim 18, wherein:
the one or more graphical elements representing the one or more probe sequences are graphically aligned with the one or more graphical elements representing the one or more biological sequences.

21. The method of claim 20, wherein:
the one or more biological sequences include any one or more of sequences of EST, DNA, cDNA, RNA, cRNA, mRNA, gene region, gene, gene transcripts, gene transcription products, chromosome, peptide, protein fragment, protein, or residue counting reference.

22. The method of claim 18, wherein:
the one or more graphical elements representing the one or more probe sequences are associated by color with the one or more graphical elements representing one or more biological sequences.

23. The method of claim 18, wherein:
at least one of the one or more biological sequences is associated with one or more sequence labels.

24. The method of claim 23, wherein:
the one or more sequence labels include one or more of an accession number, a gene identifier, or a genomic sequence identifier.

25. The method of claim 17, wherein:
the act of enabling the correlation for display includes providing a graphical user interface having one or more tabular representations of the correlation.

26. The method of claim 25, wherein:
the one or more tabular representations include one or more gene tables.

27. The method of claim 26, wherein:
the one or more gene tables include one or more biological sequences.

28. The method of claim 27, wherein:
the biological sequences include sequences correlated with one or more query sequences.

29. The method of claim 25, wherein:
the one or more tabular representations include one or more probe-set information tables.

30. The method of claim 29, wherein:
the one or more probe-set information tables include a description of one or more probe sets.

31. An apparatus for providing correlations between one or more probe-set identifiers and one or more biological sequences, comprising:
an associator constructed and arranged to associate the probe-set identifiers with probe sequences; and
a correlator constructed and arranged to correlate the probe sequences with the one or more biological sequences.

32. The apparatus of claim 31, wherein:
each probe-set identifier identifies only one probe set comprised of a unique set of one or more probe sequences.

33. The apparatus of claim 31, wherein:
the probe-set identifiers are selected by a user based, at least in part, on results of at least one experiment with one or more probe arrays having probe sets identified by the probe-set identifiers.

34. A system for providing correlations between one or more probe-set identifiers and one or more biological sequences, comprising:
an input manager constructed and arranged to receive a user selection of the probe-set identifiers; and
a correlator constructed and arranged to associate the probe-set identifiers with probe sequences and to correlate the probe sequences with the one or more biological sequences.

35. The system of claim 34, wherein:
each probe-set identifier identifies only one probe set comprised of a unique set of one or more probe sequences.

36. The system of claim 34, wherein:
the user selection is provided, at least in part, using a graphical user interface that includes one or more user input elements for receiving a user selection of one or more query biological sequences, or identifiers of one or more query biological sequences; and
the correlator further is constructed and arranged to associate the one or more query biological sequences, or their identifiers, with one or more probe-set identifiers.

37. The system of claim 36, wherein:
the query biological sequences include any one or more of sequences of EST, DNA, cDNA, RNA, cRNA, mRNA, gene region, gene, gene transcripts, gene transcription products, chromosome, peptide, protein fragment, or protein.

38. The system of claim 36, wherein:
the elements for receiving a user selection further include a probe-array type interface that accepts a user selection of one or more probe-array types to indicate that the user selection of the probe-set identifiers is to be limited to probe-set identifiers associated with the selected probe-array types.

39. The system of claim 36, wherein:
the correlator further is constructed and arranged to associate the query biological sequences, or their identifiers, with the probe-set identifiers using a predetermined or a user-selected margin of allowable misalignment between the biological sequence and the probe sequences associated with the probe-set identifiers.

40. The system of claim 34, further comprising:
an input manager constructed and arranged to receive a selection of the probe-set identifiers that is based, at least in part, on results of at least one experiment with one or more probe arrays having probe sets identified by the probe-set identifiers; and
an output manager constructed and arranged to enable display of the correlation in a graphical user interface including one or more graphical elements representing one or more of the probe sequences associated with the probe-set identifiers and one or more graphical elements representing the one or more biological sequences.

41. The system of claim 40, wherein:
the one or more biological sequences include any one or more of sequences of EST, DNA, cDNA, RNA, cRNA, mRNA, gene region, gene, gene transcripts, gene transcription products, chromosome, peptide, protein fragment, protein, or residue counting reference.

42. The system of claim 40, wherein:
the one or more graphical elements representing the one or more probe sequences are graphically aligned with the one or more graphical elements representing one or more biological sequences.

43. The system of claim 42, wherein:
the one or more biological sequences include any one or more of sequences of EST, DNA, cDNA, RNA, cRNA, mRNA, gene region, gene, gene transcripts, gene transcription products, chromosome, peptide, protein fragment, protein, or residue counting reference.

44. The system of claim 40, wherein:
the one or more graphical elements representing the one or more probe sequences are associated by color with the one or more graphical elements representing one or more biological sequences.

45. The system of claim 40, wherein:
at least one of the one or more biological sequences is associated with one or more of an accession number, a gene identifier, or a genomic sequence identifier.

46. The system of claim 40, wherein:
the graphical user interface further includes one or more tabular representations of the correlation.

47. The system of claim 46, wherein:
the one or-more tabular representations include one or more gene tables having one or more biological sequences including sequences correlated with one or more query sequences.

48. The system of claim 46, wherein:
the one or more tabular representations include one or more probe-set information tables including a description of one or more probe sets.

49. A system for providing correlations between one or more probe-set identifiers and one or more biological sequences, comprising:
means for receiving a user selection of the probe-set identifiers using a first graphical user interface;
means for associating the probe-set identifiers with probe sequences;
means for correlating the probe sequences with the one or more biological sequences; and
means for enabling the correlation for display including providing a second graphical user interface including one or more graphical elements representing one or more of the probe sequences associated with the probe-set identifiers and one or more graphical elements representing the one or more biological sequences.

50. The system of claim 49, wherein:
the first and second graphical user interfaces are included in a same graphical user interface.

51. The system of claim 49, wherein:
each probe-set identifier identifies only one probe set comprised of a unique set of one or more probe sequences.

52. A computer-implemented graphical user interface for providing correlations between one or more probe-set identifiers and one or more biological sequences, comprising:
one or more first graphical elements representing one or more probe sequences associated with the probe-set identifiers; and
one or more second graphical elements representing the one or more biological sequences;
wherein the first and second graphical elements are graphically aligned with each other.

53. The graphical user interface of claim 52, wherein:
the one or more biological sequences include any one or more of sequences of EST, DNA, cDNA, RNA, cRNA, mRNA, gene region, gene, gene transcripts, gene transcription products, chromosome, peptide, protein fragment, protein, or residue counting reference.

54. The graphical user interface of claim 52, wherein:
the first graphical elements are associated by color with the second graphical elements.

55. The graphical user interface of claim 52, wherein:
each probe-set identifier identifies only one probe set comprised of a unique set of one or more probe sequences.

56. The graphical user interface of claim 52, further comprising:
one or more user input elements for receiving a user selection of one or more query biological sequences, or identifiers of one or more query biological sequences.

57. The graphical user interface of claim 56, wherein:
the query biological sequences include any one or more of sequences of EST, DNA, cDNA, RNA, cRNA, mRNA, gene region, gene, gene transcripts, gene transcription products, chromosome, peptide, protein fragment, or protein.

58. The graphical user interface of claim 56, wherein:
the elements for receiving a user selection further include a probe-array type interface that accepts a user selection of one or more probe-array types to indicate that a user selection of probe-set identifiers is to be limited to probe-set identifiers associated with the selected probe-array types.

59. A graphical user interface generated by a method for providing correlations between one or more probe-set identifiers and one or more biological sequences, comprising the acts of:
associating the probe-set identifiers with probe sequences; and
correlating the probe sequences with the one or more biological sequences;
wherein the graphical user interface includes one or more first graphical elements representing the one or more probe sequences graphically aligned with one or more second graphical elements representing the one or more biological sequences.

60. The graphical user interface of claim 59, wherein:
each probe-set identifier identifies only one probe set comprised of a unique set of one or more probe sequences.
